(19) United States
(12) Patent Application Publication
(10) Pub. No.: US 2022/0415443 A1
(43) Pub. Date: Dec. 29, 2022

(54) MACHINE-LEARNING MODEL FOR GENERATING CONFIDENCE CLASSIFICATIONS FOR GENOMIC COORDINATES

(71) Applicants: Illumina, Inc., San Diego, CA (US); Illumina Cambridge Limited, Cambridge (GB)

(72) Inventors: Mitchell A. Bekritsky, Decatur, GA (US); Camilla Colombo, Cambridge (GB); Dorna KashefHaghighi, Menlo Park, CA (US); Rohan Paul, Palo Alto, CA (US); Fabio Zanarello, Newquay (GB); Tevfik Umut Dincer, Los Angeles, CA (US); Nathan Harwood Johnson, Drexel Hill, PA (US)

(21) Appl. No.: 17/808,902

(22) Filed: Jun. 24, 2022

Related U.S. Application Data
(60) Provisional application No. 63/216,382, filed on Jun. 29, 2021.

(51) Int. Cl. G16B 30/10; G16B 40/20

(52) U.S. Cl. CPC G16B 30/10; G16B 40/20

(57) ABSTRACT

This disclosure describes methods, non-transitory computer readable media, and systems that can train a genome-location-classification model to classify or score genomic coordinates or regions by the degree to which nucleobases can be accurately identified at such genomic coordinates or regions. For instance, the disclosed systems can determine sequencing metrics for sample nucleic-acid sequences or contextual nucleic-acid subsequences surrounding particular nucleobase calls. By leveraging ground-truth classifications for genomic coordinates, the disclosed systems can train a genome-location-classification model to relate data from one or both of the sequencing metrics and contextual nucleic-acid subsequences to confidence classifications for such genomic coordinates or regions. After training, the disclosed systems can also apply the genome-location-classification model to sequencing metrics or contextual nucleic-acid subsequences to determine individual confidence classifications for individual genomic coordinates or regions and then generate at least one digital file comprising such confidence classifications for display on a computing device.

### CROSS-REFERENCE TO RELATED APPLICATIONS

This application claims the benefit of, and priority to, U.S. Provisional Application No. 63/216,382, entitled “MACHINE-LEARNING MODEL FOR GENERATING CONFIDENCE CLASSIFICATIONS FOR GENOMIC COORDINATES,” filed Jun. 29, 2021, the contents of which are hereby incorporated by reference in their entirety.

### BACKGROUND

In recent years, biotechnology firms and research institutions have improved hardware and software for sequencing nucleotides and identifying variant calls for samples containing nucleobases that differ from a norm or a reference genome. For instance, some existing nucleic-acid-sequencing platforms determine individual nucleobases of nucleic-acid sequences by using conventional Sanger sequencing or by using sequencing-by-synthesis (SBS). When using SBS, existing platforms can monitor thousands, tens of thousands, or more nucleic-acid polymers being synthesized in parallel to detect more accurate nucleobase calls from a larger base-call dataset. For instance, a camera in SBS platforms can capture images of irradiated fluorescent tags from nucleobases incorporated into to such oligonucleotides. After capturing such images, existing SBS platforms send base-call data (or image data) to a computing device with sequencing-data-analysis software to determine a nucleobase sequence for a nucleic-acid polymer (e.g., exon regions of a nucleic-acid polymer) and use a variant caller to identify any single nucleotide variants (SNVs), insertions or deletions (indels), or other variants within a sample's nucleic-acid sequence.

Despite these recent advances in sequencing and variant calling, existing sequencing-data-analysis software often includes a variant caller that identifies nucleotide variants regardless (or without indication) of the position of the nucleotide variant within a sequence or genome. Because the context of a variant call's position can influence the reliability of the call—with certain genomic regions more likely to exhibit predictable sequences and other genomic regions more likely to exhibit variation—the location of a nucleotide variant can affect the probability of identifying a variant as a true positive or a false positive. Further to the point, the probability of correctly identifying a variant for a given genomic region can differ depending on a specific sequencing method or device. Without a built-in mechanism for analyzing the accuracy of genomic regions and correlating variant calls with such regions—particularly for specific sequencing pipelines—clinicians often use other sequencing methods (e.g., Sanger to supplement SBS sequencing) or supplementary validation tests to orthogonally validate variant calls.

A variant call for a particular variant can range between being inconsequential or critical depending on the genomic region of the variant call. Because existing variant callers often cannot correlate a variant call with accuracy probabilities for a genomic region or position, however, clinicians have limited confidence in the accuracy of variant calls. For example, a variant call identifying a particular single nucleotide polymorphism (SNP) in the hemoglobin beta (HBB) gene can have signification implications. When a variant caller identifies an SNP at rs344 on chromosome 11, the variant caller can either correctly identify the genetic cause of sickle cell anemia or miss the cause of the disease. As a further example, a variant call that correctly or incorrectly identifies the deletion of one or more copies of hemoglobin subunit alpha 1 (HbA1) or hemoglobin subunit alpha 2 (HbA2) genes can result in either correctly identifying a genetic cause of an inherited blood disorder or miss the gene deletion entirely. Accordingly, a variant call for such an SNP or other variant on a gene may be critical but often lack an empirically based indication of accuracy probabilities for the region from which conventional variant callers identify the variant.

Despite the variation in genomic regions for nucleobase calls and the potential importance of variant calls, existing nucleic-acid-sequencing platforms and sequencing-data-analysis software (together and hereinafter, existing sequencing systems) lack an empirically proven way of identifying reportable ranges for regions of higher or lower accuracy within genomes. Such existing sequencing systems likewise lack an empirically proven way of distinguishing between different variant types in such reportable ranges. Existing sequencing systems further lack such empirically proven ways of identifying reportable ranges or distinguishing between variant types within those ranges for specific sequencing pipelines.

Conventionally, clinicians and biotechnology institutions can rely on the characteristics of reference genomes untethered to specific sequencing pipelines. Researchers have identified reportable ranges of regions in reference genomes of higher or lower accuracy, including the high-confidence regions of a reference genome identified by the Genome in a Bottle Consortium (GIAB) and Global Alliance for Genomic Health (GA4GH). But these existing reportable ranges from GIAB and GA4GH limit reportable ranges to benchmark genomic regions at the exclusion of difficult genomic regions, where approximately 79-84% of the human genome is within the benchmark genomic regions; fail to distinguish between different types of accuracy tiers for regions; and do not distinguish reportable ranges by variant type (e.g., SNVs versus indels). With only about 79-84% of a reference genome mapped to benchmark regions and no differentiation in reportable ranges by variant-call type, conventional reportable ranges leave a significant portion of a reference genome without indication of detection accuracy and without indication of whether a specific variant-call type affects detection accuracy.

Even with these conventional reportable ranges, clinicians need specialized knowledge of how characteristics of reference genomes translate to a specific sequencing pipeline to, for example, account for changes to nucleotide sample preparation (e.g., PCR or longer reads), different sequencing devices, or different sequencing-data-analysis software. Indeed, despite reportable ranges of reference genomes, existing sequencing systems cannot identify reportable ranges specific to a sequencing pipeline or derived from empirical data.

In addition to the conventional reportable ranges from GIAB and GA4GH, Illumina, Inc. partnered with research institutions to develop a catalog of high-confidence variant calls in a set of benchmark genomes. By generating whole-genome sequence data for people with a three-generation pedigree and calling variants in each genome, the team developed Platinum Genomes with a catalogue of 4.7 million SNVs and 0.7 million small indels (1-50 base pairs) consistent with the inheritance pattern among these people. While the truthsets of variant calls in Platinum Genomes can be used to verify and measure the performance of variant calls in curated samples, Platinum Genomes and other truthsets from GIAB exclude problematic genomic regions containing both stochastic and systemic errors. Nor can Platinum Genomes or other truthsets account for sample-specific errors in variant calls. Because problematic regions are excluded regardless of the underlying cause for the problem and such a time-intensive cataloguing is difficult (if not impossible) to scale, a catalogue of high-confidence variant calls proves an impractical approach to determining an accuracy and a reliability of variant calls at each genomic coordinate.

### SUMMARY

This disclosure describes embodiments of methods, non-transitory computer readable media, and systems that can train a genome-location-classification model to classify or score genomic coordinates or genomic regions by the degree to which nucleobases can be accurately identified at such genomic coordinates or regions. For example, the disclosed systems can determine one or both of sequencing metrics for diverse sample nucleic-acid sequences and contextual nucleic-acid subsequences surrounding particular nucleobase calls. By leveraging ground-truth classifications for genomic coordinates, in some cases, the disclosed systems train a genome-location-classification model to relate data from one or both of the sequencing metrics and contextual nucleic-acid subsequences to confidence classifications for such genomic coordinates or regions. Having trained such a model, the disclosed systems can likewise apply the genome-location-classification model to data from sequencing metrics or contextual nucleic-acid subsequences to determine individual confidence classifications for individual genomic coordinates or regions. Such coordinate-specific or region-specific confidence classifications can be further packaged into a newly augmented file or new file type—that is, a digital file with confidence classifications for genomic coordinates or regions (e.g., to supplement variant calls).

Beyond training a new type of machine-learning model, the disclosed systems can also apply the model to supplement or contextualize a variant call with empirically trained confidence classifications. After detecting a call variant at a genomic coordinate (or region) in a sample sequence, for instance, the disclosed systems can identify a coordinate-specific or region-specific confidence classification from a digital file for the genomic coordinate or region corresponding to the variant call. Based on the identified coordinate-specific or region-specific confidence classification, the disclosed systems can generate an indicator of the confidence classification for the genomic coordinate or region corresponding to the variant call for display on a graphical user interface. The disclosed systems can accordingly facilitate a graphical or textual indicator on a computing device specifying a confidence classification for a variant call at a genomic coordinate or region.

By training a genome-location-classification model as described herein, the disclosed systems create a first-of-its-kind machine-learning model to generate reportable ranges of confidence classifications for genomic coordinates or regions. Unlike the existing solutions that rely on confidence regions tied to a reference genome and untethered to empirical data from a sequencing pipeline, the disclosed genome-location-classification model can be both empirically trained and tailored to generate confidence classifications for a specific sequencing pipeline. Because the genome-location-classification model generates confidence classifications from an empirically trained process, the coordinate-or-region-specific confidence classifications from the genome-location-classification model give context and newfound accuracy to variant calls or other nucleobase calls.

### BRIEF DESCRIPTION OF THE DRAWINGS

The detailed description refers to the drawings briefly described below.

FIG. 1 illustrates a block diagram of a sequencing system including a genome-classification system in accordance with one or more embodiments.

FIG. 2 illustrates an overview of the genome-classification system training a machine-learning model to determine confidence classifications for genomic coordinates in accordance with one or more embodiments.

FIG. 3 illustrates an overview of the genome-classification system determining sequencing metrics with respect to a reference genome in accordance with one or more embodiments.

FIG. 4 illustrates an overview of a process in which the genome-classification system adjusts or prepares the sequencing metrics for input into a genome-location-classification model in accordance with one or more embodiments.

FIG. 5 illustrates a contextual nucleic-acid subsequence surrounding a nucleobase call in accordance with one or more embodiments.

FIG. 6A illustrates the genome-classification system training a machine-learning model to determine confidence classifications for genomic coordinates based on one or both of sequencing metrics and contextual nucleic-acid subsequences in accordance with one or more embodiments.

FIG. 6B illustrates the genome-classification system applying a trained version of a genome-location-classification model to determine confidence classifications for genomic coordinates based on one or both of sequencing metrics and contextual nucleic-acid subsequences in accordance with one or more embodiments.

FIG. 6C illustrates the sequencing system or the genome-classification system identifying and displaying confidence classifications from a genome-location-classification model corresponding to genomic coordinates of variant calls in accordance with one or more embodiments.

FIGS. 6D-6H illustrate the genome-classification system determining ground-truth classifications based on one or both of sequencing metrics for sample nucleic-acid sequences from genome samples and recall rates or precision rates for calling specific types of variants reflecting cancer or mosaicism based on an admixture of genome samples in accordance with one or more embodiments.

FIGS. 7A-7G illustrate graphs indicating informative sequencing metrics and sequencing-metric-derived data for genome-location-classification models in accordance with one or more embodiments.

FIG. 8 illustrates a graph depicting an accuracy with which the genome-location-classification model correctly determines confidence classifications for genomic coordinates based on sequencing metrics in accordance with one or more embodiments.

FIG. 9 illustrates a graph depicting an accuracy with which the genome-location-classification model correctly determines confidence classifications for genomic coordinates corresponding to different nucleotide variants based on contextual nucleic-acid subsequences in accordance with one or more embodiments.

FIGS. 10A-10B illustrate graphs depicting an accuracy with which the genome-location-classification model correctly determines confidence classifications for genomic coordinates corresponding to different nucleotide variants based on both sequencing metrics and contextual nucleic-acid subsequences in accordance with one or more embodiments.

FIGS. 11A-11B illustrate a flowchart of a series of acts for training a machine-learning model to determine confidence classifications for genomic coordinates in accordance with one or more embodiments.

FIG. 12 illustrates a flowchart of a series of acts for generating an indicator of a confidence classification for a genomic coordinate of a variant-nucleobase call from a digital file in accordance with one or more embodiments.

FIG. 13 illustrates a block diagram of an example computing device for implementing one or more embodiments of the present disclosure.

### DETAILED DESCRIPTION

This disclosure describes embodiments of a genome-classification system that trains a genome-location-classification model to determine labels or scores for genomic coordinates (or genomic regions) indicating the degree or extent to which nucleobases can be accurately identified at genomic coordinates or regions. To prepare inputs for the genome-location-classification model, the genome-classification system determines one or both of sequencing metrics for sample nucleic-acid sequences and contextual nucleic-acid subsequences surrounding particular nucleobase calls. In some cases, the genome-classification system determines such metrics and contextual nucleic-acid subsequences using a specific sequencing and bioinformatics pipeline. Accordingly, based on data derived or prepared from one or both of the sequencing metrics and contextual nucleic-acid subsequences—and by leveraging ground-truth classifications for genomic coordinates—the genome-classification system trains a genome-location-classification model to determine confidence classifications for genomic coordinates.

In certain implementations, the genome-classification system further determines confidence classifications for genomic coordinates (or regions) by providing data from sequencing metrics or contextual nucleic-acid subsequences corresponding to samples through the genome-location-classification model. The genome-classification system further encodes such coordinate-specific or region-specific confidence classifications into at least one digital file comprising confidence classifications for specific genomic coordinates or genomic regions. For example, the digital file may include annotations or other data indicators for genomic coordinates and/or genomic regions.

In addition or independent of training the genome-location-classification model, the genome-classification system can further determine confidence classifications for nucleobase calls (e.g., invariant calls or variant calls) based on the calls' particular genomic coordinates or region. Using data from a sequencing device, for instance, the genome-classification system determines a variant-nucleobase call or nucleobase-call invariant at a specific genomic coordinate (or specific region) in a sample nucleic-acid sequence. Such a nucleobase call may be determined using the same sequencing and bioinformatics pipeline as that used for training data to train the genome-location-classification model. The genome-classification system can then identify a confidence classification for the genomic coordinate or region corresponding to the nucleobase call (e.g., by accessing confidence classification data within a digital file generated by a trained genome-location-classification model). By identifying the confidence classification, the genome-classification system generates an indicator of the confidence classification for the genomic coordinate or region of a variant-nucleobase call or nucleobase-call invariant for display in a graphical user interface.

As noted in the preceding paragraphs, in some cases, the genome-classification system uses a single sequencing pipeline to determine nucleobase calls underlying sequencing metrics, contextual nucleic-acid subsequences, or variant-nucleobase calls. For instance, the genome-classification system may use a single sequencing pipeline with a same nucleic-acid-sequence-extraction method (e.g., extraction kit), a same sequencing device, and a same sequence-analysis software. Such a sequence-analysis software can include alignment software that aligns sequence reads with a reference genome and a variant caller software that identifies variant-nucleobase calls, such that a single sequencing pipeline uses a same alignment software and/or variant caller. By using a single sequencing pipeline, in certain implementations, the genome-classification system can both train and apply a genome-location-classification model that determines confidence classifications specific to the sequencing pipeline and increase the accuracy of those classifications for variant calls or other nucleobase calls by the pipeline.

To prepare data to input for training or applying the genome-location-classification model, in some embodiments, the genome-classification system determines sequencing metrics that include one or more of (i) alignment metrics for quantifying alignment of sample nucleic-acid sequences with genomic coordinates of an example nucleic-acid sequence (e.g., a reference genome or a nucleic-acid sequence from an ancestral haplotype), (ii) depth metrics for quantifying depth of nucleobase calls for sample nucleic-acid sequences at genomic coordinates of the example nucleic-acid sequence, or (iii) call-data-quality metrics for quantifying quality of nucleobase calls for sample nucleic-acid sequences at genomic coordinates of the example nucleic-acid sequence. For instance, the genome-classification system determines mapping-quality metrics, soft-clipping metrics, or other alignment metrics that measure an alignment of sample sequences with a reference genome. As another example, the genome-location-classification system determines forward-reverse-depth metrics (or other such depth metrics) or callability metrics for variant-nucleobase calls (or other such call-data-quality metrics).

In addition or in the alternative to using such sequencing metrics as data inputs for the genome-location-classification model, in certain cases, the genome-classification system determines contextual nucleic-acid subsequences surrounding a nucleobase call at a particular genomic coordinate. For instance, in some embodiments, the genome-classification system identifies, as a contextual nucleic-acid subsequence, the nucleobases from a reference genome (or from an ancestral haplotype sequence) located both upstream and downstream from an any nucleobase-call invariant or variant-nucleobase call, such as SNV, indel, structural variation, or a copy number variation (CNV). To illustrate, the genome-classification system may identify as a contextual nucleic-acid subsequence the fifty nucleobases upstream in a reference genome or ancestral haplotype sequence and the fifty nucleobases downstream from an SNV located at a particular genomic coordinate.

Regardless of whether the genome-classification system uses data derived from sequencing metrics or contextual nucleic-acid subsequences or both, the genome-classification system prepares the data as inputs for training a genome-location-classification model. In some cases, the genome-classification system trains a genome-location-classification model by determining projected confidence classifications for genomic coordinates and comparing the projected classifications to ground-truth classifications reflecting a Mendelian-inheritance pattern or a replicate concordance of nucleobase calls at a genomic coordinate. By using a loss function to compare the projected confidence classifications to ground-truth classifications for particular genomic coordinates, the genome-classification system can iteratively adjust parameters of the genome-location-classification model to more accurately determine confidence classifications.

As suggested above, the genome-location-classification model can output confidence classifications in various forms, including labels or scores. The genome-classification system may determine tiers of confidence levels including, for instance, a high-confidence classification, an intermediate-confidence classification, or a low-confidence classification indicating a degree to which nucleobase calls can be relied upon at a given genomic coordinate. Additionally or alternatively, the genome-classification system may determine a confidence score from a range of scores indicating a degree to which nucleobase calls can be relied upon at a given genomic coordinate.

After training and determining confidence classifications, the genome-classification system can generate or annotate one or more digital files to include confidence classifications specific to genomic coordinates. To give but one example, in some cases, the genome-classification system generates a modified version of a browser extensible data (BED) file comprising an annotation for each nucleobase call at a genomic coordinate identifying a corresponding confidence classification for the genomic coordinate. In some cases, the genome-classification system generates a BED file comprising annotations for genomic coordinates according to confidence-classification type, such as a BED file with annotations for genomic coordinates with high-confidence classifications, a BED file with annotations for genomic coordinates with intermediate-confidence classifications, and a BED file with annotations for genomic coordinates with low-confidence classifications. The genome-classification system may likewise generate a digital file with confidence classifications in Wiggle (WIG) format, Binary version of Sequence Alignment/Map (BAM) format, Variant Call File (VCF) format, Microarray format, or other digital-file formats. Upon identifying the relevant confidence classification for a nucleotide-call variant from a digital file, the genome-classification system may likewise provide an indicator of the classification for display on a graphical user interface. Such an indicator may be, for instance, a graphical indicator of a high-confidence, intermediate-confidence, or low-confidence classification (e.g., a color-coded graphical indicator).

As suggested above, the genome-classification system provides several technical benefits and technical improvements over conventional nucleic-acid-sequencing systems and corresponding sequencing-data-analysis software. For instance, the genome-classification system introduces a first-of-its-kind machine-learning model that is uniquely trained to perform a new application—generate confidence classifications for specific genomic coordinates at which nucleotide-variant calls or other nucleobases are determined. Unlike conventional variant callers or conventional reportable ranges that rely primarily on reference genome characteristics, the genome-classification system uses empirical data to train a genome-location-classification model to generate coordinate-specific or region-specific confidence classifications culminating in an empirical, reportable range of confidence classifications for nucleobase calls. A reportable range may include a variety of easy-to-understand labels, such as a high-confidence, intermediate-confidence, or low-confidence classifications—unlike the monolithic conventional classifications for reference genomes. In further contrast to the one-size-fits-all approach of existing sequencing systems that rely on confidence regions developed for a reference genome, in some embodiments, the genome-classification system can tailor the genome-location-classification model's confidence classifications to a single sequencing pipeline, thereby increasing the accuracy of confidence classifications for nucleobase calls from a particular sequencing device (and corresponding pipeline components) at the individual genomic-coordinate level.

In addition to introducing a first-of-its-kind machine-learning model, compared to existing sequencing systems, the genomic-classification system improves the accuracy and breadth of determining a confidence level for nucleobase calls at specific genomic coordinates—across a genome. For instance, the genome-classification system increases the precision, recall, and concordance with which a sequencing system accurately identifies variants at genomic coordinates. In some implementations, a sequencing system accurately identifies SNVs with approximately 99.9% precision, 99.9% recall, and 99.9% concordance—at genomic coordinates labeled with a high-confidence classification by a disclosed genome-location-classification model for about 90.3% of the reference genome. This disclosure reports additional statistics for precision, recall, and concordance below. In contrast to the accuracy and breadth of the disclosed genome-classification system, GIAB or GA4GH's conventional reportable ranges (with a single classification) for a reference genome are limited to about 79-84% of the reference genome. Further, Platinum Genomes excludes problematic genomic regions that the genome-classification can now classify with exceptional precision, recall, and concordance.

In addition to improved accuracy, in certain embodiments, the genome-classification system improves flexibility over conventional methods by reliably determining confidence classifications for different variant types at specific genomic coordinates. As noted above, conventional reportable ranges developed by GIAB and GA4GH do not distinguish between variant types. By contrast, in some implementations, the genome-classification system determines confidence classifications for genomic coordinates specific to a variant type (e.g., SNVs, indels, variant-nucleobase calls reflecting cancer or mosaicism). For instance, the genome-location-classification model may generate different confidence classifications for genomic coordinates at which a single nucleotide variant, a nucleobase insertion, a nucleobase deletion, a part of a structural variation, or a part of a CNV is detected. Accordingly, a confidence classification from the genome-location-classification model can indicate a specific degree of confidence that a single nucleotide variant can be accurately determined at particular genomic coordinates—as opposed to confidence classifications that may differ for a nucleobase insertion, a nucleobase deletion, a part of a structural variation, or a part of a CNV.

Independent of improved accuracy or flexibility, in some cases, the genome-classification system generates a new file type or newly augmented file type that introduces specific confidence classifications for specific genomic coordinates or regions—unlike conventional genomic files. By way of background, a conventional BED file often includes fields for a name of a chromosome (e.g., chrom=chr3, chrY), a starting position for a nucleobase or feature for the chromosome (e.g., chromStart=0 for first base number), and an ending position for a feature (e.g., chromEnd=100). In some cases, a BED file also includes fields to identify specific genes and identify a detected variant. Like a WIG file, BAM file, VSF file, or a Microarray file, a conventional BED file has no field or annotation for confidence classifications for specific genomic coordinates. By contrast, the genome-classification system generates a new digital file with an annotation or other indicator of confidence classifications for specific genomic coordinates or regions in BED, BAM, WIG, VCF, Microarray, or other digital file formats. As noted above, in some cases, the genome-classification system generates different digital files each comprising annotations for genomic coordinates according to different confidence-classification types (e.g., a different digital file for each of high-confidence classifications, intermediate-confidence classifications, low-confidence classifications). By introducing the new confidence-classification indicators, the genome-classification system can provide a specific confidence classification in label or score form for a variety of different variant-nucleobase calls at specific genomic coordinates or regions.

As indicated by the foregoing description, this disclosure describes various features and advantages of the genome-classification system. As used in this disclosure, for instance, the term “sample nucleic-acid sequence” or “sample sequence” refers to a sequence of nucleotides isolated or extracted from a sample organism (or a copy of such an isolated or extracted sequence). In particular, a sample nucleic-acid sequence includes a segment of a nucleic-acid polymer that is isolated or extracted from a sample organism and composed of nitrogenous heterocyclic bases. For example, a sample nucleic-acid sequence can include a segment of deoxyribonucleic acid (DNA), ribonucleic acid (RNA), or other polymeric forms of nucleic acids or chimeric or hybrid forms of nucleic acids noted below. More specifically, in some cases, the sample nucleic-acid sequence is found in a sample prepared or isolated by a kit and received by a sequencing device.

As further used herein, the term “nucleobase call” refers to an assignment or determination of a particular nucleobase to add to an oligonucleotide for a sequencing cycle. In particular, a nucleobase call indicates an assignment or a determination of the type of nucleotide that has been incorporated within an oligonucleotide on a nucleotide-sample slide. In some cases, a nucleobase call includes an assignment or determination of a nucleobase to intensity values resulting from fluorescent-tagged nucleotides added to an oligonucleotide of a nucleotide-sample slide (e.g., in a well of a flow cell). Alternatively, a nucleobase call includes an assignment or determination of a nucleobase to chromatogram peaks or electrical current changes resulting from nucleotides passing through a nanopore of a nucleotide-sample slide. By using nucleobase calls, a sequencing system determines a sequence of a nucleic-acid polymer. For example, a single nucleobase call can comprise an adenine call, a cytosine call, a guanine call, or a thymine call for DNA (abbreviated as A, C, G, T) or a uracil call (instead of a thymine call) for RNA (abbreviated as U).

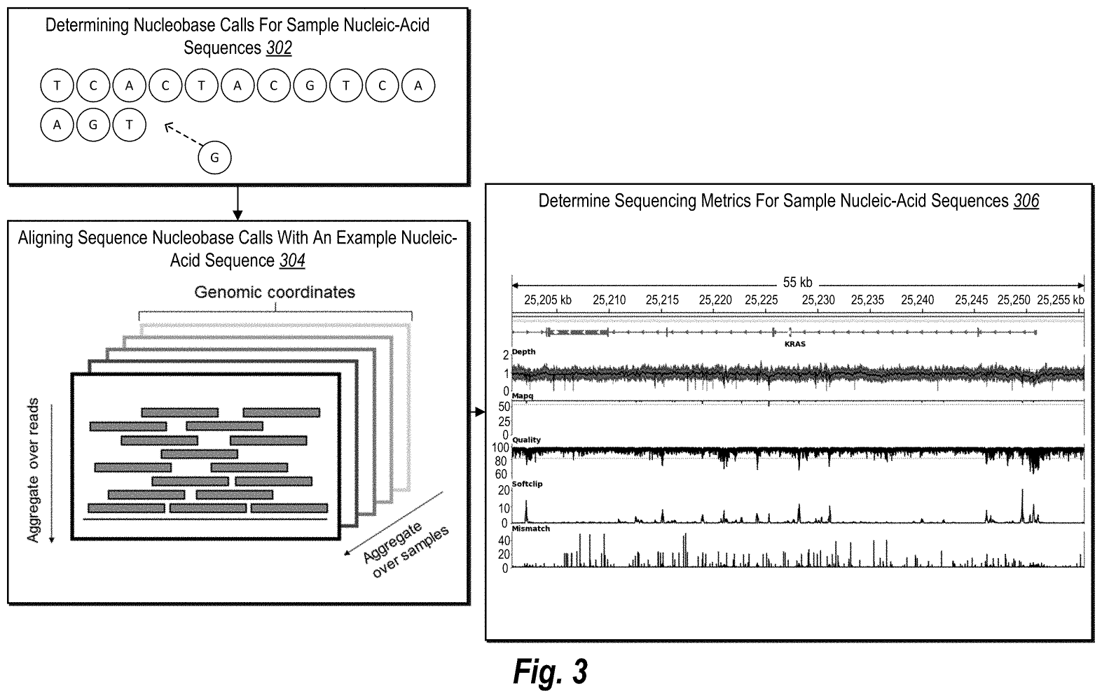

As noted above, in some embodiments, the genome-classification system determines sequencing metrics for comparing sample nucleic-acid sequences with an example nucleic-acid sequence (e.g., a reference genome or a nucleic-acid sequence from an ancestral haplotype). As used herein, the term “sequencing metrics” refers to a quantitative measurement or score indicating a degree to which individual nucleobase calls (or a sequence of nucleobase calls) align, compare, or quantify with respect to a genomic coordinate or genomic region of an example nucleic-acid sequence. In particular, sequencing metrics can include alignment metrics that quantify a degree to which sample nucleic-acid sequences align with genomic coordinates of an example nucleic-acid sequence, such as deletion-size metrics or mapping-quality metrics. Further, sequencing metrics can include depth metrics that quantify the depth of nucleobase calls for sample nucleic-acid sequences at genomic coordinates of an example nucleic-acid sequence, such as forward-reverse-depth metrics or normalized-depth metrics. Sequencing metrics can also include call-data-quality metrics that quantify a quality or accuracy of nucleobase calls, such as nucleobase-call-quality metrics, callability metrics, or somatic-quality metrics. In some embodiments, data derived or prepared from the sequencing metrics can be input into a genome-location-classification model. This disclosure further describes sequencing metrics and provides additional examples below with reference to FIG. 3.

As noted above, in some embodiments, the genome-classification system can determine a contextual nucleic-acid subsequence surrounding a nucleobase call at a genomic coordinate. As used herein, the term “contextual nucleic-acid subsequence” refers to a series of nucleobases from an example nucleic-acid sequence that surround (e.g., flank on each side or neighbor) a genomic coordinate for a particular nucleobase call in a sample nucleic-acid sequence. In some examples, a contextual nucleic-acid subsequence refers to a series of nucleobases from a reference sequence (or from a genome or sequence of an ancestral haplotype) that surround a nucleotide-variant call or an invariant call in a sample nucleic-acid sequence. In particular, a contextual nucleic-acid subsequence includes nucleobases from an example nucleic-acid sequence that are (i) located both upstream and downstream from a genomic coordinate(s) for a particular nucleobase call(s) of a sample nucleic-acid sequence and (ii) within a threshold number of genomic coordinates from the genomic coordinate(s) for the particular nucleobase call(s). Accordingly, a contextual nucleic-acid subsequence may include the fifty nucleobases upstream in an example nucleic-acid sequence (e.g., reference genome) and the nucleobases of the fifty nucleobases downstream from an SNV located at a particular genomic coordinate.

As just noted, the genome-classification system can determine a contextual nucleic-acid subsequence from an example nucleic-acid sequence. As used herein, the term “example nucleic-acid sequence” refers to a sequence of nucleotides from a reference or related genome, such as a reference genome or a sequence of an ancestral haplotype. In particular, an example nucleic-acid sequence includes a segment of a nucleic-acid sequence inherited from a sample's ancestor (e.g., ancestral haplotype) or of a digital nucleic-acid sequence (e.g., reference genome). In some cases, an ancestral haplotype sequence comes from a parent or grandparent of a sample.

As further used herein, the term “genomic coordinate” refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

As mentioned above, a “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870).

As noted above, a genomic coordinate includes a position within a reference genome. Such a position may be within a particular reference genome. As used herein, the term “reference genome” refers to a digital nucleic-acid sequence assembled as a representative example of genes for an organism. Regardless of the sequence length, in some cases, a reference genome represents an example set of genes or a set of nucleic-acid sequences in a digital nucleic-acid sequenced determined by scientists as representative of an organism of a particular species. For example, a linear human reference genome may be GRCh38 or other versions of reference genomes from the Genome Reference Consortium. As a further example, a reference genome may include a reference graph genome that includes both a linear reference genome and paths representing nucleic-acid sequences from ancestral haplotypes, such as Illumina DRAGEN Graph Reference Genome hg19.

As used herein, the term “genome-location-classification model” refers to a machine-learning model trained to generate confidence classifications for genomic coordinates or genomic regions. Accordingly, a genome-location-classification model can include a statistical machine-learning model or a neural network trained to generate such confidence classifications. In some cases, for example, the genome-location-classification model takes the form of a logistic regression model, a random forest classifier, or a convolutional neural network (CNN). But other machine-learning models may be trained or used.

As just suggested, a genome-location-classification model may be a genome-location-classification-neural network. A neural network includes a model of interconnected artificial neurons (e.g., organized in layers) that communicate and learn to approximate complex functions and generate outputs (e.g., generated digital images) based on a plurality of inputs provided to the neural network. In some cases, a neural network refers to an algorithm (or set of algorithms) that implements deep learning techniques to model high-level abstractions in data.

Regardless of the form, a genome-location-classification model generates confidence classifications. As used herein, the term “confidence classification” refers to a label, score, or metric indicating a confidence or reliability with which nucleobases can be determined or detected at genomic coordinates or genomic regions. In particular, a confidence classification includes a label, score, or metric classifying a degree to which nucleobases can be accurately called for particular genomic coordinates or within particular genomic regions. For instance, in certain implementations, a confidence classification includes labels identifying a high-confidence classification, an intermediate-confidence classification, or a low-confidence classification for a genomic coordinate. Additionally or alternatively, a confidence classification includes a score indicating a probability or likelihood that a nucleobase can be accurately determined at a genomic coordinate.

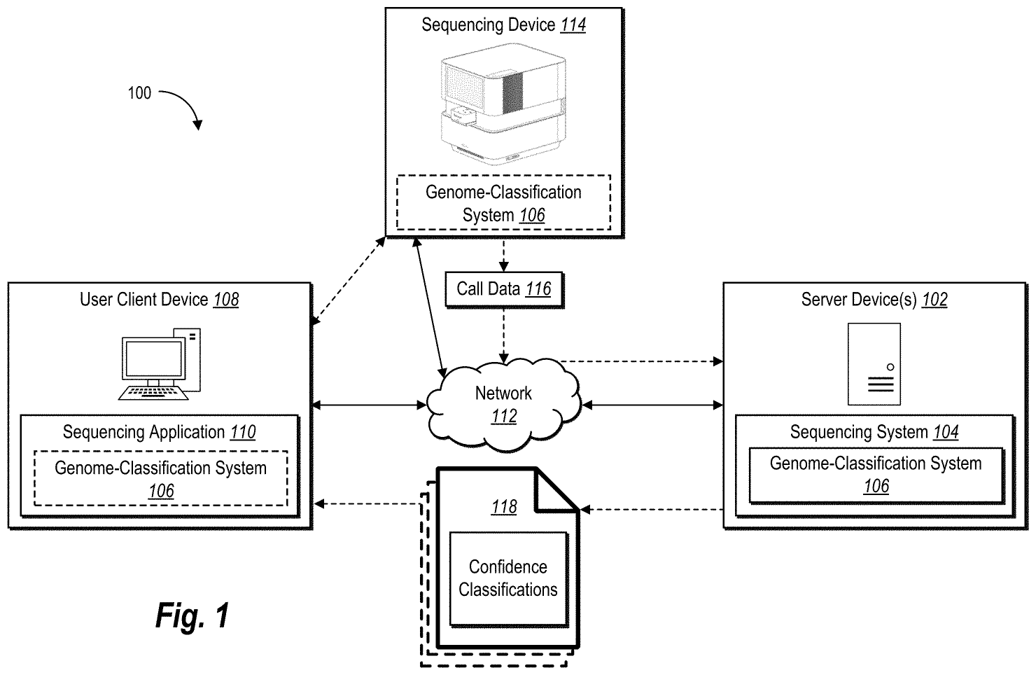

The following paragraphs describe the genome-classification system with respect to illustrative figures that portray example embodiments and implementations. For example, FIG. 1 illustrates a schematic diagram of a system environment (or “environment”)100 in which a genome-classification system 106 operates in accordance with one or more embodiments. As illustrated, the environment 100 includes one or more server device(s) 102 connected to a user client device 108 and a sequencing device 114 via a network 112. While FIG. 1 shows an embodiment of the genome-classification system 106, this disclosure describes alternative embodiments and configurations below.

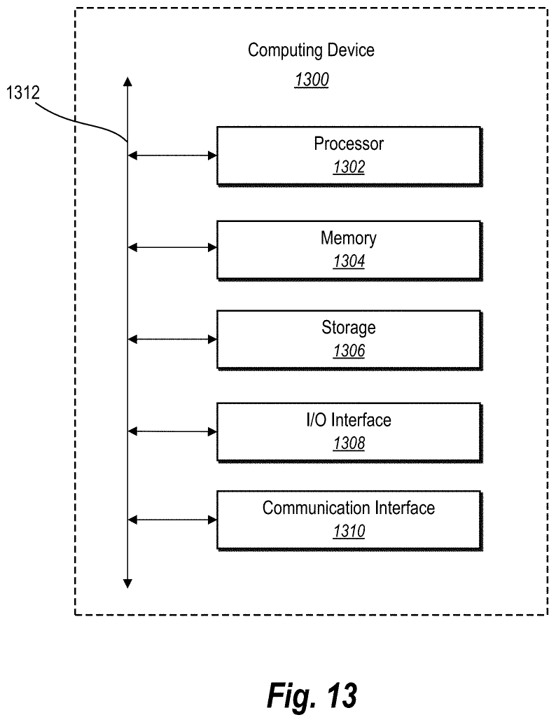

As shown in FIG. 1, the server device(s) 102, the user client device 108, and the sequencing device 114 are connected via the network 112. Accordingly, each of the components of the environment 100 can communicate via the network 112. The network 112 comprises any suitable network over which computing devices can communicate. Example networks are discussed in additional detail below with respect to FIG. 13.

As indicated by FIG. 1, the sequencing device 114 comprises a device for sequencing a nucleic-acid polymer. In some embodiments, the sequencing device 114 analyzes nucleic-acid segments or oligonucleotides extracted from samples to generate data utilizing computer implemented methods and systems (described herein) either directly or indirectly on the sequencing device 114. More particularly, the sequencing device 114 receives and analyzes, within nucleotide-sample slides (e.g., flow cells), nucleic-acid sequences extracted from samples. In one or more embodiments, the sequencing device 114 utilizes SBS to sequence nucleic-acid polymers. In addition or in the alternative to communicating across the network 112, in some embodiments, the sequencing device 114 bypasses the network 112 and communicates directly with the user client device 108.

As further indicated by FIG. 1, the server device(s) 102 may generate, receive, analyze, store, and transmit digital data, such as data for determining nucleobase calls or sequencing nucleic-acid polymers. As shown in FIG. 1, the sequencing device 114 may send (and the server device(s) 102 may receive) call data 116 from the sequencing device 114. The server device(s) 102 may also communicate with the user client device 108. In particular, the server device(s) 102 can send to the user client device 108 a digital file 118 comprising confidence classifications for genomic coordinates. As indicated by FIG. 1, in some embodiments, the server device(s) 102 send separate digital files each comprising different confidence classifications (e.g., a different digital file for each of high-confidence classifications, intermediate-confidence classifications, low-confidence classifications). In some cases, the digital file 118 (and/or the other digital files) also includes nucleobase calls, error data, and other information.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server.

As further shown in FIG. 1, the server device(s) 102 can include a sequencing system 104. Generally, the sequencing system 104 analyzes the call data 116 received from the sequencing device 114 to determine nucleobase sequences for nucleic-acid polymers. For example, the sequencing system 104 can receive raw data from the sequencing device 114 and determine a nucleobase sequence for a nucleic-acid segment. In some embodiments, the sequencing system 104 determines the sequences of nucleobases in DNA and/or RNA segments or oligonucleotides. In addition to processing and determining sequences for nucleic-acid polymers, the sequencing system 104 also generates the digital file 118 comprising confidence classifications and can send the digital file 118 to the user client device 108.

As just mentioned, and as illustrated in FIG. 1, the genome-classification system 106 analyzes the call data 116 from the sequencing device 114 to determine nucleobase calls for sample nucleic-acid sequences. In some embodiments, the genome-classification system 106 determines one or both of sequencing metrics for such sample nucleic-acid sequences and contextual nucleic-acid subsequences around particular nucleobase calls. Based on data derived or prepared from one or both of the sequencing metrics and the contextual nucleic-acid subsequences—and ground-truth classifications for genomic coordinates—the genome-classification system 106 trains a genome-location-classification model to determine confidence classifications for genomic coordinates. The genome-classification system 106 further determines a set of confidence classifications for a set of genomic coordinates (or regions) by providing data prepared from (i) a set of sequencing metrics corresponding to samples or (ii) contextual nucleic-acid subsequences corresponding to samples to the genome-location-classification model as inputs. Based on these inputs, for example, the genome-classification system 106 uses the genome-location-classification model to determine confidence classifications for each genomic coordinate of a reference genome. As noted above, the genome-classification system 106 further generates a digital file comprising confidence classifications for the set of genomic coordinates or regions.

As further illustrated and indicated in FIG. 1, the user client device 108 can generate, store, receive, and send digital data. In particular, the user client device 108 can receive the call data 116 from the sequencing device 114. Furthermore, the user client device 108 may communicate with the server device(s) 102 to receive the digital file 118 comprising nucleobase calls and/or confidence classifications. The user client device 108 can accordingly present confidence classifications for genomic coordinates—sometimes along with nucleotide-variant calls or nucleotide-invariant calls—within a graphical user interface to a user associated with the user client device 108.

The user client device 108 illustrated in FIG. 1 may comprise various types of client devices. For example, in some embodiments, the user client device 108 includes non-mobile devices, such as desktop computers or servers, or other types of client devices. In yet other embodiments, the user client device 108 includes mobile devices, such as laptops, tablets, mobile telephones, or smartphones. Additional details with regard to the user client device 108 are discussed below with respect to FIG. 13.

As further illustrated in FIG. 1, the user client device 108 includes a sequencing application 110. The sequencing application 110 may be a web application or a native application stored and executed on the user client device 108 (e.g., a mobile application, desktop application). The sequencing application 110 can receive data from the genome-classification system 106 and present, for display at the user client device 108, data from the digital file 118(e.g., by presenting particular confidence classifications by genomic coordinate). Furthermore, the sequencing application 110 can instruct the user client device 108 to display an indicator of a confidence classification for a genomic coordinate of a variant-nucleobase call or a nucleobase-call invariant.

As further illustrated in FIG. 1, the genome-classification system 106 may be located on the user client device 108 as part of the sequencing application 110 or on the sequencing device 114. Accordingly, in some embodiments, the genome-classification system 106 is implemented by (e.g., located entirely or in part) on the user client device 108. In yet other embodiments, the genome-classification system 106 is implemented by one or more other components of the environment 100, such as the sequencing device 114. In particular, the genome-classification system 106 can be implemented in a variety of different ways across the server device(s) 102, the network 112, the user client device 108, and the sequencing device 114.

Though FIG. 1 illustrates the components of environment 100 communicating via the network 112, in certain implementations, the components of environment 100 can also communicate directly with each other, bypassing the network. For instance, and as previously mentioned, in some implementations, the user client device 108 communicates directly with the sequencing device 114. Additionally, in some embodiments, the user client device 108 communicates directly with the genome-classification system 106. Moreover, the genome-classification system 106 can access one or more databases housed on or accessed by the server device(s) 102 or elsewhere in the environment 100.

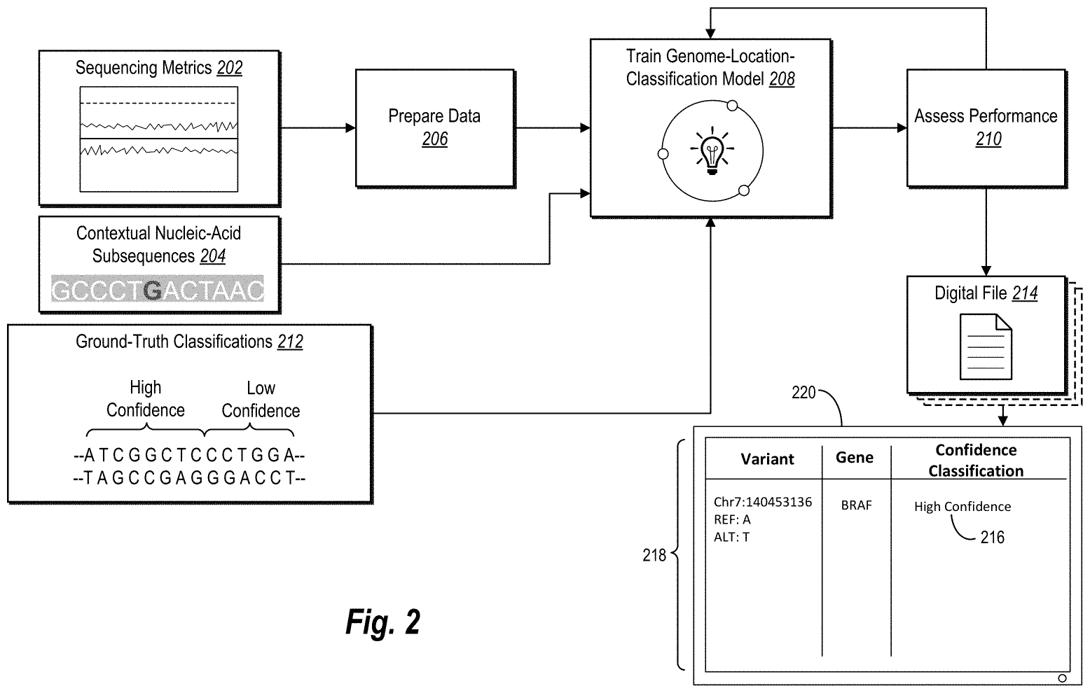

As indicated above, the genome-classification system 106 trains a genome-location-classification model to determine confidence classifications for genomic coordinates or genomic regions. FIG. 2 illustrates an overview of the genome-classification system 106 using one or both of sequencing metrics and contextual nucleic-acid subsequences to train a genome-location-classification model 208. As described further below, the genome-classification system 106 determines one or both of sequencing metrics 202 and contextual nucleic-acid subsequences 204 for sample nucleic-acid sequences. Based on data derived or prepared from one or more of the sequencing metrics 202 or the contextual nucleic-acid subsequences 204, the genome-classification system 106 trains the genome-location-classification model 208 to generate confidence classifications for genomic coordinates. After training and testing the genome-location-classification model 208, the genome-classification system 106 generates a digital file 214 comprising confidence classifications for particular genomic coordinates and can cause a computing device 220 to display such confidence classifications from the digital file 214.

As shown in FIG. 2, for example, the genome-classification system 106 optionally determines the sequencing metrics 202 for comparing sample nucleic-acid sequences with genomic coordinates of an example nucleic-acid sequence (e.g., a reference genome or a nucleic-acid sequence from an ancestral haplotype). In preparation for determining the sequencing metrics 202, in some cases, the sequencing system 104 or the genome-classification system 106 receives call data and determines nucleobase calls for nucleic-acid sequences extracted from a diverse cohort of samples. In some cases, for instance, the genome-classification system 106 uses nucleobase calls and nucleic-acid sequences determined from 30-150 samples across different populations. To extract and determine nucleobase calls for each sample nucleic-acid sequence, in certain implementations, the genome-classification system 106 uses a common or a single sequencing pipeline—including the same nucleic-acid-sequence-extraction method, sequencing device, and sequence-analysis software for each sample.

Based on the nucleobase calls within the sample nucleic-acid sequences, the genome-classification system 106 determines the sequencing metrics 202. As indicated above, the sequencing metrics 202 can include one or more of (i) alignment metrics that quantify a degree to which the sample nucleic-acid sequences align with an example nucleic-acid sequence (e.g., a reference genome or a nucleic-acid sequence of an ancestral haplotype), (ii) depth metrics that quantify the depth of nucleobase calls for sample nucleic-acid sequences at genomic coordinates of an example nucleic-acid sequence, or (iii) call-data-quality metrics that quantify a quality or accuracy of nucleobase calls of the example nucleic-acid sequence. When determining alignment metrics, for instance, the genome-classification system 106 determines one or more of deletion-entropy metrics, deletion-size metrics, mapping-quality metrics, positive-insert-size metrics, negative-insert-size metrics, soft-clipping metrics, read-position metrics, or read-reference-mismatch metrics for sample nucleic-acid sequences. When determining depth metrics, by contrast, the genome-classification system 106 determines one or more of forward-reverse-depth metrics, normalized-depth metrics, depth-under metrics, depth-over metrics, or peak-count metrics. When determining call-data-quality metrics, for instance, the genome-classification system 106 determines one or more of nucleobase-call-quality metrics, callability metrics, or somatic-quality metrics for the sample nucleic-acid sequences. Sequencing metrics 202 are described further below with respect to FIG. 3.

In addition to determining the sequencing metrics 202, as shown in FIG. 2, the genome-classification system 106 further prepares data 206 from the sequencing metrics 202 for input into the genome-location-classification model 208. When preparing the data for input, the genome-classification system 106 can extract data from the sequencing metrics 202 by summarizing or averaging the sequencing metrics 202 in a variety of ways. In addition to extraction, in certain cases, the genome-classification system 106 also modifies the sequencing metrics 202 or the extracted data from the sequencing metrics 202 to format the data for input into the genome-location-classification model 208. After or in addition to extracting and modifying the sequencing metrics 202, in some embodiments, the genome-classification system 106 further standardizes the different types of the sequencing metrics 202 to a same scale (e.g., with a mean of 0 and a standard deviation of 1).

As further shown in FIG. 2, in addition or in the alternative to determining the sequencing metrics 202, the genome-classification system 106 determines the contextual nucleic-acid subsequences 204—from an example nucleic-acid sequence (e.g., a reference genome or ancestral haplotype sequence)—that surround a nucleobase call at a particular genomic coordinate. For each such contextual nucleic-acid subsequence, in some cases, the genome-classification system 106 determines both the upstream and downstream nucleobases in a reference genome that are within a threshold coordinate distance from a genomic coordinate for a particular nucleobase call or from genomic coordinates for particular nucleobase calls. For example, the genome-classification system 106 can determine the upstream and downstream nucleobases within twenty, fifty, a hundred, or a different number of nucleobases from a genomic coordinate for an SNV, indel, structural variant, CNV, or other variant.

As further explained below, the contextual nucleic-acid subsequences 204 can include or exclude the nucleobase call(s) for the genomic coordinate(s) corresponding to the particular SNV, indel, structural variant, CNV, or other variant type at issue. Additionally, in certain implementations, the genome-classification system 106 derives or prepares data from the contextual nucleic-acid subsequences 204 by, for instance, applying a vector algorithm to package or condense the contextual nucleic-acid subsequences 204 into a format for input into the genome-location-classification model 208.

Having determined one or both of data prepared from the sequencing metrics 202 and the contextual nucleic-acid subsequences 204, the genome-classification system 106 trains the genome-location-classification model 208 based on such data. For example, the genome-classification system 106 iteratively inputs one or both of the data prepared from the sequencing metrics 202 and the contextual nucleic-acid subsequences 204—along with an indicator of the corresponding genomic coordinate or region—into the genome-location-classification model 208. Based on the iterative input, the genome-location-classification model 208 generates a projected confidence classification for each corresponding genomic coordinate or genomic region.

Upon generating the projected confidence classification, the genome-classification system 106 assesses the performance 210 of the genome-location-classification model 208 using projected confidence classifications in training iterations. For instance, the genome-classification system 106 compares the projected confidence classification with a ground-truth classification from the ground-truth classifications 212 for the corresponding genomic coordinate or genomic region. In each training iteration, for instance, the genome-classification system 106 executes a loss function to determine a loss between the predicted confidence classification for a genomic coordinate and a ground-truth classification for the genomic coordinate. Based on the determined loss, the genome-classification system 106 adjusts one or more parameters of the genome-location-classification model 208 to improve the accuracy with which the genome-location-classification model 208 generates projected confidence classifications. By iteratively executing such training iterations, the genome-classification system 106 trains the genome-location-classification model 208 to determine confidence classifications.

After training the genome-location-classification model 208, in some embodiments, the genome-classification system 106 uses a trained version of the genome-location-classification model 208 to determine a set of confidence classifications for a set of genomic coordinates (or regions)—based on a set of sequencing metrics and/or a set of contextual nucleic-acid subsequences. In some embodiments, the genome-classification system 106 determines the set of sequencing metrics and/or the set of contextual nucleic-acid subsequences from different samples. By determining a confidence classification for each genomic coordinate or region—or for at least a subset of genomic coordinates or regions corresponding to a reference genome—the genome-classification system 106 generates a coordinate-specific or region-specific classification indicating whether nucleobases can be accurately detected at such genomic coordinates or regions. Because the nucleobase calls upon which the sequencing metrics 202 or the contextual nucleic-acid subsequences 204 are determined use a single or defined sequencing pipeline, the genome-classification system 106 can likewise determine confidence classifications for genomic coordinates or regions based on sample nucleic-acid sequences that are analyzed using the same defined sequencing pipeline.

As further shown in FIG. 2, the genome-classification system 106 generates a digital file 214 comprising the confidence classifications for the genomic coordinates or regions. In some cases, the digital file 214 includes the confidence classifications as a reference file that computing devices can access to identify confidence classifications for particular genomic coordinates or regions. The digital file 214 (or a set of digital files) can include a confidence classification of high confidence, intermediate confidence, or low confidence—or a confidence score—for each genomic coordinate. Additionally, in some cases, the genome-classification system 106 nucleobase calls in the digital file 214 for orthogonal validation using a different sequencing method because the nucleobase calls are located at genomic coordinates corresponding to a confidence classification of lower reliability (e.g., low-confidence classification or below a confidence-score threshold).

As explained further below, in certain cases, the digital file 214 includes nucleotide-variant calls for particular genomic coordinates and the confidence classifications for the particular genomic coordinates. In such cases, the digital file 214 provides context for the reliability with which a clinician or patient may rely on nucleobase calls, including nucleotide-variant calls. As further indicated by FIG. 2, in some embodiments, the genome-classification system 106 generates separate digital files that each comprise different confidence classifications (e.g., a different digital file for each of high-confidence classifications, intermediate-confidence classifications, low-confidence classifications).

In addition to generating the digital file 214 and as further shown in FIG. 2, in some embodiments, the genome-classification system 106 further provides to the computing device 220 a confidence indicator 216 of a particular confidence classification for a genomic coordinate of a nucleobase call, such as a variant-nucleobase call or a nucleobase-call invariant. As indicated by FIG. 2, the genome-classification system 106 can integrate the confidence classification not only into the digital file 214 but also into data for reporting variant calls or invariant calls on a graphical user interface 218 of the computing device 220. For example, as depicted in FIG. 2, the sequencing system 104 or the genome-classification system 106 provides the confidence indicator 216 for display within the graphical user interface 218 along with a genomic coordinate for a variant call and an identifier for a particular gene. The sequencing system 104 or the genome-classification system 106 can likewise provide a confidence indicator for an invariant call for display on a graphical user interface along with the same or similar genomic-coordinate and/or gene information.

As noted above, the genome-classification system 106 determines sequencing metrics for comparing sample nucleic-acid sequences with genomic coordinates of a reference genome. In accordance with one or more embodiments, FIG. 3 illustrates the genome-classification system 106 determining nucleobase calls for sample nucleic-acid sequences 302, aligning sequence nucleobase calls with an example nucleic-acid sequence 304, and determining sequencing metrics for the sample nucleic-acid sequences 306. As described below, the genome-classification system 106 determines nucleobase calls, aligns sample nucleic-acid sequences, and determines sequencing metrics for specific genomic coordinates within a reference genome.

As shown in FIG. 3, for instance, the genome-classification system 106 determines nucleobase calls for sample nucleic-acid sequences 302. In preparation for such nucleobase calls, in some embodiments, nucleic-acid sequences are extracted or isolated from samples of diverse ethnicities using an extraction kit or specific nucleic-acid-sequence-extraction method. After extraction, the sequencing device 114 uses SBS sequencing or Sanger sequencing to synthesize copies and reverse strands for the sample nucleic-acid sequences and generate call data indicating the individual nucleobases incorporated into growing nucleic-acid sequences. Based on the call data, the sequencing system 104 determines nucleobase calls within the nucleic-acid sequences.

In some embodiments, a single or defined pipeline processes and determines the nucleobases of such nucleic-acid sequences for each sample. For instance, the sequencing system 104 may use a single sequencing pipeline comprising a same nucleic-acid-sequence-extraction method (e.g., extraction kit), a same sequencing device, and a same sequence-analysis software. In particular, a single pipeline may include, for instance, extracting DNA segments using Illumina Inc.'s TruSeq PCR-Free sample preparation kit for the nucleic-acid-sequence-extraction method; sequencing using a NovaSeq 6000 Xp, NextSeq 550, NextSeq 1000, or NextSeq 2000 for the sequencing device; and determining nucleobase calls using Dragen Germline Pipeline for the sequence-analysis software.

After determining nucleobase calls for the sample nucleic-acid sequences, as further shown in FIG. 3, the genome-classification system 106 aligns sequence nucleobase calls with an example nucleic-acid sequence 304. For instance, the sequencing system 104 or the genome-classification system 106 approximately matches the nucleobases of particular nucleic-acid sequences (over various reads) with the nucleobases of a reference genome (e.g., a linear reference genome or a graph reference genome). As indicated by FIG. 3, the genome-classification system 106 repeats the alignment process for the nucleic-acid sequences from each sample. As indicated above, in addition or in the alternative to aligning nucleobase calls with a reference genome, in some cases, aligns nucleobase calls (e.g., from nucleotide reads) with one or more nucleic-acid sequences from ancestral haplotypes. Once approximately aligned, the genome-classification system 106 can identify the nucleobase calls at particular genomic coordinates of the reference genome for each sample.

As suggested by FIG. 3, in some implementations, the sequencing system 104 or the genome-classification system 106 aligns sequence nucleobase calls with the example nucleic-acid sequence 304—and aggregates read and sample data for such nucleobase calls—as part of generating one or both of BAM and VCF files. To do so, the sequencing system 104 or the genome-classification system 106 generates, for each sample, a BAM file comprising data for aligned sample nucleic-acid sequences and a VCF file comprising data for nucleic-variant calls at genomic coordinates of the reference genome.

As further shown in FIG. 3, after determining nucleobase calls and aligning sample nucleic-acid sequences, the genome-classification system 106 determines sequencing metrics for the sample nucleic-acid sequences 306. In some embodiments, the genome-classification system 106 determines sequencing metrics for the sample nucleic-acid sequences at each genomic coordinate (or each genomic region). As indicated above, the genome-classification system 106 optionally determines the sequencing metrics from BAM and VCF files for the various samples. As explained below, the genome-classification system 106 determines one or more sequencing metrics quantifying depth, alignment, or call-data quality at a genomic coordinate. The following paragraphs describe example sequencing metrics as roughly grouped according to alignment, depth, and call-data quality.

As just indicated, the genome-classification system 106 can determine alignment metrics that quantify alignment of nucleobase calls for sample nucleic-acid sequences with genomic coordinates of an example nucleic-acid sequence (e.g., a reference genome or a nucleic-acid sequence of an ancestral haplotype). To illustrate, in some cases, the genome-classification system 106 determines mapping-quality metrics for sample nucleic-acid sequences by, for instance, determining a mean or median mapping quality of reads at a genomic coordinate. In some such embodiments, the genome-classification system 106 identifies or generates mapping quality (MAPQ) scores for nucleobase calls at genomic coordinates, where a MAPQ score represents—10 log10 Pr{mapping position is wrong}, rounded to the nearest integer. In the alternative to a mean or median mapping quality, in some embodiments, the genome-classification system 106 determines mapping-quality metrics for sample nucleic-acid sequences by determining a full distribution of mapping qualities for all reads aligning with a genomic coordinate or an ancestral haplotype. In addition or in the alternative to mapping-quality metrics, the genome-classification system 106 can determine soft-clipping metrics for sample nucleic-acid sequences by, for instance, determining a total number of soft-clipped nucleobases spanning a genomic coordinate corresponding to a reference genome or an ancestral haplotype. Accordingly, in some cases, the genome-classification system 106 determines a number of nucleobases that do not match an example nucleic-acid sequence (e.g., a reference genome or an ancestral haplotype) at particular genomic coordinates on either side of a read (e.g., 5 prime end or 3 prime end of a read) and are ignored for purposes of alignment.

As a further example of alignment metrics, in some embodiments, the genome-classification system 106 determines read-reference-mismatch metrics for sample nucleic-acid sequences by, for instance, determining a total number of nucleobases that do not match a nucleobase of an example nucleic-acid sequence (e.g., a reference genome or ancestral haplotype) at a particular genomic coordinate across multiple reads (e.g., all reads overlapping the particular genomic coordinate) or across multiple cycles (e.g., all cycles). By contrast, in certain cases, the genome-classification system 106 determines read-position metrics for sample nucleic-acid sequences by, for example, determining a mean or median position within a sequencing read of nucleobases covering a genomic coordinate.

In addition to the alignment metrics noted above, the genome-classification system 106 can determine alignment by determining indel metrics that quantify indels at genomic coordinates for sample nucleic-acid sequences, such as deletion metrics. In some cases, the genome-classification system 106 determines deletion-size metrics for sample nucleic-acid sequences by, for instance, determining a mean or median size of deletions spanning a genomic coordinate of a reference genome. Further, in certain implementations, the genome-classification system 106 determines deletion-entropy metrics for sample nucleic-acid sequences by, for instance, determining a distribution or variance of deletion size for a genomic coordinate or genomic region of a reference genome. A genomic coordinate or region with consistent or repeated deletions in sample nucleic-acid sequences of a single nucleobase (e.g., 20% of samples include a single nucleobase deletion) has less deletion entropy than a different genomic coordinate or region with varying deletion size in sample nucleic-acid sequences (e.g., 20% of samples include either a single-nucleobase deletion, 5-nucleobase deletion, or 10-nucleobase deletion).

In addition to deletion metrics as examples of alignment metrics noted above, the genome-classification system 106 can determine insertion-size metrics that quantify insertions at genomic coordinates for sample nucleic-acid sequences. For instance, in certain implementations, the genome-classification system 106 determines positive-insert-size metrics for sample nucleic-acid sequences by determining a mean or median positive insert size of reads covering a genomic coordinate. Such positive inserts can include an area of a DNA or RNA fragment that is covered by neither of two sequencing reads. In contrast to positive-insert-size metrics, in some cases, the genome-classification system 106 determines negative-insert-size metrics for sample nucleic-acid sequences. For instance, the genome-classification system 106 determines a mean or median negative insert size of sequencing reads covering a genomic coordinate—as the negative-insert-size metrics. Such negative inserts can include an overlap between two sequencing reads.

In addition or in the alternative to alignment metrics, the genome-classification system 106 can determine depth metrics that quantify depth of nucleobase calls at genomic coordinates for sample nucleic-acid sequences. A depth metric can, for instance, quantify a number of nucleobase calls that have been determined and aligned at a genomic coordinate. In certain implementations, the genome-classification system 106 determines forward-reverse-depth metrics for sample nucleic-acid sequences by determining a depth on both forward and reverse strands at a genomic coordinate. Additionally or alternatively, the genome-classification system 106 determines normalized-depth metrics for sample nucleic-acid sequences by, for instance, determining depth on a normalized scale at a genomic coordinate. In some such cases, the genome-classification system 106 uses a scale in which a normalized depth of 1 refers to diploid and a normalized depth of 0.5 refers to haploid.

In addition to forward-reverse-depth metrics or normalized-depth metrics, in some cases, the genome-classification system 106 determines depth-under metrics or depth-over metrics for sample nucleic-acid sequences. For example, the genome-classification system 106 can determine a depth-under metric by quantifying a number of nucleobase calls below an expected or threshold depth coverage at a genomic coordinate or genomic region. In some cases, the genome-classification system 106 multiplies a mean depth coverage at a genomic coordinate by −1, adds 1, and sets a minimum value of 0. If a genomic coordinate has a mean depth coverage of 0.75, for instance, the genome-classification system 106 would determine a depth-under metric of 0.25 for the genomic coordinate. By contrast, the genome-classification system 106 can determine a depth-over metric by quantifying a number of nucleobase calls above an expected or threshold depth coverage at a genomic coordinate or genomic region.

As noted above, in some implementations, the genome-classification system 106 determines a peak-count metric by, for instance, determining a distribution of depth for a genomic coordinate or region across genome samples (e.g., a diverse cohort of genome samples) and identifying local maxima for depth coverage from the distribution. In certain implementations, the genome-classification system 106 uses a Gaussian kernel to smooth over depth metrics for a genomic region into a distribution of depth coverage and applies a find-peaks function from a signal processing sub package at SciPy.org to the distribution identify local maxima for depth coverage.

Independent of depth metrics, the genome-classification system 106 can determine call-data-quality metrics that quantify nucleobase-call quality for sample nucleic-acid sequences at genomic coordinates. In certain embodiments, for instance, the genome-classification system 106 determines nucleobase-call-quality metrics by determining a percentage or subset of nucleobase calls satisfying a threshold quality score (e.g., Q20) at a genomic coordinate of an example nucleic-acid sequence (e.g., a reference genome or a nucleic-acid sequence of an ancestral haplotype). To illustrate, the quality score (or Q score) may indicate that a probability of an incorrect nucleobase call at a genomic coordinate is equal to 1 in 100 for a Q20 score, 1 in 1,000 for a Q30 score, 1 in 10,000 for a Q40 score, etc.

In addition or in the alternative to nucleobase-call-quality metrics, in some embodiments, the genome-classification system 106 determines callability metrics for sample nucleic-acid sequences by, for instance, determining a score indicating a correct nucleotide-variant call or nucleobase call at a genomic coordinate. In some cases, the callability metric represents a fraction or percentage of non-N reference positions with a passing genotype call, as implemented by Illumina, Inc. Further, in some implementations, the genome-classification system 106 uses a version of Genome Analysis Toolkit (GATK) to determine callability metrics.

Beyond nucleobase-call-quality metrics or callability metrics, in some embodiments, the genome-classification system 106 determines somatic-quality metrics for sample nucleic-acid sequences by, for instance, determining a score estimating a probability of determining a number of anomalous reads in a tumor sample. For example, a somatic-quality metric can represent an estimate of a probability of determining a given (or more extreme) number of anomalous reads in a tumor sample using a Fisher Exact Test—given counts of anomalous and normal reads in tumor and normal BAM files. In some cases, the genome-classification system 106 using a Phred algorithm to determine a somatic-quality metric and expresses the somatic-quality metric as a Phred-scaled score, such as a quality score (or Q score), that ranges from 0 to 60. Such a quality score may be equal to −10 log10(Probability variant is somatic).

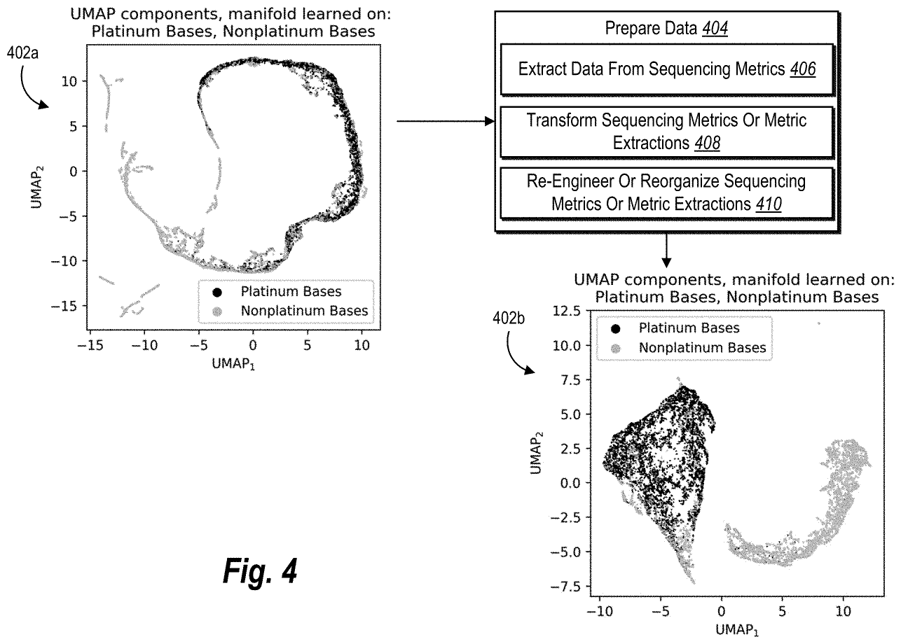

As suggested above, after determining sequencing metrics, the genome-classification system 106 can prepare data from the sequencing metrics for input into a genome-location-classification model. In accordance with one or more embodiments, FIG. 4 illustrates the genome-classification system 106 preparing data 404 from sequencing metrics by (i) extracting data from sequencing metrics 406, (ii) transforming sequencing metrics or metric extractions 408, and (iii) re-engineering or reorganizing sequencing metrics or metric extractions 410. As illustrated by Uniform Manifold Approximation and Projection (UMAP) graphs 402a and 402b and explained further below, the data preparation effectively curates the data for a genome-location-classification model, as measured by the platinum bases and non-platinum bases from regions catalogued by Platinum Genomes. As used herein, the term “platinum base” or “truthset base” represents a nucleobase from a defined confidence region of the Platinum Genomes developed by Illumina, Inc. In particular, a platinum base (or a truthset base) represents a nucleobase from a genomic coordinate with one or both of a defined Mendelian-inheritance pattern and consistent homozygous inheritance.

As depicted by FIG. 4, for instance, the genome-classification system 106 extracts data from sequencing metrics 406 to prepare the data for input into a genome-location-classification model. By extracting data or features from the sequencing metrics, the genome-classification system 106 can summarize information from the sequencing metrics that a genome-location-classification model may not otherwise identify or learn. For instance, in some embodiments, the genome-classification system 106 extracts data from sequencing metrics by determining one or more of (i) a rolling mean of certain sequencing metrics to provide a local summary of sequencing metrics for a genomic coordinate, (ii) a masked rolling mean of certain sequencing metrics to provide a local summary of sequencing metrics without a genomic coordinate, or (iii) statistical measurements from statistical tests that assess a specific hypothesis for a given sequencing metric.

As just mentioned, the genome-classification system 106 can perform various statistical tests to extract data from certain sequencing metrics for input into a genome-location-classification model. In some cases, for instance, the genome-classification system 106 performs a Kolmogorov-Smirnov (KS) test on depth metrics (e.g., forward-reverse-depth metrics, normalized-depth metrics) to determine whether depth is normally distributed across the population of samples. In some cases, the KS test quantifies distances among the depths of sample nucleic-acid sequences from each sample according to an empirical distribution function. As a further example of a statistical test, in certain embodiments, the genome-classification system 106 performs a binomial test on depth metrics (e.g., forward-reverse-depth metrics) to determine whether depth is equally distributed on forward and reverse strands. In certain circumstances, the binomial test determines statistical significance of deviations from an expected distribution of depth into a category for forward strands and reverse strands.

In addition (or in the alternative) to KS tests or binomial tests as statistical tests, the genome-classification system 106 performs a binomial proportion test on call-data-quality metrics (e.g., nucleobase-call-quality metrics) and/or other sequencing metrics to determine whether reads on forward and reverse strands have the same percentage of quality scores satisfying a quality-score threshold (e.g., Q20 score). In some cases, the binomial test determines a binomial distribution of the probability that reads on forward and reverse strands have the same percentage of at least Q20 scores. By contrast, in certain implementations, the genome-classification system 106 performs a Bates distribution test to determine whether the average starting position for a genomic coordinate from a reference genome is halfway through a read for the sample nucleic-acid sequences. For instance, the Bates distribution test can determine a probability distribution of a mean number of the average starting position is halfway through a read.

In addition to extracting data from sequencing metrics, as further shown in FIG. 4, the genome-classification system 106 transforms sequencing metrics or metric extractions 408 to prepare for the data for input into a genome-location-classification model. By transforming the sequencing metrics (or extracted data from the sequencing metrics) into new forms or scales, the genome-classification system 106 can rescale certain sequencing metrics to avoid over training or unnecessarily training the genome-location-classification model. For instance, in some embodiments, the genome-classification system 106 transforms sequencing metrics (or extracted data from the sequencing metrics) by one or more of (i) normalizing sequencing metrics that include counts or total numbers to divide such counts or total numbers by coverage, (ii) standardizing all or some of the sequencing metrics and/or extracted data from the sequencing metrics to be on a same scale, (iii) determining a mean or local mean for sequencing metrics, or (iv) determining, for a sequencing metric, a portion or fraction of reads on the forward strand versus the reverse strand of an original oligonucleotide from a genome sample. By contrast, the genome-classification system 106 optionally does not transform certain sequencing metrics, such as by not transforming mapping-quality metrics, read-position metrics, deletion-size metrics, depth metrics, depth-under metrics, depth-over metrics, positive-insert-size metrics, negative-insert-size metrics, and nucleobase-call-quality metrics.

To illustrate specific transformations, in some embodiments, the genome-classification system 106 coverage normalizes soft-clipping metrics by converting a total number of soft-clipped nucleobases spanning a genomic coordinate into a percentage based on total number of reads from a sample. As a further transformation example, in certain cases, the genome-classification system 106 standardizes depth metrics to become values within a standard deviation, such as with a mean of 0 and a standard deviation of 1. Further, the genome-classification system 106 sometimes determines a local mean for read-reference-mismatch metrics by determining a mean number of nucleobases that do not match a nucleobase of a reference genome at a genomic coordinate or genomic region. As another transformation example, in some implementations, the genome-classification system 106 determines, for a nucleobase-call-quality metric or a depth metric, a portion or fraction of reads on the forward strand versus the reverse strand of an original oligonucleotide from a genome sample. By determining a fraction of forward strand to reverse strand for a sequencing metric, the genome-classification system 106 can generate a forward-fraction metric, such as a forward-fraction-nucleobase-call-quality metric or a forward-fraction-depth metric.

After extracting data from and transforming sequencing metrics, in some embodiments, the genome-classification system 106 re-engineer or reorganize sequencing metrics or metric extractions 410 to prepare the data for input into a genome-location-classification model. By re-engineering or reorganizing certain sequencing metrics or metric extractions, the genome-classification system 106 can package certain sequencing metrics or metric extractions into a format that the genome-location-classification model can process. For instance, the genome-classification system 106 can re-engineer or reorganize sequencing metrics or metric extractions by (i) applying a linear-scaling function to scale certain sequencing metrics or metric extractions; (ii) clipping probability values (p-values) from certain sequencing metrics; (iii) determining an absolute value of certain sequencing metrics or metric extractions; (iv) discretizing certain sequencing metrics to change such metrics from continuous values into categories of values; (v) replacing certain sequencing metrics or metric extractions with other values (e.g., to avoid zero values); or (vi) smooth clipping certain sequencing metrics to minimize outlier effects by log transforming values outside a defined range. By contrast, the genome-classification system 106 optionally does not re-engineer or reorganize certain sequencing metrics, such as mapping-quality metrics, soft-clipping metrics, nucleobase-call-quality metrics, deletion-entropy metrics, depth metrics, read-reference-mismatch metrics, and peak-count metrics.

To illustrate specific re-engineering or reorganizing sequencing metrics, in some embodiments, the genome-classification system 106 applies a linear-scaling function to scale certain sequencing metrics or metric extractions by, for instance, using a linear function of y=(a*x)+b to scale values, where “x” represents an original value for a sequencing metric or a metric extraction, “y” represents a scaled value for the sequencing metric or the metric extraction, and “a” and “b” represent different variables for scaling. In certain cases, the genome-classification system 106 applies a linear-scaling function to values for read-position metrics, depth-under metrics, depth-over metrics, and forward-fraction metrics. As a further example of re-engineering or reorganizing a sequencing metric, in some cases, the genome-classification system 106 replaces a 0.0 value with a 0.5 value for read-position metrics and forward-fraction metrics and/or replaces a 0.0 value with a 1.0e-100 for a binomial proportion test on nucleobase-call-quality metrics. Further, the genome-classification system 106 sometimes determines an absolute value for read-position metrics and forward-fraction metrics.

In addition (or in the alternative) to replacing values or determining absolute values for re-engineering or reorganizing certain sequencing metrics, in some embodiments, the genome-classification system 106 logarithmically smooth clips deletion-size metrics, depth metrics, and depth-over metrics to effectively create deletion-size-clip metrics, depth-clip metrics, and depth-over-clip metrics. For instance, the genome-classification system 106 logarithmically smooth clips deletion-size metrics, normalized depth metrics, and depth-over metrics above a value of 5 while not modifying other values for these sequencing metrics. For a value of 1.5, for instance, the genome-classification system 106 would not modify the value and keep the original value for the corresponding sequencing metric input into a genome-location-classification model. But for a value of 9, the genome-classification system 106 transforms the 9 value using a logarithmic formula of 5+log(9−5+1) to output and use a value of ˜5.7.

Beyond or in place of smooth clipping, in certain cases, the genome-classification system 106 clips p-values from KS tests on depth metrics, binomial tests on depth metrics, binomial proportion test on call-data-quality metrics, or Bates distribution test on read-position metrics. For each value in such statistical tests, for instance, the genome-classification system 106 log-smooths a Phred-scaled p-value above 5.0 to avoid overtraining a genome-location-classification model. For instance, the genome-classification system 106 would log-smooth a Phred-scaled p-value of 40 to become ˜6.5.

To further illustrate specific re-engineering or reorganization of sequencing metrics, in some embodiments, the genome-classification system 106 discretizes continuous values from positive-insert-size metrics and negative-insert-size metrics into categories of values. For instance, the genome-classification system 106 discretizes positive insertions or negative insertions of varying sizes into three categories: insertions below 200 nucleobases in a first category, insertions between 200 and 800 nucleobases in a second category, and insertions above 800 nucleobases in a third category.

As explained further below, in some embodiments, the genome-classification system 106 inputs data extracted, transformed, and rescaled from sequencing metrics into a genome-location-classification model for training or application. For instance, the genome-classification system 106 aggregates the rescaled data from the sequencing metrics for each genomic coordinate and iteratively inputs the rescaled sequencing metric data into the genome-location-classification model along with a genomic-coordinate identifier.

By preparing the data from sequencing metrics as indicated above, the genome-classification system 106 effectively transforms sequencing metrics (or derivations from the sequencing metrics) to indicate the relatively higher or lower reliability of genomic coordinates to a genome-location-classification model. To orthogonally test the effectiveness of such data preparation, researchers executed a UMAP algorithm to (i) visualize nucleobases at particular genomic coordinates according to the sequencing metrics before data preparation in the UMAP graph 402a and (ii) visualize nucleobases at particular genomic coordinates according to the sequencing metrics after data preparation in the UMAP graph 402b, as illustrated in FIG. 4. As the UMAP graphs 402a and 402b indicate, the data preparation effectively separates nucleobase calls from genomic regions with verified variant calls (here, at platinum bases) according to Platinum Genomes and nucleobase calls from genomic regions without verified variant calls (here, at nonplatinum bases) according to Platinum Genomes. Note that the UMAP graphs 402a and 402b do not represent a component of a genome-location-classification model or a component of data preparation, but merely visualize an orthogonal test of the data preparation.

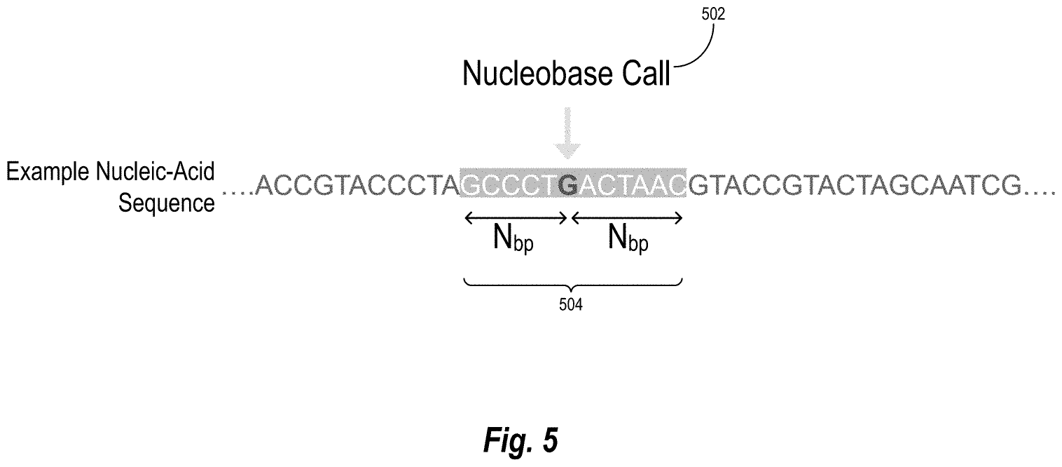

In addition or in the alternative to determining sequencing metrics, in some embodiments, the genome-classification system 106 determines a contextual nucleic-acid subsequence from an example nucleic-acid sequence (e.g., a reference genome, ancestral haplotype) that surrounds a nucleobase call as an input for a genome-location-classification model. In accordance with one or more embodiments, FIG. 5 illustrates an example of the genome-classification system 106 determining a contextual nucleic-acid subsequence 504 corresponding to a nucleobase call 502 as such an input.

As shown in FIG. 5, the genome-classification system 106 identifies the nucleobase call 502 for a particular genomic coordinate. In some cases, the genome-classification system 106 identifies a nucleotide-call variant or nucleotide-call invariant from a VCF file at the genomic coordinate. Based on the genomic coordinate, the genome-classification system 106 further identifies a series of nucleobases from a reference genome that are located both upstream and downstream from the genomic coordinate of the nucleobase call 502 and within a threshold number of genomic coordinates from the genomic coordinate of the nucleobase call 502. As depicted in FIG. 5, the genome-classification system 106 identifies this series of upstream-and-downstream nucleobases from the example nucleic-acid sequence as the contextual nucleic-acid subsequence 504 for the nucleobase call 502. After identification, in some embodiments, the genome-classification system 106 further prepares the contextual nucleic-acid subsequence 504 by applying a vector algorithm (e.g., Nucl2Vec, one-hot vector) to encode the contextual nucleic-acid subsequence 504 into a vector for input into a genome-location-classification model.

When identifying a contextual nucleic-acid subsequence from the example nucleic-acid sequence, the genome-classification system 106 can use a variety of threshold numbers of genomic coordinates. For instance, a contextual nucleic-acid subsequence can include the nucleobases of a reference genome within ten, fifty, one hundred, four hundred, or any other number of genomic coordinates from the genomic coordinate of a particular nucleobase call. As described further below, in some cases, the genome-classification system 106 increases the accuracy with which a genome-location-classification model determines confidence classifications for genomic coordinates as the threshold number of genomic coordinates for nucleobases increases for a contextual nucleic-acid subsequence.

In addition to the threshold number of genomic coordinates varying, in some embodiments, the genome-classification system 106 uses a variety of different variant call types as the nucleobase call from which the threshold number of genomic coordinates is determined. As depicted by FIG. 5, for instance, the genome-classification system 106 identifies an SNV for the nucleobase call 502. In some embodiments, however, the genome-classification system 106 identifies a genomic coordinate (or genomic coordinates) for an indel, structural variation, or CNV as a reference point from which to determine nucleobases within a threshold number of genomic coordinates that make up a contextual nucleic-acid subsequence.

To identify nucleotide-variant calls as a basis for determining contextual nucleic-acid subsequences, in some cases, the genome-classification system 106 uses variant calls from VCF files. To take but one example, the genome-classification system 106 can identify variant calls from the concordance data of a VCF file for NA12878 (or other samples) from the HapMap Project. In one such case, the genome-classification system 106 determines variant calls from 96 replicates of NA12878 as the basis for determining contextual nucleic-acid subsequences for input into a genome-location-classification model and training.

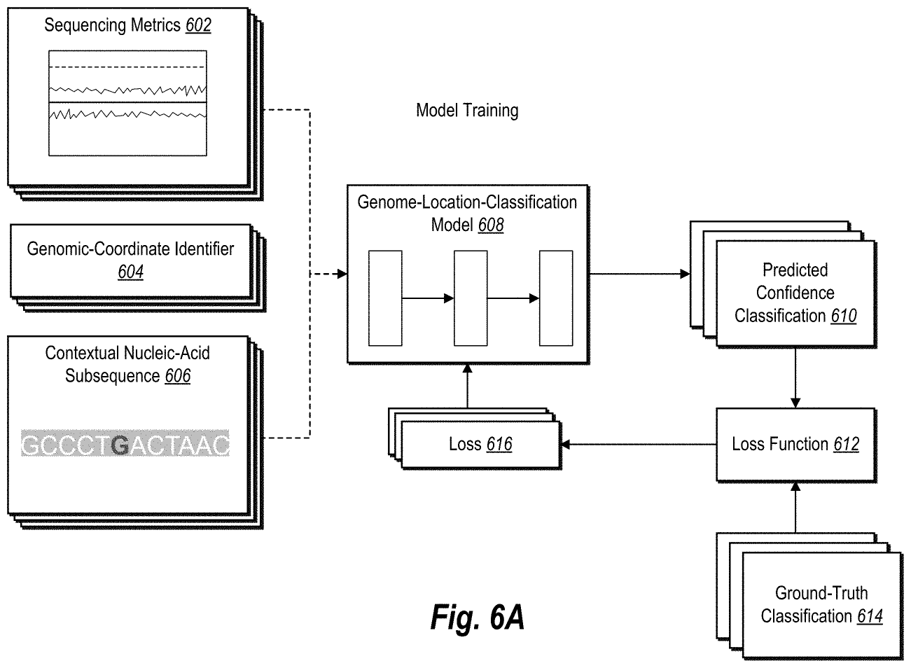

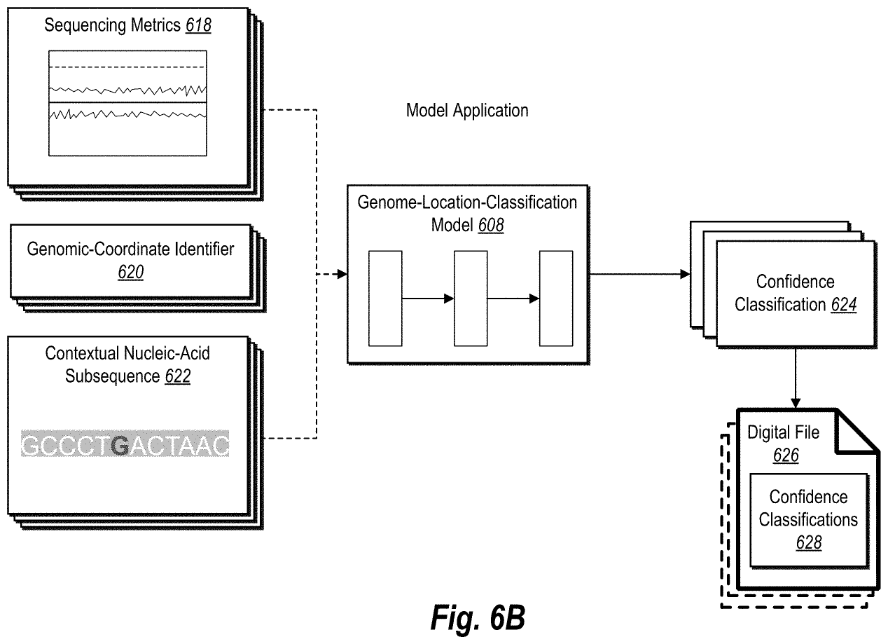

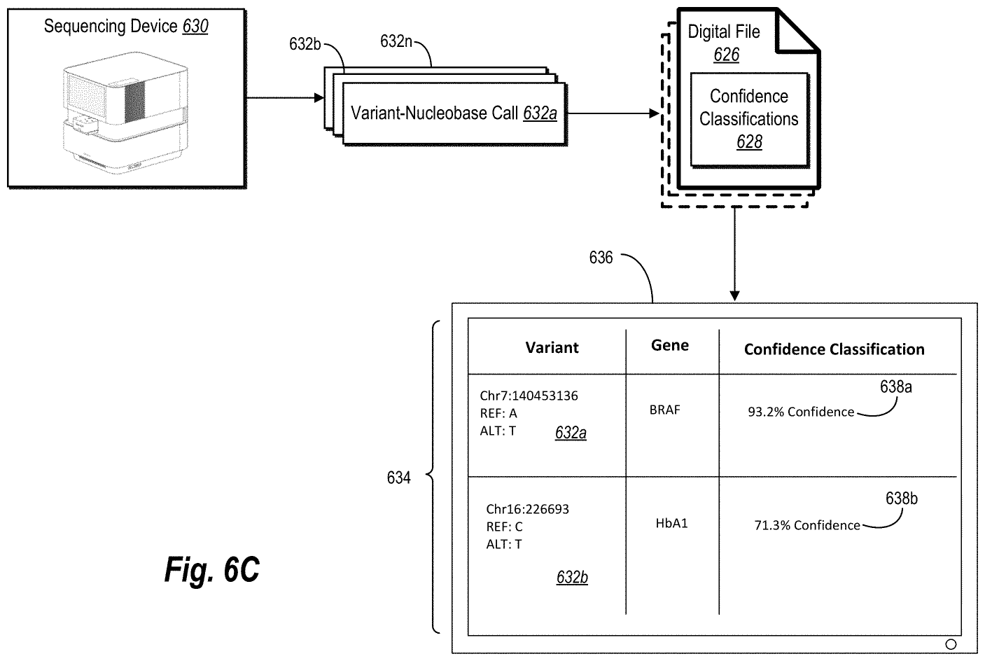

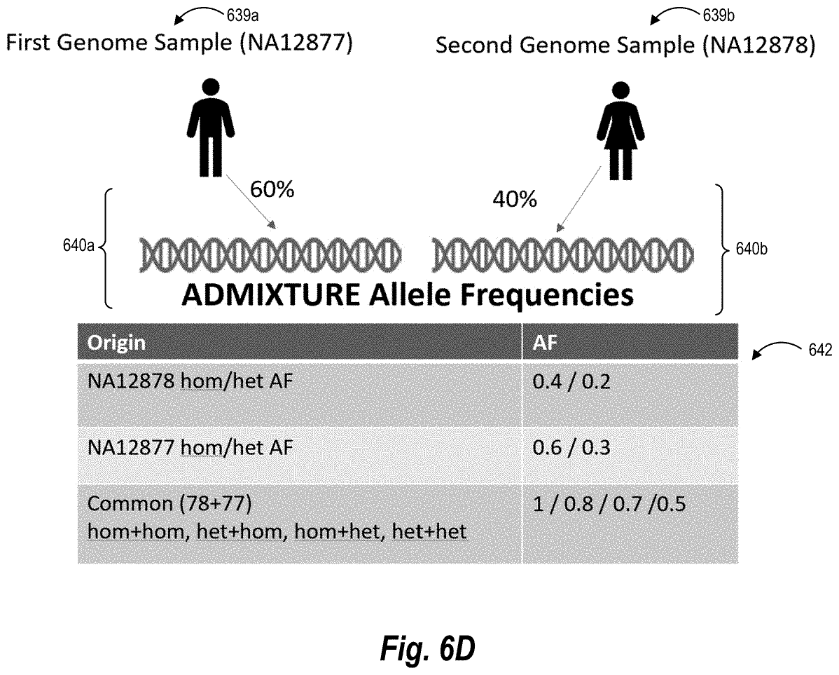

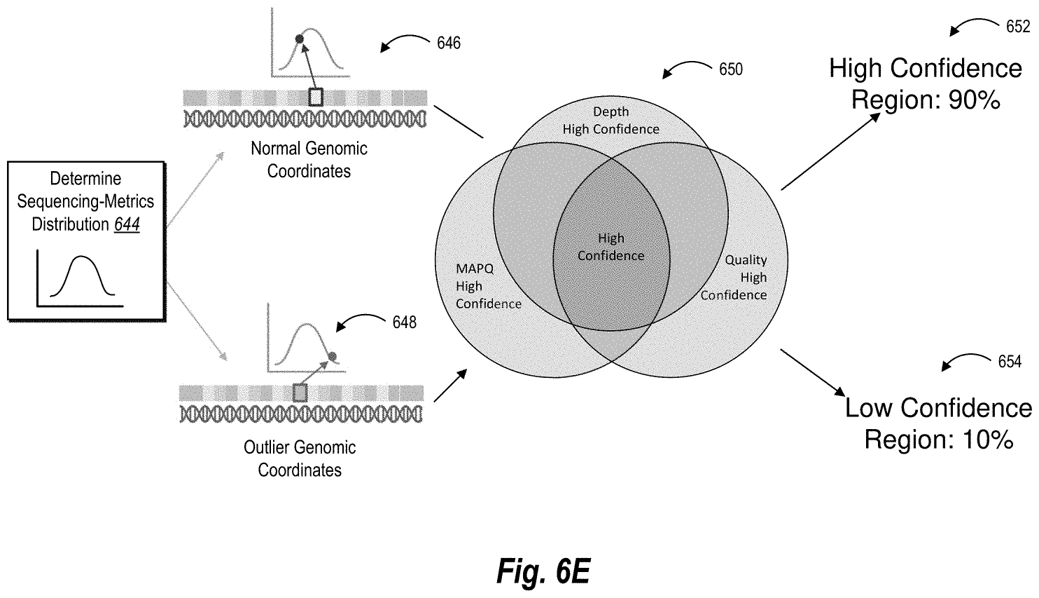

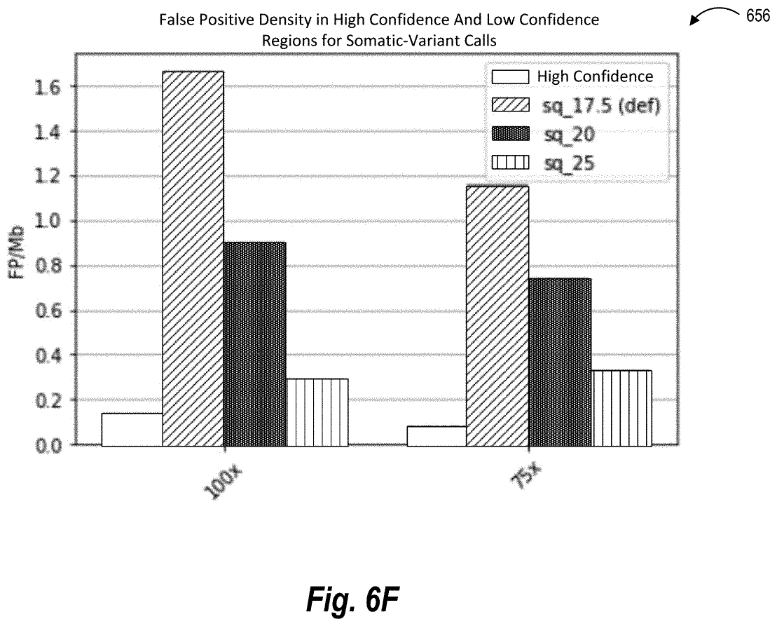

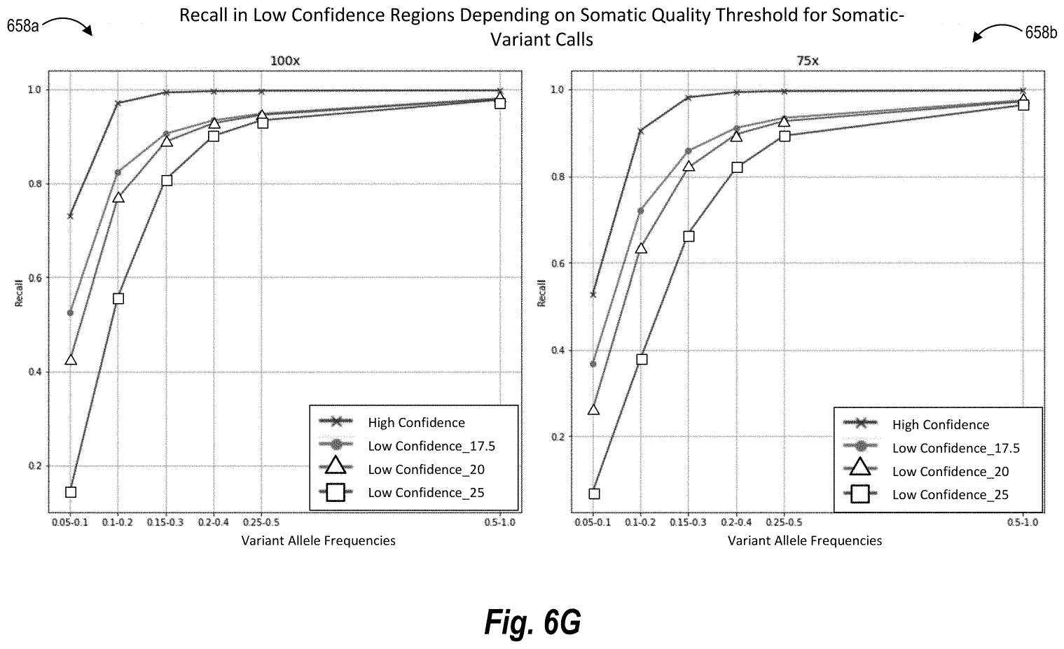

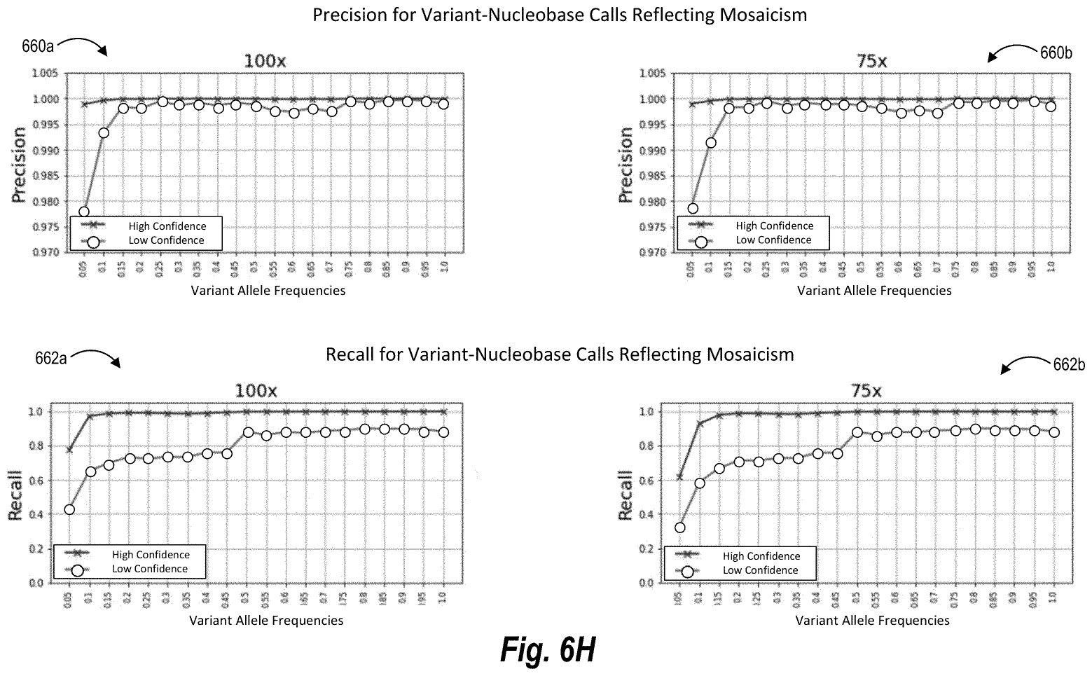

After determining sequencing metrics and contextual nucleic-acid subsequences and preparing the data for input, the genome-classification system 106 trains and applies a genome-location-classification model. In accordance with one or more embodiments, FIGS. 6A-6C illustrate the genome-classification system 106 training and applying a genome-location-classification model 608 to determine confidence classifications for genomic coordinates (or regions) and subsequently providing a confidence indicator for a confidence classification corresponding to a nucleobase call for display on a computing device. As depicted in FIG. 6A, the genome-classification system 106 performs multiple training iterations in which the genome-classification system 106 (i) determines predicted confidence classifications based on one or both of sequencing metrics and contextual nucleic-acid subsequences and (ii) compares such predicted confidence classifications to ground-truth classifications. After training, as shown in FIG. 6B, the genome-classification system 106 applies a trained version of the genome-location-classification model 608 to determine a set of confidence classifications for a set of genomic coordinates (or regions) and generate a digital file comprising the set of confidence classifications. Based on the generated digital file, as shown in FIG. 6C, the genome-classification system 106 provides a confidence classification for a genomic coordinate (or region) of a nucleobase call for display on a graphical user interface.

For simplicity, this disclosure describes an initial training iteration followed by a summary of subsequent training iterations depicted in FIG. 6A. In an initial training iteration depicted by FIG. 6A, for example, the genome-classification system 106 inputs into the genome-location-classification model 608 data derived or prepared from one or both of sequencing metrics 602 and a contextual nucleic-acid subsequence 606 corresponding to a genomic-coordinate identifier 604 for a particular genomic coordinate.

As just suggested and depicted in FIG. 6A, in some embodiments, the genome-classification system 106 inputs data prepared from the sequencing metrics 602 specific to the genomic coordinate for the genomic-coordinate identifier 604—without a corresponding contextual nucleic-acid subsequence for the genomic coordinate. In some such embodiments, the input includes data from one or more of a KS test, a binomial test, a binomial proportion test, or a bates distribution test. By contrast, in certain implementations, the genome-classification system 106 inputs the contextual nucleic-acid subsequence 606 specific to the genomic coordinate for the genomic-coordinate identifier 604—without corresponding sequencing metrics. Alternatively, the genome-classification system 106 inputs data derived or prepared from both of sequencing metrics 602 and the contextual nucleic-acid subsequence 606.

As suggested above, the genome-classification system 106 inputs such data into the genome-location-classification model 608 in a variety of formats. For instance, in some embodiments, the genome-classification system 106 aggregates rescaled data from the sequencing metrics 602 for a genomic coordinate into a vector or matrix comprising each rescaled sequencing metric for the genomic-coordinate identifier 604. In some cases, the genome-classification system 106 aggregates rescaled data from the sequencing metrics 602 for the genomic coordinate corresponding to the genomic-coordinate identifier 604 together with the contextual nucleic-acid subsequence 606 into an input vector or matrix. By contrast, in certain implementations, the genome-classification system 106 aggregates rescaled data from the sequencing metrics 602 for a genomic coordinate corresponding to the genomic-coordinate identifier 604—and rescaled sequencing metrics for each genomic coordinate for the nucleobases in the contextual nucleic-acid subsequence 606—together with the contextual nucleic-acid subsequence 606 into an input vector or matrix.

To illustrate, in some embodiments, the genome-classification system 106 inputs data derived or prepared from the sequencing metrics 602 as a set of numeric arrays into the genome-location-classification model 608. For example, the genome-classification system 106 stores data derived or prepared from the sequencing metrics 602 in a Hierarchical Data Format 5 (HDF5) file and inputs the data as sets of numeric arrays (e.g., single-dimension Python NumPy arrays) into the genome-location-classification model 608.

To further illustrate, in certain implementations, the genome-classification system 106 inputs (into the genome-location-classification model 608) the data derived or prepared from both the sequencing metrics 602 and the contextual nucleic-acid subsequence 606 as a matrix—with a first dimension for a size or length of the contextual nucleic-acid subsequence 606 and a second dimension for the number of individual sequencing metrics and/or derivations from the individual sequencing metrics. For example, the first dimension for a size or length of the contextual nucleic-acid subsequence 606 can include the number of nucleobases in the contextual nucleic-acid subsequence 606 plus one (e.g., 51 dimensions for 25 bases on each side of a nucleobase call, 101 dimensions for 50 bases on each side of a nucleobase call). By contrast, the second dimension for the number of the individual sequencing metrics can include a number of dimensions representing each of individual sequencing metrics, derivations from sequencing metrics, and a vectorized representation of the contextual nucleic-acid subsequence (e.g., one-hot encoded contextual nucleic-acid subsequence that take up 5 positions).

Further, when inputting multiple examples of contextual nucleic-acid subsequences corresponding to multiple nucleobase calls into the genome-location-classification model 608, in some cases, the genome-classification system 106 inputs a three-dimensional tensor. Such a tensor can include a first dimension representing the number of examples, a second dimension representing a size or length of contextual nucleic-acid subsequences, and a third dimension for the number of individual sequencing metrics and/or derivations from the individual sequencing metrics.

When inputting data derived or prepared form the contextual nucleic-acid subsequence 606 into the genome-location-classification model 608, in some cases, the genome-classification system 106 inputs data derived from a single strand of DNA or RNA. For instance, the genome-classification system 106 inputs a vectorized form of a contextual nucleic-acid subsequence from a positive-sense strand or a negative-sense strand of an example nucleic-acid sequence (e.g., ancestral haplotype). In some embodiments, the genome-classification system 106 separately inputs a vectorized form of a contextual nucleic-acid subsequence from both a positive-sense strand and a negative-sense strand of a contextual nucleic-acid subsequence—determined from an example nucleic-acid sequence (e.g., ancestral haplotype)—and determines a confidence classification corresponding to each of the positive-sense strand and the negative-sense strand.

After inputting data derived or prepared from one or both of the sequencing metrics 602 and the contextual nucleic-acid subsequence 606, the genome-classification system 106 executes the genome-location-classification model 608. As indicated above, the genome-location-classification model 608 can take various forms. The genome-location-classification model 608 may be, for instance, a statistical machine-learning model or a neural network. In some cases, the genome-location-classification model takes the form of a logistic regression model, a random forest classifier, a CNN, or a Long Short-Term Memory (LSTM) network, to name a few examples.

For example, in some embodiments, the genome-location-classification model 608 takes the form of a CNN comprising 2 convolutional layers and 1 fully connected layer. By contrast, in certain cases, the genome-location-classification model 608 takes the form of a CNN comprising 8, 12, 20 convolutional layers and 1 fully connected layer. Alternatively, the genome-location-classification model 608 takes the form of a modified Inception Network comprising multiple convolutional layers concatenated together in each layer (e.g., conv3, conv5, conv7, conv9) where each convolutional layer is derived from the same prior layer.

Upon receiving the input data for an initial training iteration, as further shown in FIG. 6A, the genome-location-classification model 608 determines a predicted confidence classification 610 for the genomic coordinate corresponding to the genomic-coordinate identifier 604. In some embodiments, for instance, the predicted confidence classification 610 comprises a label indicating a high-confidence classification, an intermediate-confidence classification, or a low-confidence classification that nucleobases can be accurately determined at the genomic coordinate corresponding to the genomic-coordinate identifier 604. By contrast, in certain implementations, the predicted confidence classification 610 comprises a score indicating a probability or a likelihood that nucleobases can be determined with high confidence at the genomic coordinate corresponding to the genomic-coordinate identifier 604. Based on such a probability or likelihood score, in some cases, the genome-classification system 106 determines a high-confidence classification, an intermediate-confidence classification, or a low-confidence classification.

As indicated above, in certain implementations, the genome-classification system 106 determines confidence classifications for genomic coordinates specific to a variant type. When determining the predicted confidence classification 610, therefore, the genome-classification system 106 can determine a predicted variant confidence classifications for a genomic coordinate specific to SNPS, insertions of various sizes (e.g., short insertions, intermediate insertions, or long insertions), deletions of various sizes (e.g., short deletions, intermediate deletions, or long deletions), structural variations of various sizes, or CNVs of various sizes. Additionally or alternatively, the genome-classification system 106 can determine a predicted variant confidence classification for a genomic coordinate specific to a somatic-nucleobase variant or a germline-nucleobase variant, such as a somatic-nucleobase variant reflecting cancer or somatic mosaicism or a germline-nucleobase variant reflecting germline mosaicism. To train the genome-location-classification model 608 to generate variant confidence classifications specific to a variant type, as explained below, the genome-classification system 106 uses ground-truth classifications specific to the corresponding variant type.

As further shown in FIG. 6A, after determining the predicted confidence classification 610, the genome-classification system 106 compares the predicted confidence classification 610 to a ground-truth classification 614 for the genomic coordinate corresponding to the genomic-coordinate identifier 604. For instance, in some implementations, the genome-classification system 106 uses a loss function 612 to compare (and determine any difference) between the predicted confidence classification 610 and the ground-truth classification 614. As explained below, in some cases, the ground-truth classification 614 reflects a Mendelian-inheritance pattern or a replicate concordance of nucleobase calls at the genomic coordinate corresponding to the genomic-coordinate identifier 604. As further shown in FIG. 6A, the genome-classification system 106 determines a loss 616 from the predicted confidence classification 610 and the ground-truth classification 614 utilizing the loss function 612.

Depending on the form of the genome-location-classification model 608, the genome-classification system 106 can use a variety of loss functions for the loss function 612. In certain embodiments, for instance, the genome-classification system 106 uses a logistic loss (e.g., for a logistic regression model), a Gini impurity or an information gain (e.g., for a random forest classifier), or a cross-entropy-loss function or a least-squared-error function (e.g., for a CNN, LSTM).

As indicated above, the genome-classification system 106 can use a variety of bases or grounds for identifying ground-truth classifications. In some embodiments, for instance, the genome-classification system 106 labels a genomic coordinate with a ground-truth classification of high confidence when the genomic coordinate corresponds to a nucleotide-variant call having one (or any combination) of the following characteristics: a Mendelian-inheritance pattern, consistent homozygous inheritance (e.g., a genomic coordinate where the same alleles come from both parents), or a threshold number (or threshold portion) of replicates exhibiting the nucleotide-variant call at the genomic coordinate. For instance, the genome-classification system 106 can label a genomic coordinate with a ground-truth classification of high confidence when the threshold number (or threshold portion) of replicates equals or exceeds 56% of sample nucleic-acid sequences (e.g., 54 of 96 samples) exhibiting a nucleotide-variant call. In one additional example embodiment, the genome-classification system 106 labels a genomic coordinate with a ground-truth classification of high confidence when the genomic coordinate corresponds to a platinum base or truthset base from the Platinum Genomes and of a low confidence of low confidence when the genomic coordinate does not correspond to a platinum base or truthset base from the Platinum Genomes.

By contrast, in some cases, the genome-classification system 106 labels a genomic coordinate with a ground-truth classification of low confidence when the genomic coordinate corresponds to a nucleotide-variant call having one (or any combination) of the following characteristics: a non-Mendelian-inheritance pattern, failing or inconsistent homozygous inheritance, or a threshold number (or threshold portion) of replicates exhibiting the nucleotide-variant call at the genomic coordinate. For instance, the genome-classification system 106 can label a genomic coordinate with a ground-truth classification of low confidence when the threshold number (or threshold portion) of replicates equals or falls below 15% of sample nucleic-acid sequences (e.g., 14 of 96 samples) exhibiting a nucleotide-variant call.

In some embodiments, the genome-classification system 106 optionally uses a label for intermediate confidence. For instance, the genome-classification system 106 labels a genomic coordinate with a ground-truth classification of intermediate confidence when the genomic coordinate corresponds to a nucleotide-variant call having at most two of a Mendelian-inheritance pattern, consistent homozygous inheritance (e.g., a genomic coordinate part of a gene where the same alleles come from both parents), and reproducibility across technical replicates. But the genome-classification system 106 can also use labels for high-confidence classification and low-confidence classification as ground-truth classifications—without an intermediate-confidence classification.

As indicated above, in some cases, the genome-classification system 106 labels genomic coordinates with a ground-truth classification for a specific type of nucleotide-variant call. For instance, the genome-classification system 106 labels genomic coordinates with a ground-truth classification for one or more of SNPs, insertions of various sizes, deletions of various sizes, structural variations of various sizes, CNVs of various sizes, somatic-nucleobase variants reflecting cancer or somatic mosaicism, or germline-nucleobase variants reflecting germline mosaicism. Such somatic mosaicism can include either or both of mosaicism in cancer cells or healthy cells with mosaic variations. In certain implementations, the genome-classification system 106 labels genomic coordinates with a ground-truth classification specific to a type of nucleotide-variant call based on a threshold number (or threshold portion) of replicates exhibiting the nucleotide-variant call at the genomic coordinate.

As shown in Table 1 below, researchers identified a threshold replicate count for identifying specific types of nucleotide-variant calls (e.g., SNPs, deletions, insertions) at a genomic coordinate as bases for labeling the genomic coordinate with a ground-truth classification of high confidence or low confidence. In particular, the researchers determined a positive predictive value (PPV) for rates of detecting a stochastic false positive of a specific type of nucleotide-variant call based on a technical replicate count of the specific type of nucleotide-variant call from 96 total samples at a given genomic coordinate. By comparing the replicate count to PPV, the researchers determined a minimum replicate count reported in Table 1 at which a rate of stochastic false positive for the nucleotide-variant call satisfies a target threshold, such as a target threshold of less than 0.05% rate of stochastic false positive nucleotide-variant calls at a genomic coordinate for a ground-truth classification of high confidence.

TABLE 1

| | | Max | | Min | | |
|---|---|---|---|---|---|---|
| | | count | Low | count | High | |
| | | for low | confi- | for high | confi- | |
| | | confi- | dence | confi- | dence | Mean high |
| Variant | Size | dence | site | dence | site | confidence |
| type | range | set | count* | set | count | reproducibility |
| SNPs | NA | 1 | 860,100 | 54 | 4,059,704 | 95.07% |
| Deletions | 1-5 | 1 | 37,278 | 64 | 246,153 | 95.22% |
| Deletions | 5-15 | 1 | 3,994 | 63 | 33,788 | 93.83% |
| Deletions | 15+ | 1 | 5,205 | 70 | 16,228 | 94.14% |
| Insertions | 1-5 | 1 | 29,895 | 63 | 170,639 | 95.25% |
| Insertions | 5-15 | 1 | 5,480 | 80 | 8,990 | 97.39% |
| Insertions | 15+ | 1 | 4,789 | 47 | 5,542 | 81.92% |

As reported in Table 1, short deletions span 1-5 nucleobases, intermediate deletions span 5-15 nucleobases, long deletions span more than 15 nucleobases and can include (or be shorter than) deletions of 50 nucleobases, short insertions span 1-5 nucleobases, intermediate insertions span 5-15 nucleobases, and long insertions span more than 15 nucleobases and can include (or be shorter than) insertions of 50 nucleobases. Researchers determined a minimum replicate count of 54, 64, 63, 70, 63, 80, and 47 out of a total 96 samples as thresholds for labeling a genomic coordinate with a ground-truth classification of high confidence for SNPs, short deletions, intermediate deletions, long deletions, short insertions, intermediate insertions, and long insertions, respectively. As shown in Table 1, the minimum replicate counts for labeling genomic coordinates with a ground-truth classification of high confidence—above the corresponding minimum replicate count just listed—correspond to a mean confidence of 95.07%, 95.22%, 93.83%, 94.14%, 95.25%, 97.39%, and 81.92% of variant-call reproducibility for SNPs, short deletions, intermediate deletions, long deletions, short insertions, intermediate insertions, and long insertions, respectively. In other words, the mean high confidence reproducibility in Table 1 indicate the minimum number of replications of a variant to set a threshold for high confidence. Table 1 further reports a number of sites (e.g., genomic coordinates or genomic regions) that the genome-classification system 106 labels with ground-truth classifications of high confidence or low confidence for SNPs, deletions, and insertions in accordance with one or more embodiments.

In the alternative to labels, in some embodiments, the genome-classification system 106 assigns genomic coordinates with a ground-truth classification reflecting a confidence score with weights for whether the genomic coordinate corresponds to a nucleotide-variant call having one or more of a Mendelian-inheritance pattern, a consistent homozygous inheritance, or reproducibility across technical replicates. For instance, in some embodiments, such a confidence score for a genomic coordinate represents the sum or product of one value point for Mendelian-inheritance pattern multiplied by a first weight, one value point for consistent homozygous inheritance multiplied by a second weight, and one value point for reproducibility across technical replicates multiplied by a third weight.

Based on the determined loss 616 from the loss function 612, the genome-classification system 106 subsequently adjusts parameters of the genome-location-classification model 608. By adjusting the parameters, the genome-classification system 106 increases the accuracy with which the genome-location-classification model 608 accurately determines predicted confidence classifications over training iterations. After the initial training iteration and parameter adjustment, as shown by FIG. 6A, the genome-classification system 106 further determines predicted confidence classifications for different genomic coordinates based on data derived or prepared from one or both of sequencing metrics and contextual nucleic-acid subsequences for the different genomic coordinates. In some cases, the genome-classification system 106 performs training iterations until the parameters (e.g., value or weights) of the genome-location-classification model 608 do not change significantly across training iterations or otherwise satisfy a convergence criteria.

Although FIG. 6A depicts training iterations that generate predicted confidence classifications for genomic coordinates, in some embodiments, the genome-classification system 106 likewise inputs data and determines confidence classifications for genomic regions. In training iterations of such embodiments, the genome-classification system 106 inputs a genomic-region identifier for a genomic region and data derived or prepared from one or both of sequencing metrics and contextual nucleic-acid subsequences for each genomic coordinate within the genomic region. The genome-classification system 106 further uses the genome-location-classification model 608 to determine a predicted confidence classification for the genomic region based on such genomic-region-specific inputs. The genome-classification system 106 likewise uses a loss function to compare the predicted confidence classifications for the genomic region and a ground-truth classification for the genomic region and adjusts parameters of the genome-location-classification model 608 based on a determined loss from the loss function.

After training the genome-location-classification model 608, and as depicted in FIG. 6B, the genome-classification system 106 applies a trained version of the genome-location-classification model 608 to determine a set of confidence classifications for a set of genomic coordinates and generate a digital file comprising the set of confidence classifications. Similar to the training process described above, as shown in FIG. 6B, the genome-classification system 106 determines confidence classifications for genomic coordinate after genomic coordinate based on data derived or prepared from one or both of sequencing metrics and contextual nucleic-acid subsequences corresponding to the particular genomic coordinates. For simplicity, this disclosure describes an initial application iteration or initial process to determine a single confidence classification followed by a summary of subsequent application iterations depicted in FIG. 6B.

In an initial application iteration depicted in FIG. 6B, for instance, the genome-classification system 106 inputs into the trained version of the genome-location-classification model 608 data derived or prepared from one or both of sequencing metrics 618 and a contextual nucleic-acid subsequence 622 corresponding to a genomic-coordinate identifier 620 for a particular-genomic coordinate. As when training, the genome-classification system 106 can input any combination of data prepared from the sequencing metrics 618 specific to the genomic coordinate and/or the contextual nucleic-acid subsequence 622 specific to the genomic coordinate corresponding to the genomic-coordinate identifier 620. The genome-classification system 106 can likewise input data prepared from the sequencing metrics 618 and/or the contextual nucleic-acid subsequence 622 by using a same format of input vector or input matrix as described above. The contextual nucleic-acid subsequence 622 input into the trained version of the genome-location-classification model 608 may likewise be a single strand of DNA or RNA (e.g., positive-sense strand or negative sense-strand). In some embodiments, however, the genome-classification system 106 uses a different set of sequencing metrics and/or a different set of contextual nucleic-acid subsequences (and corresponding nucleobase calls) for applying the trained version of the genome-location-classification model 608 than the sequencing metrics and contextual nucleic-acid subsequences used for training.

As further shown in FIG. 6B in an initial application iteration, the trained version of the genome-location-classification model 608 determines a confidence classification 624 for the genomic coordinate corresponding to the genomic-coordinate identifier 620. Consistent with the training above, the confidence classification 624 can comprise (i) a label for a high-confidence classification, an intermediate-confidence classification, or a low-confidence classification that nucleobases can be accurately determined at the genomic coordinate corresponding to the genomic-coordinate identifier 620 or, alternatively, (ii) a score indicating a probability or a likelihood that nucleobases can be determined with high confidence at the genomic coordinate corresponding to the genomic-coordinate identifier 620. Based on the type of ground-truth classifications used for training the genome-location-classification model 608, the confidence classification 624 can likewise be specific to a type of nucleotide-variant call, such as specific to one or more of SNPs, insertions of various sizes, deletions of various sizes, structural variations of various sizes, CNVs of various sizes, somatic-nucleobase variants reflecting cancer or somatic mosaicism, or germline-nucleobase variants reflecting germline mosaicism.

After the initial application iteration, the genome-classification system 106 further determines confidence classifications for different genomic coordinates based on data derived or prepared from one or both of sequencing metrics and contextual nucleic-acid subsequences for the different genomic coordinates. Upon finishing such application iterations, as shown in FIG. 6B, the genome-classification system 106 determines a set of confidence classifications for a set of genomic coordinates based on data derived or prepared from a set of sequencing metrics and contextual nucleic-acid subsequences. In some cases, the set of confidence classifications comprises a confidence classification for each genomic coordinate in a reference genome. By contrast, in certain implementations, the set of confidence classifications comprises a confidence classification for some (but not all) genomic coordinates in a reference genome.

As further shown in FIG. 6B, the genome-classification system 106 further generates a digital file 626 comprising confidence classifications 628. As depicted in FIG. 6B, the confidence classifications 628 comprise the set of confidence classifications for the set of genomic coordinates generated by the genome-location-classification model 608 in FIG. 6B. As with the confidence classification 624—and depending on the type of ground-truth classifications used for training the genome-location-classification model 608—the confidence classifications 628 can likewise be specific to a type of nucleotide-variant call, such as specific to one or more of SNPs, insertions of various size, deletions of various size, structural variations, CNVs, somatic-nucleobase variants reflecting cancer or somatic mosaicism, or germline-nucleobase variants reflecting germline mosaicism.

To generate or modify the digital file 626, in certain implementations, the genome-classification system 106 generates or modifies a BED file to include an annotation for each genomic coordinate comprising a corresponding confidence classification. By contrast, in some embodiments, the genome-classification system 106 generates or modifies a WIG file, BAM file, VCF file, a Microarray file, or other suitable digital file type to include the confidence classifications 628. As further indicated by FIG. 6B, in some embodiments, the genome-classification system 106 can generate separate digital files each comprising different confidence-classification types from the predicted confidence classifications (e.g., a different digital file for each of high-confidence classifications, intermediate-confidence classifications, low-confidence classifications).

Although FIG. 6B depicts application iterations that generate confidence classifications for genomic coordinates, in some embodiments, the genome-classification system 106 likewise inputs data and determines confidence classifications for genomic regions. In application iterations of such embodiments, the genome-classification system 106 inputs a genomic-region identifier for a genomic region and data derived or prepared from one or both of sequencing metrics and contextual nucleic-acid subsequences for each genomic coordinate within the genomic region. The genome-classification system 106 further uses the genome-location-classification model 608 to determine a confidence classification for the genomic region based on such genomic-region-specific inputs.

After generating the digital file 626 (e.g., a part of separate digital files), in some cases, the genome-classification system 106 uses the digital file 626 to provide a specific confidence classification for a genomic coordinate (or region) of a nucleobase call for display on a graphical user interface. In accordance with one or more embodiments, FIG. 6C illustrates the sequencing system 104 or the genome-classification system 106 identifying and displaying particular confidence classifications from the genome-location-classification model 608 corresponding to particular genomic coordinates of nucleotide-variant calls.

As indicated by FIG. 6C, for instance, a sequencing device 630 incorporates nucleobases into a sample nucleic-acid sequence during sequencing and captures corresponding images (or other data) indicating the incorporated nucleobases. Based on the images or other data, the sequencing system 104 or the genome-classification system 106 detect variant-nucleobase calls 632a, 632b, and 632n within the sample nucleic-acid sequence at genomic coordinates. In some embodiments, the variant-nucleobase calls 632a-632n represent SNVs, nucleobase insertions, nucleobase deletions, structural variations, CNVs. Additionally, or alternatively, in certain implementations, the variant-nucleobase calls 632a-632n represent somatic-nucleobase variants reflecting cancer or somatic mosaicism or germline-nucleobase variants reflecting germline mosaicism. The variant-nucleobase calls 632a-632n may likewise be caused by a genetic modification or an epigenetic modification.

As further depicted in FIG. 6C, the genome-classification system 106 integrates the variant-nucleobase calls 632a-632n with one or more of the confidence classifications 628 from the digital file 626 (or from one of multiple digital files). For instance, in some cases, the genome-classification system 106 encodes the variant-nucleobase calls 632a-632n into the digital file 626, compares the variant-nucleobase calls 632a-632n with the confidence classifications 628 from the digital file 626 (or from one of multiple digital files), or retrieves the confidence classifications 628 from the digital file 626 to integrate within a separate digital file for the variant-nucleobase calls 632a-632n (e.g., VCF file). Additionally, or alternatively, in certain implementations, the digital file 626 includes a look-up table for genomic coordinates corresponding to confidence classifications, such as different look-up tables for different variant types in which a genomic coordinate includes a corresponding confidence classification. Regardless of how such integration occurs, the genome-classification system 106 identifies particular confidence classifications from the confidence classifications 628 for the particular genomic coordinates of the variant-nucleobase calls 632a-632n.

In addition to including the variant-nucleobase calls 632a-632n, in some cases, the genome-classification system 106 identifies variant-nucleobase calls or non-variant-nucleobase calls in the digital file 214 suggested for orthogonal validation using a different sequencing method. When variant-nucleobase calls are located at genomic coordinates corresponding to a confidence classification of lower reliability (e.g., low-confidence classification or below a confidence-score threshold) for a particular type of variant, for instance, the genome-classification system 106 includes identifiers for such variant-nucleobase calls in the digital file 214 to suggest orthogonal validation. By using certain confidence classifications as confidence thresholds, the genome-classification system 106 can flag particular variant-nucleobase calls or non-variant-nucleobase calls that a single sequencing pipeline cannot determine with sufficient confidence.

After identifying such confidence classifications from the digital file 626, as further shown in FIG. 6C, the genome-classification system 106 provides to a computing device 636 confidence indicators of particular confidence classifications for genomic coordinates of the variant-nucleobase calls 632a-632n. For example, as depicted in FIG. 6C, the sequencing system 104 or the genome-classification system 106 provides the confidence indicators 638a and 638b of confidence classifications for display within a graphical user interface 634 of the computing device 636—along with genomic coordinates for the variant-nucleobase calls 632a and 632b and identifiers for corresponding genes. By providing the confidence indicators 638a and 638b, the genome-classification system 106 provides clinicians, test subjects, or other people with critical information indicating a reliability of the variant-nucleobase calls 632a and 632b for certain genes.

As suggested above, in some embodiments, the genome-classification system 106 trains or applies a genome-location-classification model to determine confidence classifications specific to somatic-nucleobase variants reflecting cancer or somatic mosaicism or specific to germline-nucleobase variants. To train such a genome-location-classification model, in some embodiments, the genome-classification system 106 determines subsets of nucleic-acid sequences from different genome samples that simulate nucleobase variants from a type of cancer or mosaicism. The genome-classification system 106 further determines certain sequencing metrics for the sample nucleic-acid sequences with respect to genomic coordinates of a reference genome. Based on these sequencing metrics, the genome-classification system 106 generates ground-truth classifications specific to both particular genomic coordinates and particular variant-nucleobase calls, such as somatic-nucleobase variants or germline-nucleobase variants reflecting mosaicism. Using the ground-truth classifications, as described above, the genome-classification system 106 can further train a genome-location-classification model to determine confidence classifications specific to both genomic coordinates and the type of variant-nucleobase calls.

In accordance with one or more embodiments, FIGS. 6D-6H illustrate the genome-classification system 106 determining ground-truth classifications based on one or both of (i) certain sequencing metrics for sample nucleic-acid sequences from genome samples (e.g., a diverse cohort of genome samples as explained above) and (ii) variant-call data for an admixture of genome samples reflecting cancer or mosaicism (e.g., recall or precision rates for calling specific types of variants for an admixture of genome samples reflecting cancer or mosaicism). As depicted in FIG. 6D, the genome-classification system 106 determines subsets (e.g., percentages) of sample nucleic-acid sequences from a combination of male and female genome samples that together simulate variant-allele frequencies of a genome sample with cancer or mosaicism. As shown in FIG. 6E, the genome-classification system 106 determines genomic coordinates exhibiting normal behavior in one or more of depth metrics, mapping-quality metrics, or nucleobase-call-quality metrics for the sample nucleic-acid sequences as a basis for determining ground-truth classifications for high-confidence genomic coordinates. As further depicted in FIGS. 6F-6H, the genome-classification system 106 determines ground-truth classifications based further on one or both of somatic-quality metrics for nucleobase calls from the sample nucleic-acid sequences and recall or precision rates for determining specific type of variant-nucleobase calls based on an admixture of genome samples.

As shown in FIG. 6D, for instance, the genome-classification system 106 determines subsets of sample nucleic-acid sequences from different genome samples forming an admixture genome. When the corresponding sample-nucleic-acid-sequence subsets are mixed together, the admixture genome simulates a genome sample with cancer or mosaicism. To simulate such a genome sample with cancer or mosaicism, for instance, the genome-classification system 106 determines a percentage of sample nucleic-acid sequences 640a from a first genome sample 639a and a percentage of sample nucleic-acid sequences 640b from a second genome sample 639b that, when mixed together, simulate variant-allele frequencies of a genome sample exhibiting characteristics of cancer or mosaicism. As part of determining the subsets of sample nucleic-acid sequences 640a and 640b, the genome-classification system 106 estimates the variant-allele frequencies of different subset mixtures (or percentage mixtures) from truthset bases of Platinum Genomes for the first genome sample 639a and the second genome sample 639b.

According to some embodiments, the genome-classification system 106 uses sample nucleic-acid sequences from an admixture genome—rather than a single, naturally occurring genome—because sequencing systems often cannot consistently or accurately detect nucleobase variants reflecting cancer or mosaicism in sequences from naturally occurring genomes. For instance, a tumor that metastasizes may mutate nucleobases in the DNA of some somatic cell types, but not other somatic cell types. Indeed, some tumors can affect all cells of a particular cell type, such as leukemia spreading in the blood, making a tumor-only sample exclusively available and making it impractical or impossible to obtain a control sample. In different biopsy tissue samples or at different biopsy times, the DNA extracted from a naturally occurring genome with cancer can have significantly different nucleobase allele frequencies—making a sample of a naturally occurring genome an unpredictable sample to estimate variant allele frequencies caused by some cancers. To avoid the unpredictable variability of nucleobase variants in the DNA of cancer or healthy cells, in some implementations, the genome-classification system 106 determines an admixture genome that simulates variants reflecting cancer.

In contrast to cancer-caused variants, naturally occurring mosaicism in the DNA of a sample can exhibit uncommon variants that are difficult to detect during sequencing—regardless of whether the mosaicism is caused by a tumor, genetic inheritance, replication errors, or some other factor. While a single person may have a small percentage of DNA exhibiting mosaicism, many existing sequencing systems cannot detect common nucleobase variants reflecting the mosaicism—unless the sequencing systems sequences oligonucleotides from a much larger group of samples with that type of mosaicism. To create a training genome sample without finding a rare group of samples exhibiting mosaicism, in certain embodiments, the genome-classification system 106 determines an admixture genome to simulate variants reflecting somatic mosaicism or germline mosaicism.

FIG. 6D illustrates an example of the genome-classification system 106 determining subsets of sample nucleic-acid sequences for one such admixture genome and determining corresponding variant allele frequencies. As depicted in FIG. 6D, the genome-classification system 106 determines the variant-allele frequencies for SNPs of both heterozygous and homozygous alleles for an admixture genome. According to the percentages reflected by the subset of sample nucleic-acid sequences 640a (here, 60%) and the subset of sample nucleic-acid sequences 640b (here, 40%), the genome-classification system 106 determines or predicts the relevant variant allele frequencies by referencing the truthset bases of the first genome sample 639a (e.g., NA12877) and the second genome sample 639b (e.g., NA12878) from Platinum Genomes. While FIG. 6D depicts variant allele frequencies for SNPs from an admixture genome, the genome-classification system 106 can determine admixture genomes and variant allele frequencies for other specific variants types, such as insertions, deletions, structural variations, or CNVs.

As shown in an allele-frequency table 642 presented in FIG. 6D, for instance, the genome-classification system 106 determines that unique homozygous alleles and unique heterozygous alleles from the second genome sample 639b occur at variant allele frequencies of 0.4 and 0.2, respectively, in the admixture genome. As further shown, the genome-classification system 106 determines that unique homozygous alleles and unique heterozygous alleles from the first genome sample 639a occur at variant allele frequencies of 0.6 and 0.3, respectively, in the admixture genome. By contrast, the genome-classification system 106 determines that common alleles present in the 60%-and-40% admixture genome as homozygous-homozygous combinations, heterozygous-homozygous combinations, homozygous-heterozygous combinations, and heterozygous-heterozygous combinations—according to the corresponding allele zygosities in the second genome sample 639b and the first genome sample 639a—occur at variant allele frequencies of 1.0, 0.8, 0.7 and 0.5, respectively.

To select a suitable admixture genome representative of a genome sample with cancer or mosaicism, the genome-classification system 106 can determine variant allele frequencies from truthset bases of various combinations (and percentages) of genome samples in a given admixture genome. In addition to the variant allele frequencies present in the 60%-and-40% admixture genome depicted in FIG. 6D, in some embodiments, the genome-classification system 106 determines variant allele frequencies for other possible admixture genomes to simulate a genome sample with cancer or mosaicism. For example, the genome-classification system 106 determines that 30% of sample nucleic-acid sequences from the first genome sample 639a and 70% of sample nucleic-acid sequences from the second genome sample 639b would produce unique homozygous alleles from the first genome sample 639a and from the second genome sample 639b at variant allele frequencies of 0.7 and 0.3, respectively, as well as unique heterozygous alleles from the first genome sample 639a and from the second genome sample 639b at variant allele frequencies of 0.35 and 0.15, respectively. By contrast, the genome-classification system 106 determines or predicts that common alleles present in such a 30%-and-70% admixture genome as homozygous-homozygous combinations, heterozygous-homozygous combinations, homozygous-heterozygous combinations, and heterozygous-heterozygous combinations—according to the same 30% and 70% admixture—would produce variant allele frequencies of 1.0, 0.85, 0.65 and 0.5, respectively.

In addition to determining various admixture genomes from the first genome sample 639a and the second genome sample 639b, in certain implementations, the genome-classification system 106 determines variant allele frequencies from combinations of different sample genomes to identify a suitable admixture genome simulating a genome sample with cancer or mosaicism. By determining variant allele frequencies for a variety of admixture genomes, the genome-classification system 106 can select the admixture genome that more closely (or most closely) simulates the variant allele frequencies of a target type or cancer or mosaicism.

As indicated above, the genome-classification system 106 can generate ground-truth classifications specific to somatic-nucleobase variants reflecting cancer or mosaicism or specific to germline-nucleobase variants based in part on certain sequencing metrics. As shown in FIG. 6E, in some embodiments, the genome-classification system 106 sorts or labels genomic coordinates with a high-confidence classification (or other confidence classification) by (i) determining a sequencing-metrics distribution 644 for sample nucleic-acid sequences from genome samples (e.g., a diverse cohort of genome samples as explained above) across genomic coordinates and (ii) identifying genomic coordinates with certain sequencing metrics that fall within a target part of a normal distribution. In the depicted example, the genome-classification system 106 identifies genomic coordinates within a high-confidence region 652 when they exhibit depth metrics, mapping-quality metrics, and nucleobase-call-quality metrics within a standard deviation of a normal distribution for each of the three sequencing metrics. As discussed below, genomic coordinates that exhibit normal depth metrics, mapping-quality metrics, and nucleobase-call-quality metrics—and are accordingly part of the high-confidence region 652—also exhibit better precision for determining variant-nucleobase calls based on an admixture of genome samples.

As shown in FIG. 6E, the genome-classification system 106 determines the sequencing-metrics distribution 644 for sample nucleic-acid sequences from genome samples (e.g., a diverse cohort of genome samples) at genomic coordinates of a reference genome. To determine such a distribution, the genome-classification system 106 system determines sequencing metrics for sequenced genome samples from a diverse cohort and determines a distribution of the sequencing metrics according to different genomic coordinates. For instance, in certain cases, the genome-classification system 106 determines nucleobases calls for genome samples (e.g., by using a tumor-only analysis in DRAGEN Somatic Pipeline) and determines sequencing metrics for the determined sequence for the genome samples. In some embodiments, the genome-classification system 106 determines depth metrics, mapping-quality metrics, and nucleobase-call-quality metrics for the sample nucleic-acid sequences with respect to each genomic coordinate. By contrast, in certain implementations, the genome-classification system 106 determines one or more of any of the sequencing metrics described above, including, but not limited to, any of one or more of the alignment metrics, depth metrics, or call-data-quality metrics described above.

As further shown in FIG. 6E, the genome-classification system 106 identifies normal genomic coordinates 646 and outlier genomic coordinates 648 based on one or more of the sequencing-metrics distribution 644. For instance, the genome-classification system 106 fits a Bayesian Gaussian mixture model to a genome-wide distribution for each of depth metrics, mapping-quality metrics, nucleobase-call-quality metrics, and/or other sequencing metrics described above across genomic coordinates. The genome-classification system 106 subsequently uses an algorithm to prune or remove components (e.g., a subset of sequencing metrics) that do not contribute or contribute little to an appropriate fit of the genome-wide distribution for each sequencing metric to the Bayesian Gaussian mixture model. Based on the fitted distribution for each sequencing metric, the genome-classification system 106 sets a p-value threshold to define or identify the normal genomic coordinates 646 that fall within the fitted distribution and the outlier genomic coordinates 648 that fall outside the fitted distribution—according to each particular sequencing metric. Accordingly, a genomic coordinate may be one of the normal genomic coordinates 646 for one sequencing metric but one of the outlier genomic coordinates 648 for another sequencing metric.

After identifying the normal genomic coordinates 646 and the outlier genomic coordinates 648, the genome-classification system 106 further identifies the genomic coordinates that exhibit normal depth metrics, mapping-quality metrics, and nucleobase-call-quality metrics as part of the high-confidence region 652. As indicated by an overlap visualization 650, the genome-classification system 106 determines the genomic coordinates that fall within a distribution (e.g., fitted distribution) for each of depth metrics, mapping-quality metrics, and nucleobase-call-quality metrics. The identified genomic coordinates form the high-confidence region 652 and comprise 89.9% of the reference genome—excluding gaps of other regions. The genomic coordinates that fall outside the distribution for any one of depth metrics, mapping-quality metrics, and nucleobase-call-quality metrics form a low-confidence region 654. As depicted in FIG. 6E, in certain embodiments, the genome-classification system 106 labels the genomic coordinates within the high-confidence region 652 with a ground-truth classification of high confidence for a somatic-nucleobase variant reflecting cancer.

As suggested above, genomic coordinates that exhibit normal depth metrics, mapping-quality metrics, and nucleobase-call-quality metrics also exhibit better accuracy or precision for determining variant-nucleobase calls. To test the reliability and further distinguish ground-truth classifications, in some embodiments, the genome-classification system 106 determines nucleobase calls for an admixture genome and compares the nucleobase calls to truthset bases unique to the genome samples forming the admixture genome from Platinum Genomes. By comparing variant calls for the admixture genome to corresponding truthset bases, the genome-classification system 106 can identify true positive variants at corresponding genomic coordinates.

Because variants in an admixture genome simulating cancer or mosaicism are so few, in some implementations, the genome-classification system 106 identifies false positive variants determined at genomic coordinates using a normal-normal subtraction method. In particular, the genome-classification system 106 determines nucleobase calls for two replicates of the same genome sample (e.g., NA12877) from the admixture—by treating one replicate as the tumor sample and another replicate as the normal sample in a tumor/normal data analysis from Illumina, Inc.—and compares the nucleobase calls from the two replicates to identify false positive variants. When executing such an analysis, for instance, the genome-classification system 106 can use the tumor/normal data analysis described by Illumina, Inc., “Evaluating Somatic Variant Calling in Tumor/Normal Studies” (2015), available at https://www.illumina.com/content/dam/illumina-marketing/documents/products/whitepapers/whitepaper_wgs_tn_somatic_variant_calling.pdf, the contents of which are hereby incorporated by reference. By measuring a density of false positive variants at genomic coordinates or genomic regions, the genome-classification system 106 can identify genomic coordinates or regions least likely to produce errors in determining nucleobase-variant calls for a given genome sample with cancer or mosaicism. In accordance with one or more embodiments, FIG. 6F illustrates a false-positive-density graph 656 depicting the density of false positives determined within the high-confidence region 652 and the low-confidence region 654 from FIG. 6E at different read depths.

In addition to determining density of false positive variants, in some embodiments, the genome-classification system 106 determines somatic-quality metrics for nucleobase calls from sample nucleic-acid sequences of an admixture genome and determines the density of false positive variants within portions of the low-confidence region 654 from FIG. 6E as partitioned by somatic-quality-metric thresholds. As explained further below, in some cases, the genome-classification system 106 uses somatic-quality-metric thresholds to distinguish different tiers of ground-truth classifications for genomic coordinates in either the low-confidence region 654 or the high-confidence region 652. In accordance with one or more embodiments, FIG. 6F further illustrates the false-positive-density graph 656 depicting the density of false positives determined within different tiers of the low-confidence region 654 from FIG. 6E at different somatic-quality-metric thresholds and at different read depths.

As shown in the false-positive-density graph 656 of FIG. 6F, the genome-classification system 106 determines a density of false positive variants per million bases (Mb) at genomic coordinates of a high-confidence region and a low-confidence region at different read depths. The genome-classification system 106 further determines the density of false positive variants in the low-confidence region according to different somatic-quality-metric thresholds—that is, somatic-quality metrics with values of 17.5, 20, and 25. For read depths of 100 at genomic coordinates, the genome-classification system 106 determines a false-positive density of just over 0.1/Mb for genomic coordinates in the high-confidence region, a false-positive density of over 1.6/Mb for genomic coordinates in the low-confidence region with a somatic-quality metric between 17.5 and 20, a false-positive density of over 0.8/Mb for genomic coordinates in the low-confidence region with a somatic-quality metric between 20 and 25, and a false-positive density of over 0.2/Mb for genomic coordinates in the low-confidence region with a somatic-quality metric over 25. For read depths of 75 at a given genomic coordinate, the genome-classification system 106 determines a false-positive density of just under 0.1/Mb for genomic coordinates in the high-confidence region, a false-positive density of over 1.1/Mb for genomic coordinates in the low-confidence region with a somatic-quality metric between 17.5 and 20, a false-positive density of over 0.7/Mb for genomic coordinates in the low-confidence region with a somatic-quality metric between 20 and 25, and a false-positive density of approximately 0.3/Mb for genomic coordinates in the low-confidence region with a somatic-quality metric over 25.

As the false-positive-density graph 656 indicates, the density of false positive variants increases as the somatic-quality metric for genomic coordinates in the low-confidence region decreases. Conversely, as the somatic-quality-metric threshold increases, the density of false positive variants decreases while the density of false negative variants increases. Because the density of false positive variants is an inverse indicator for accuracy of a somatic-variant caller, the false-positive-density graph 656 shows that the accuracy with which the genome-classification system 106 determines somatic-variant calls in terms of false positive variants increases as the somatic-quality metric for genomic coordinates in the low-confidence region decreases.

By using somatic-quality-metric thresholds, in certain implementations, the genome-classification system 106 can accordingly differentiate ground-truth classifications for genomic coordinates within a low-confidence region. For instance, in some cases, the genome-classification system 106 can label genomic coordinates from a low-confidence region with a low-confidence classification when a corresponding somatic-quality metric is below 25 and with an intermediate-confidence classification when a corresponding somatic-quality metric exceeds 25. By contrast, the genome-classification system 106 can score genomic coordinates from a low-confidence region with a lower confidence score when a corresponding somatic-quality metric is below 25 and with higher confidence score when a corresponding somatic-quality metric exceeds 25. As just set forth, a threshold of 25 for differentiating ground-truth classifications is merely an example. In additional embodiments, the genome-classification system 106 uses a different threshold or thresholds (e.g., 15, 20, 30) for somatic-quality metrics.

As further indicated by the false-positive-density graph 656 of FIG. 6F, in some embodiments, the genome-classification system 106 can use different and more stringent somatic-quality-metric thresholds for low-confidence regions to identify more reliable genomic regions among genomic regions often considered low quality by conventional systems. Conventional variant callers typically use a threshold value for somatic variant call quality. When candidate nucleobase calls that have a quality below the threshold value, conventional variant callers filter out (e.g., label as non-PASS) corresponding nucleobase calls. When threshold somatic-quality metrics increase, variant callers filter more nucleobase calls out, which results in decreasing false positive variants but increasing false negative variants. Typically, the threshold value for a somatic-quality metric used by a variant caller is chosen to achieve an optimal balance of false positive variants and false negative variants. By using the somatic-quality-metric thresholds described above to filter nucleobase calls, however, the genome-classification system 106 can significantly reduce false positive variants without excessively penalizing recall, as shown further below.

As indicated above, in certain implementations, the genome-classification system 106 determines a rate of recall for determining variant-nucleobase calls at particular genomic coordinates and generates ground-truth classifications based in part on the rate of recall. For instance, in certain cases, the genome-classification system 106 determines somatic-variant calls for an admixture of genomic samples and compares the somatic-variant calls to the truthsets (e.g., from Platinum Genomes) for the corresponding genomic samples from the admixture to determine a rate of recall. In some embodiments, the genome-classification system 106 determines a rate of recall by determining a number of correctly determined true-positive nucleobase-call variants divided by the number of all true-positive nucleobase-call variants. The genome-classification system 106 can accordingly determine and use such recall rates to identify ground-truth classifications specific to (i) somatic-nucleobase variants reflecting cancer or mosaicism or (ii) germline-nucleobase variants reflecting mosaicism.

In accordance with one or more embodiments, FIG. 6G illustrates recall graphs 658a and 658b that depict recall rates for the genome-classification system 106 determining somatic-nucleobase variants that reflect cancer at genomic coordinates within different genomic regions and at different variant allele frequencies. In particular, the recall graphs 658a and 658b show recall rates at 100 read depth and 75 read depth, respectively, for genomic coordinates within a high-confidence region and within a low-confidence region partitioned according to somatic-quality-metric thresholds of 17.5, 20, and 25—across different variant allele frequencies.

As indicated by the recall graphs 658a and 658b respectively for read depths of 100 and 75 at a given genomic coordinate, the genome-classification system 106 determines a rate of recall for determining somatic variants reflecting cancer at various genomic coordinates and across various variant allele frequencies. As shown in both the recall graphs 658a and 658b, genomic coordinates within the high-confidence region exhibit a higher rate of recall across variant allele frequencies than any of the partitioned low-confidence regions. Because nucleobase variants with variant allele frequencies of 0.05 to 0.2 are present in relatively fewer reads at a given genomic coordinate, a sequencing system lacks sufficient reads (even at read depths of 100 and 75 for a genomic coordinate) to determine the corresponding nucleobase-variant calls in the high-confidence region at the nearly 1.0 rate of recall exhibited at higher variant allele frequencies.

As further shown in both the recall graphs 658a and 658b, genomic coordinates in each of the low-confidence region with a somatic-quality-metric of 25, the low-confidence region with a somatic-quality-metric threshold of 20, and the low-confidence region with a somatic-quality-metric threshold of 17.5 exhibit increasingly better rates of recall across variant allele frequencies. In other words, as somatic-quality-metric thresholds for filtering increase for genomic coordinates, the rate of recall for determining somatic variants reflecting cancer decreases for genomic coordinates. Note that this relationship between somatic-quality-metric thresholds and the rate of recall is not representative of somatic-quality metric increases. As somatic-quality metrics increase, the rate of recall for determining somatic variants should likewise increases, and somatic variant calls are less prone to both false negative variants and false positive variants.

By using both somatic-quality-metric thresholds and recall rates, in certain implementations, the genome-classification system 106 can accordingly differentiate ground-truth classifications for genomic coordinates within a low-confidence region. For instance, in some cases, the genome-classification system 106 labels genomic coordinates from a low-confidence region with a low-confidence classification when a corresponding somatic-quality metric is below 25 (or some other somatic-quality-metric threshold). Conversely, the genome-classification system 106 labels genomic coordinates from a low-confidence region with an intermediate-confidence classification when a corresponding somatic-quality metric exceeds 25 (or some other somatic-quality-metric threshold). By contrast, the genome-classification system 106 can score genomic coordinates from a low-confidence region with a lower (or higher) confidence score when a corresponding somatic-quality metric is above or below 25.

By contrast, in some embodiments, the genome-classification system 106 can differentiate ground-truth classifications for genomic coordinates in a low-confidence region based on the F-scores of genomic coordinates with different somatic-quality-metric thresholds. For example, the genome-classification system 106 can determine F-scores for determining variant-nucleobase calls at genomic coordinates in the low-confidence region based on both a rate of recall and a rate of precision. In some embodiments, the genome-classification system 106 determines a rate of precision by determining a number of correctly determined true-positive nucleobase-call variants divided by the number of all determined nucleobase-call variants. In some cases, the genome-classification system 106 determines an F₁ score by determining a harmonic mean of the rate of precision and the rate of recall. Accordingly, the genome-classification system 106 can label genomic coordinates in the low-confidence region—that have different somatic-quality-metric thresholds—with different ground-truth classifications depending on the corresponding F-scores of the genomic coordinates with different somatic-quality-metric thresholds.

As further indicated above, in certain implementations, the genome-classification system 106 determines one or both of a rate of precision and a rate of recall for determining variant-nucleobase calls at particular genomic coordinates and generates ground-truth classifications based on one or both of the rate of precision and the rate of recall. For instance, in certain cases, the genome-classification system 106 determines somatic-variant calls for an admixture of genomic samples (e.g., by using a tumor/normal DRAGEN Somatic Pipeline when determining somatic-variant calls simulating cancer or using a tumor-only analysis in DRAGEN Somatic Pipeline when determining somatic-variant calls simulating mosaicism). The genome-classification system 106 subsequently compares the somatic-variant calls to the truthsets (e.g., from Platinum Genomes) for the corresponding genomic samples from the admixture to determine rates of precision and recall. The genome-classification system 106 can accordingly determine and use such precision or recall rates to identify ground-truth classifications specific to (i) somatic-nucleobase variants reflecting cancer or mosaicism or (ii) germline-nucleobase variants reflecting mosaicism.

In accordance with one or more embodiments, FIG. 6H illustrates precision graphs 660a and 660b that depict the precision with which the genome-classification system 106 determines variant-nucleobase calls reflecting mosaicism at genomic coordinates within different genomic regions and at different variant allele frequencies. FIG. 6H further illustrates recall graphs 662a and 662b that depict recall rates for the genome-classification system 106 determining nucleobase variants reflecting mosaicism at genomic coordinates within different genomic regions and at different variant allele frequencies.

As indicated by the precision graphs 660a and 660b respectively for read depths of 100 and 75 at a given genomic coordinate, the genome-classification system 106 determines a rate of precision for determining nucleobase variants reflecting mosaicism at various genomic coordinates and across various variant allele frequencies. As shown in both the precision graphs 660a and 660b, genomic coordinates within the high-confidence region generally exhibit a higher rate of precision across variant allele frequencies than genomic coordinates within the low-confidence region. Starting at a variant allele frequency of 0.15 in both the precision graphs 660a and 660b, genomic coordinates within the low-confidence region exhibit nearly the same rate of precision of nearly 1.000 as genomic coordinates within the high-confidence region.

As indicated by the recall graphs 662a and 662b respectively for read depths of 100 and 75 at a given genomic coordinate, the genome-classification system 106 determines a rate of recall for determining nucleobase variants reflecting mosaicism at various genomic coordinates and across various variant allele frequencies. As shown in both the recall graphs 662a and 662b, genomic coordinates within the high-confidence region consistently exhibit a higher rate of recall across variant allele frequencies than genomic coordinates within the low-confidence region.

As suggested above, nucleobase variants with variant allele frequencies of 0.05 to 0.15 are present in relatively fewer nucleotide reads at a given genomic coordinate. Accordingly, a sequencing system lacks sufficient reads (even at read depths of 100 and 75 for a genomic coordinate) to determine the corresponding nucleobase-variant calls with the nearly 1.0 rate of precision or the nearly 1.0 rate of recall exhibited at higher variant allele frequencies.

In addition to determining rates of precision and recall, in certain implementations, the genome-classification system 106 further determines F-scores for determining variant-nucleobase calls at genomic coordinates based on the rates of precision and recall. As indicated above, in some cases, the genome-classification system 106 determines an F₁ score by determining a harmonic mean of the rate of precision and the rate of recall. Accordingly, the genome-classification system 106 can label genomic coordinates or genomic regions, such as the high-confidence region and the low-confidence region, with different ground-truth classifications according to relative F₁ scores.

Based on one or both of recall rates and precision rates, in certain implementations, the genome-classification system 106 differentiates ground-truth classifications for genomic coordinates within the high-confidence region and the low-confidence region. For instance, in some cases, the genome-classification system 106 labels genomic coordinates in the high-confidence region with high-confidence classifications in part because genomic coordinates in the high-confidence region exhibit better recall rates and precision rates. By contrast, in some cases, the genome-classification system 106 labels genomic coordinates in the low-confidence region with low-confidence classifications (or intermediate-confidence classifications) because the low-confidence region exhibits lower recall rates and precision rates.

Regardless of how the genome-classification system 106 determines or labels such ground-truth classifications, in certain cases, the genome-classification system 106 trains the genome-location-classification model 608 to determine, for somatic-nucleobase variants reflecting cancer or somatic mosaicism or for germline-nucleobase variants reflecting germline mosaicism, variant confidence classifications for genomic coordinates based on such determined ground-truth classifications as depicted in FIG. 6A. Accordingly, the genome-classification system 106 can likewise utilize a trained version of the genome-location-classification model 608 to determine variant confidence classifications that are both for a set of genomic coordinates and specific to somatic-nucleobase variants reflecting cancer or somatic mosaicism or for germline-nucleobase variants reflecting germline mosaicism, as depicted in FIG. 6B. Consequently, the genome-classification system 106 can also identify and display variant confidence classifications from the trained version of the genome-location-classification model 608 corresponding to genomic coordinates of variant calls somatic-nucleobase variants reflecting cancer or somatic mosaicism or for germline-nucleobase variants reflecting germline mosaicism, as depicted in FIG. 6C.

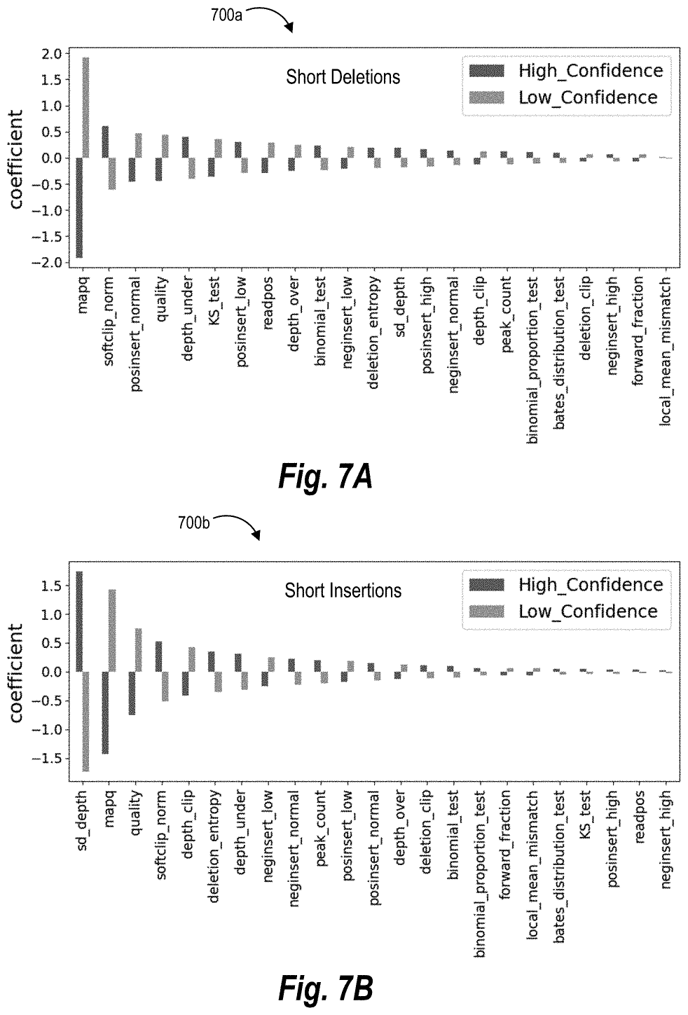

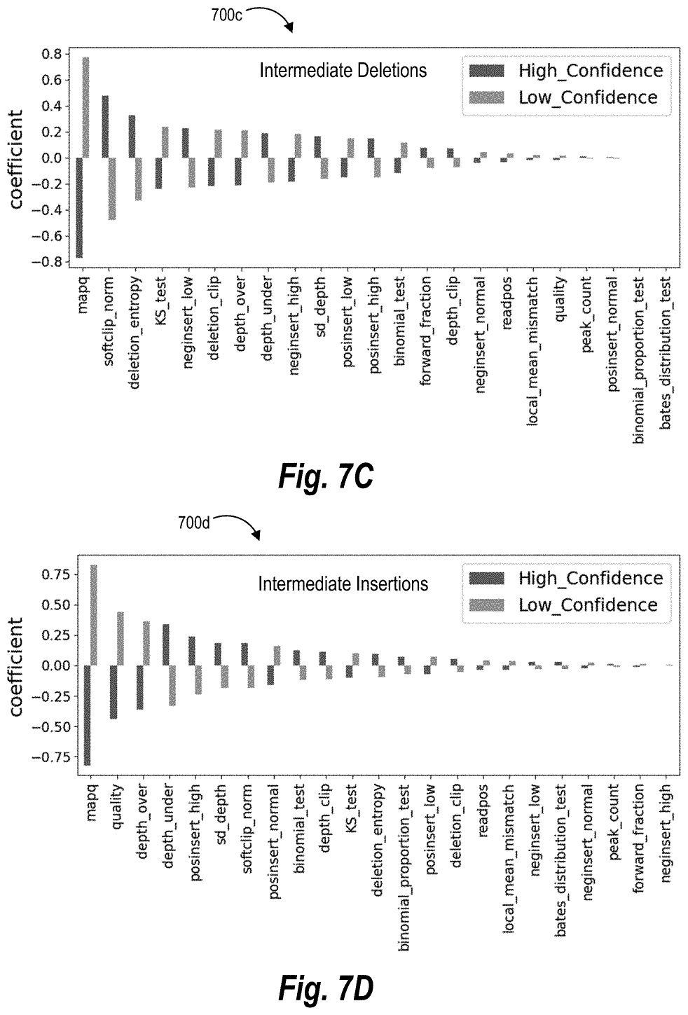

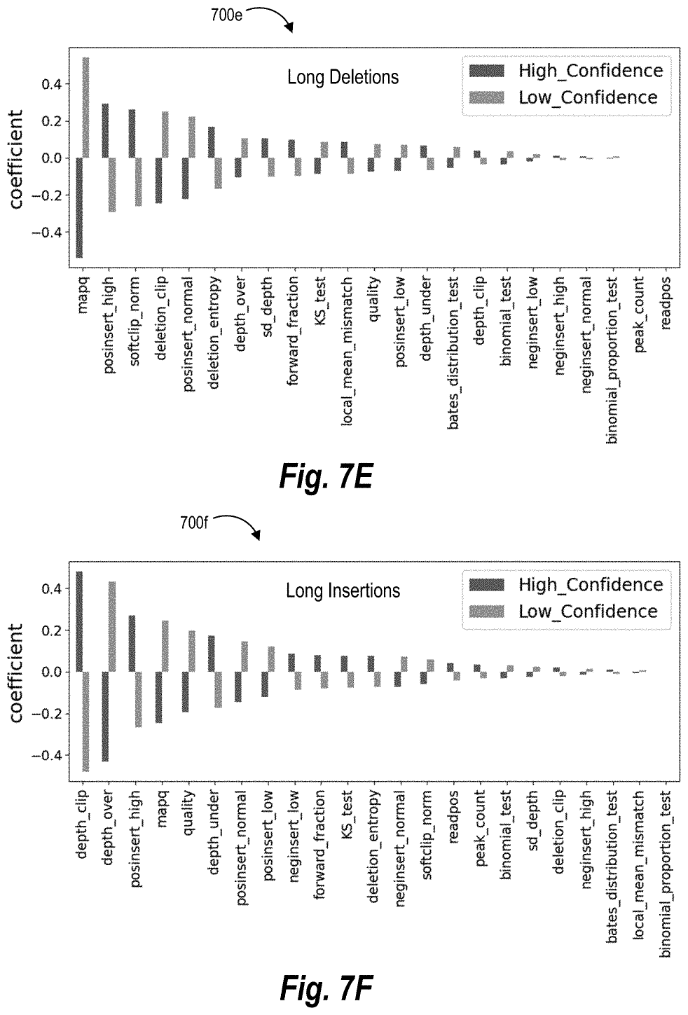

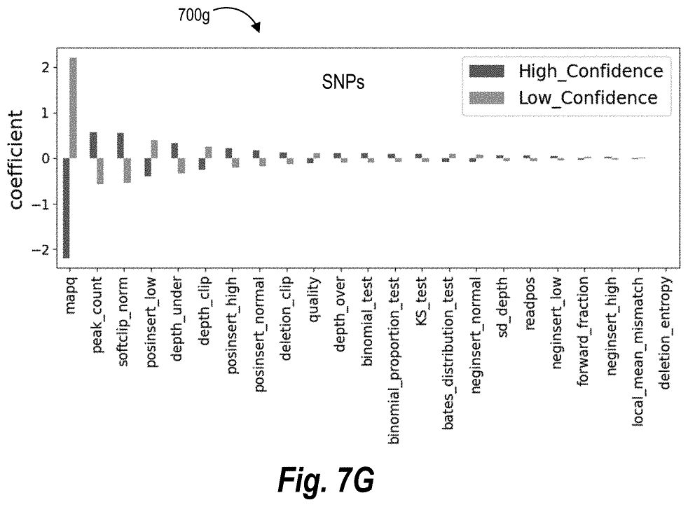

As indicated above, to assess the performance of different embodiments of a genome-location-classification model, researchers measured variables and various accuracy metrics demonstrated by confidence classifications of the genome-classification system 106. The following paragraphs describe some of those measurements as depicted in FIGS. 7-10B. In accordance with one or more embodiments, for instance, FIGS. 7A-7G depict graphs 700a-700g indicating sequencing metrics and sequencing-metric-derived-input data that inform a genome-location-classification model for specific variant types when trained from a logistic regression model. In particular, the graphs 700a-700g show the logistic regression coefficients used by a genome-location-classification model for the top twenty three sequencing metrics and sequencing-metric-derived-input data to determine high-confidence classifications or low-confidence classifications for genomic coordinates based on different nucleobase-call-variant types.

As shown in FIGS. 7A and 7B, for example, the graphs 700a and 700b show logistic regression coefficients for genome-location-classification models respectively trained using ground-truth classifications corresponding to either short deletions of 1-5 nucleobases in length (for the graph 700a) or short insertions of 1-5 nucleobases in length (for the graph 700b). FIGS. 7A and 7B show that show that the logistic regression models trained using short deletions or short insertions weight mapping-quality metrics (MAPA) or standardized depth with a coefficient of highest magnitude in comparison to other data inputs to determine high-confidence classifications or low-confidence classifications for genomic coordinates or genomic regions.

In particular, the graph 700a in FIG. 7A shows that the logistic regression model trained for short deletions uses a coefficient over −1.5 and a coefficient over 1.5 for mapping-quality metrics to determine high-confidence classifications and low-confidence classifications, respectively, for genomic coordinates or genomic regions. The graph 700b in FIG. 7B shows that the logistic regression model trained for short insertions uses a coefficient over −1.5 and a coefficient over 1.5 for standardized depth metrics to determine high-confidence classifications and low-confidence classifications, respectively, for genomic coordinates or genomic regions. Such standardized depth metrics are subject to a standard deviation and could include forward-reverse-depth metrics or normalized-depth metrics.

By contrast, the graph 700a in FIG. 7A shows that the logistic regression model trained for short deletions uses coefficients of 0.0 and coefficients of nearly 0.0—which are lower in magnitude than other data inputs for short deletions—for forward-fraction metrics and local mean of read-reference-mismatch metrics (local_mean_mismatch) to determine high-confidence classifications and low-confidence classifications for genomic coordinates. The graph 700b in FIG. 7B shows that the logistic regression model trained for short insertions uses coefficients of nearly 0.0—which are lower in magnitude than other data inputs for short insertions—for higher negative-insert-size metrics to determine high-confidence classifications and low-confidence classifications for genomic coordinates.

As shown in FIGS. 7C and 7D, the graphs 700c and 700d show logistic regression coefficients for genome-location-classification models respectively trained using ground-truth classifications corresponding to either intermediate deletions of 5-15 nucleobases in length (for the graph 700c) or intermediate insertions of 5-15 nucleobases in length (for the graph 700d). Both the graphs 700c and 700d show that the logistic regression models weight mapping-quality metrics (MAPQ) with a coefficient of highest magnitude in comparison to other data inputs to determine high-confidence classifications or low-confidence classifications for genomic coordinates or genomic regions.

In particular, the graph 700c in FIG. 7C shows that the logistic regression model trained for intermediate deletions uses a coefficient of nearly −0.8 in magnitude and nearly 0.8 in magnitude for mapping-quality metrics to determine high-confidence classifications and low-confidence classifications, respectively, for genomic coordinates. Similarly, the graph 700d in FIG. 7D shows that the logistic regression model trained for intermediate insertions uses a coefficient of over −0.75 in magnitude and over 0.75 in magnitude for mapping-quality metrics to determine high-confidence classifications and low-confidence classifications, respectively, for genomic coordinates.

By contrast, the graph 700c in FIG. 7C shows that the logistic regression model trained for intermediate deletions uses coefficients of 0.0—which are lower in magnitude than the other data inputs for intermediate deletions—for both a binomial proportion test and a Bates distribution test to determine high-confidence classifications and low-confidence classifications, respectively, for genomic coordinates. The graph 700d in FIG. 7D shows that the logistic regression model trained for intermediate insertions uses coefficients of 0.0 and nearly 0.0—which are lower in magnitude than the other data inputs for intermediate insertions—for forward-fraction metrics and higher negative-insert-size metrics to determine high-confidence classifications and low-confidence classifications, respectively, for genomic coordinates.

As shown in FIGS. 7E and 7F, the graphs 700e and 700f show logistic regression coefficients for genome-location-classification models respectively trained using ground-truth classifications corresponding to either long deletions of more than 15 nucleobases in length (for the graph 700e) or long insertions of more than 15 nucleobases in length (for the graph 700f). FIGS. 7E and 7F show that show that the logistic regression models trained using long deletions or long insertions weight mapping-quality metrics (MAPQ) or depth-clip metrics with coefficients of highest magnitude in comparison to other data inputs to determine high-confidence classifications or low-confidence classifications for genomic coordinates or genomic regions.

In particular, the graph 700e in FIG. 7E shows that the logistic regression model trained for long deletions uses coefficients over −0.4 and over 0.4 for mapping-quality metrics (MAPQ) to determine high-confidence classifications and low-confidence classifications, respectively, for genomic coordinates or genomic regions. The graph 700f in FIG. 7F shows that the logistic regression model trained for long insertions uses a coefficient of over −0.4 in magnitude and over 0.4 in magnitude for depth-clip metrics to determine high-confidence classifications and low-confidence classifications, respectively, for genomic coordinates or genomic regions.

By contrast, the graph 700e in FIG. 7E shows that the logistic regression model trained for long deletions uses coefficients of 0.0—which are lower than other data inputs for long deletions—for both peak-count metrics and read-position metrics to determine high-confidence classifications and low-confidence classifications for genomic coordinates. The graph 700f in FIG. 7F shows that the logistic regression model trained for long insertions uses coefficients of nearly 0.0 and coefficients of 0.0—which are lower than other data inputs for long insertions—for local mean of read-reference-mismatch metrics (local_mean_mismatch) and binomial proportion tests to determine high-confidence classifications and low-confidence classifications for genomic coordinates.

As shown in FIG. 7G, the graph 700g shows logistic regression coefficients for a genome-location-classification model trained using ground-truth classifications corresponding to SNPs. As shown in FIG. 7G, the graph 700g shows that the logistic regression model trained for SNPs uses a coefficient over −2.0 and a coefficient over 2.0—which are higher than the other data inputs for SNPs—for mapping-quality metrics (MAPA) to determine high-confidence classifications and low-confidence classifications, respectively, for genomic coordinates or genomic regions. By contrast, the graph 700g shows that the logistic regression model trained for SNPs uses coefficients—which are lower than the other data inputs for SNPs—for deletion-entropy metrics to determine high-confidence classifications and low-confidence classifications for genomic coordinates or genomic regions.

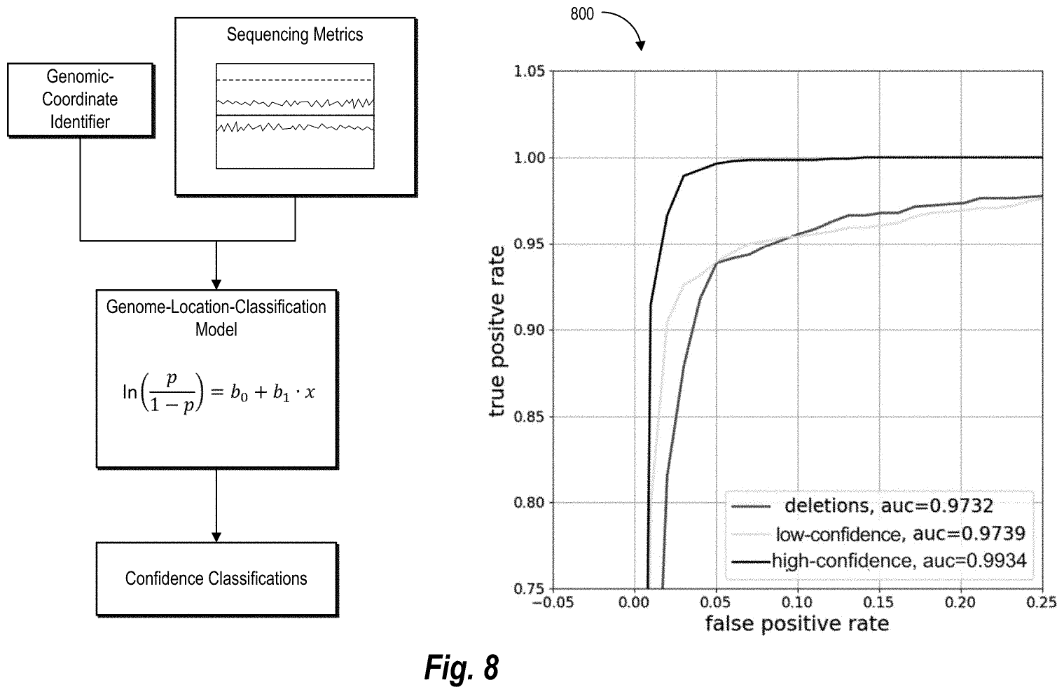

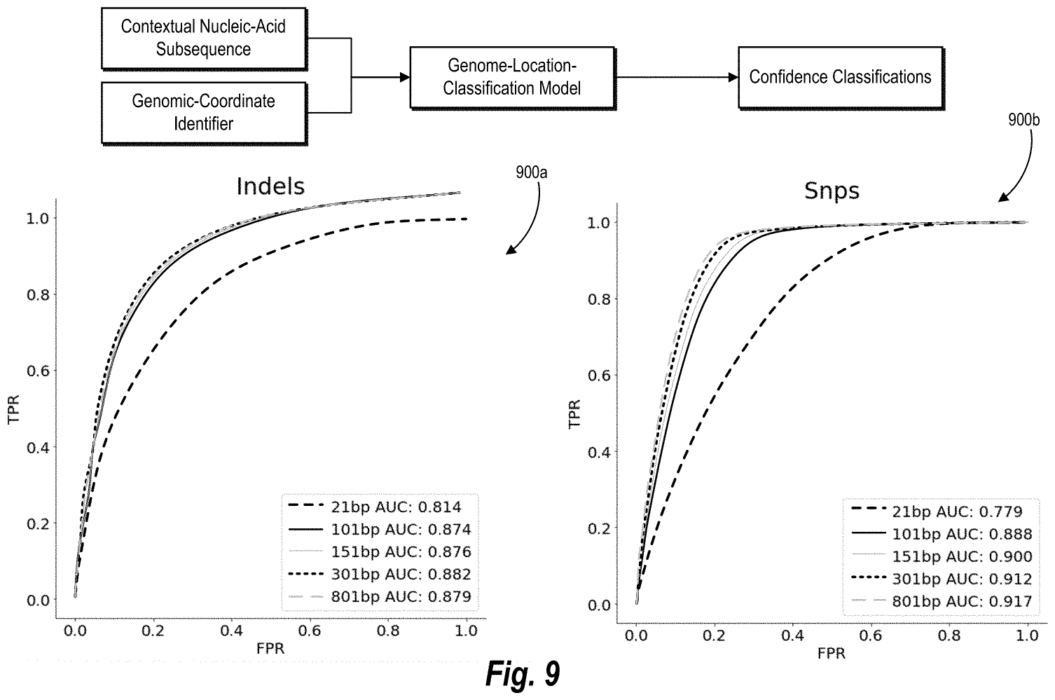

To further assess the performance of a logistic regression model trained as a genome-location-classification model based on sequencing metrics, researchers determined the rate at which such a genome-location-classification model correctly determines confidence classifications. In accordance with one or more embodiments, FIG. 8 illustrates a graph 800 with receiver operating characteristics (ROC) curves defining an area under curve (AUC) for the rate at which a logistic regression model trained as a genome-location-classification model correctly (i) determines high-confidence classifications or low-confidence classifications at genomic coordinates as true positives or false positives and (ii) determines confidence classifications as true positives and false positives for genomic coordinates with common deletions. As shown in FIG. 8, the genome-classification system 106 inputs data derived or prepared from sequencing metrics into the genome-location-classification model to determine confidence classifications for genomic coordinates.

As indicated by the graph 800, a logistic regression model trained as a genome-location-classification model correctly determines high-confidence classifications as true positives or false positives for genomic coordinates with an AUC of 99.34% based on comparisons with ground-truth classifications. As further indicated by the graph 800, such a genome-location-classification model correctly determines low-confidence classifications as true positives or false positives for genomic coordinates with an AUC of 97.39% based on comparisons with ground-truth classifications. Finally, such a genome-location-classification model correctly determines confidence classifications as true positives or false positives for genomic coordinates at which common deletions occur with an AUC of 97.32% based on comparisons with a reference genome.

In addition to determining the ROC curves for the graph 800 depicted in FIG. 8, researchers also assessed the precision, recall, and concordance (or reproducibility) with which a variant caller can identify SNVs and indels at genomic coordinates classified by a logistic regression model trained as a genome-location-classification model. Various tests demonstrate that a logistic regression model trained as a genome-location-classification model correctly classifies a larger portion of the human genome with high-confidence coordinates (or regions) at which SNVs and indels can be correctly identified than those identified by GIAB. Indeed, such a genome-location-classification model can identify certain genomic coordinates (or regions) with a high-confidence classification that GIAB identifies as within a difficult region. Table 2 below, for instance, demonstrates that the genome-classification system 106 improves the accuracy with which existing sequencing systems identify a degree of confidence at which nucleobases can be determined at specific genomic coordinates.

TABLE 2

| | % Non-N | | | | |
|---|---|---|---|---|---|
| | austosomal | Variant | Precision | Recall | Concordance |
| GIAB | | | | | |
| Not Difficult | 79.0% | SNVs | >99.9% (99.9%- | >99.9% (>99.9%- | 99.8% (99.5%- |
| | | | >99.9%) | >99.9%) | 99.8%) |
| | | Indels | 99.7% (99.7%- | 99.9% (99.9%- | 98.9% (98.5%- |
| | | | 99.8%) | 99.9%) | 99.0%) |
| Difficult | 21.0% | SVs | 99.1% (99.0%- | 96.8% (96.5%- | 82.9% (82.3%- |
| | | | 99.2%) | 97.1%) | 83.3%) |
| | | Indels | 97.2% (97.0%- | 98.2% (98.1%- | 87.6% (85.9%- |
| | | | 97.3%) | 98.3%) | 88.3%) |
| Genome- | | | | | |
| Classification | | | | | |
| System | | | | | |
| High | 90.3% | SNVs | >99.9% (99.9%- | 99.9% (99.9%- | 99.9% (99.8%- |
| | | | >99.9%) | >99.9%) | 99.9%) |
| Confidence | | Indels | 99.0% (98.7%- | 99.5% (99.4%- | 98.5% (98.2%- |
| | | | 99.1%) | 99.5%) | 98.7%) |
| Intermediate | 2.9% | SNVs | 99.3% (99.2%- | 98.4% (98.2%- | 97.8% (97.5%- |
| | | | 99.5%) | 98.6%) | 98.0%) |
| Confidence | | Indels | 90.3% (89.9%- | 96.8% (96.4%- | 87.7% (87.1%- |
| | | | 90.7%) | 97.0%) | 88.1%) |
| Low | 4.9% | SNVs | 95.2% (94.7%- | 82.3% (80.3%- | 79.0% (77.1%- |
| | | | 95.6%) | 83.8%) | 80.7%) |
| Confidence | | Indels | 74.4% (72.7%- | 74.5% (71.3%- | 59.3% (56.2%- |
| | | | 75.5%) | 77.2%) | 61.2%) |
| Common | 1.9% | SNVs | 97.1% (96.9%- | 90.9% (90.4%- | 88.5% (87.9%- |
| | | | 97.4%) | 91.3%) | 89.1%) |
| Deletions | | Indels | 96.7% (96.5%- | 98.3% (98.2%- | 95.1% (94.9%- |
| | | | 96.8%) | 98.4%) | 95.2%) |

As shown in Table 2, a logistic regression model trained as a genome-location-classification model correctly classifies genomic coordinates at 90.3% of the non-N autosomal human genome. By contrast, GIAB has identified genomic regions at which variants can be accurately determined without difficulty in only 79-84% of the non-N autosomal human genome. As further indicated by Table 2, such a logistic regression model accurately classifies genomic coordinates with approximately 99.9% precision, 99.9% recall, and 99.9% concordance based on ground-truth classifications determined using SNV data. Similarly, such a logistic regression model accurately classifies genomic coordinates with approximately 99.0% precision, 99.5% recall, and 98.5% concordance based on ground-truth classifications determined using indel data. At genomic coordinates labeled with an intermediate-confidence classification or a low-confidence classification by such a logistic regression model—or genomic regions comprising common deletions—such a logistic regression model classifies genomic coordinates based on ground-truth data derived from SNVs or indels with lower precision, recall, and concordance rates further reported in Table 2.

To assess the performance of a CNN trained as a genome-location-classification model based on contextual nucleic-acid subsequences, researchers determined the rate at which a such a genome-location-classification model correctly determines confidence classifications. In accordance with one or more embodiments, FIG. 9 illustrates a graph 900a with ROC curves defining an AUC for a CNN trained as a genome-location-classification model determining confidence classifications for genomic coordinates based on ground-truth classifications derived from indel data. FIG. 9 further illustrates a graph 900b with ROC curves defining an AUC for a CNN trained as a genome-location-classification model determining confidence classifications for genomic coordinates based on ground-truth classifications derived from data for single nucleotide polymorphisms (SNPs). As shown in FIG. 9, to determine confidence classifications for genomic coordinates, the genome-classification system 106 inputs data derived or prepared from contextual nucleic-acid subsequences into the CNN trained as a genome-location-classification model.

As an overview, the graphs 900a and 900b demonstrate that a CNN trained as a genome-location-classification model correctly determines confidence classifications for genomic coordinates as true positives or false positives based on ground-truth data derived from indels or SNPs with an AUC between 77.9% and 91.7%—depending on the length of the contextual nucleic-acid subsequences input into the genome-location-classification model. In particular, as indicated by the graph 900a, the genome-location-classification model trained for indels correctly determines confidence classifications for genomic coordinates as true positives or false positives with an AUC 81.4%, 87.4%, 87.6%, 88.2%, and 87.9% based on contextual nucleic-acid subsequences of 21 base pairs, 101 base pairs, 151 base pairs, 301 base pairs, and 801 base pairs, respectively. As indicated by the graph 900b, the genome-location-classification model trained for SNPs correctly determines confidence classifications for genomic coordinates as true positives or false positives with an AUC of 77.9%, 88.8%, 90.0%, 91.2%, and 91.7% based on contextual nucleic-acid subsequences of 21 base pairs, 101 base pairs, 151 base pairs, 301 base pairs, and 801 base pairs, respectively. For both indels and SNPs, therefore, a CNN trained as the genome-location-classification model more accurately determines confidence classifications for genomic coordinates as the length of the contextual nucleic-acid subsequence increases for the confidence classifications.

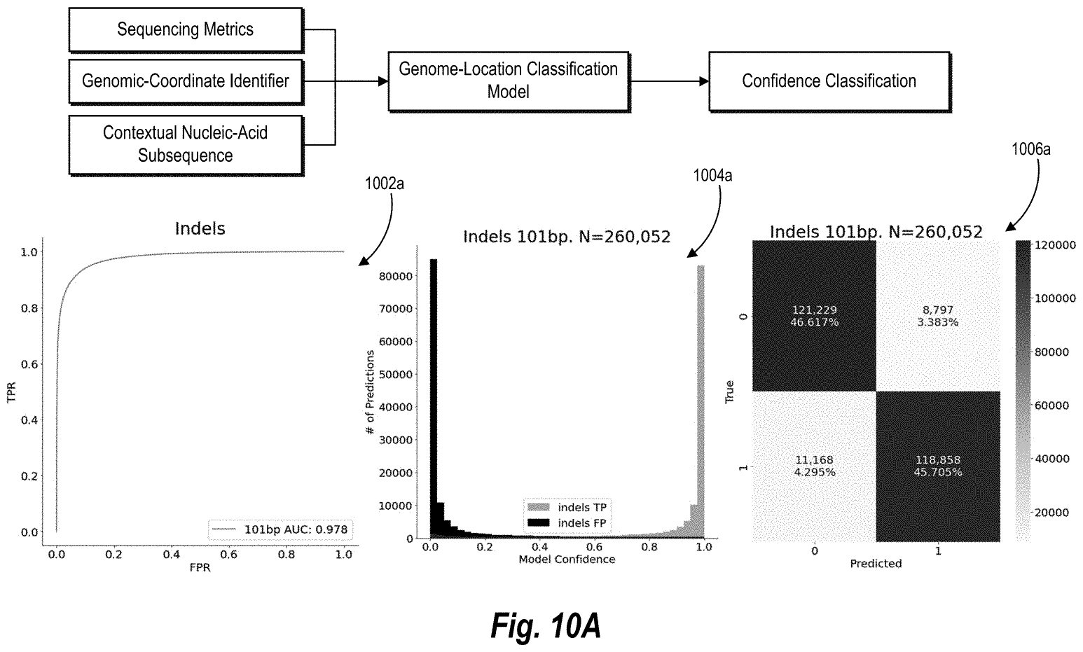

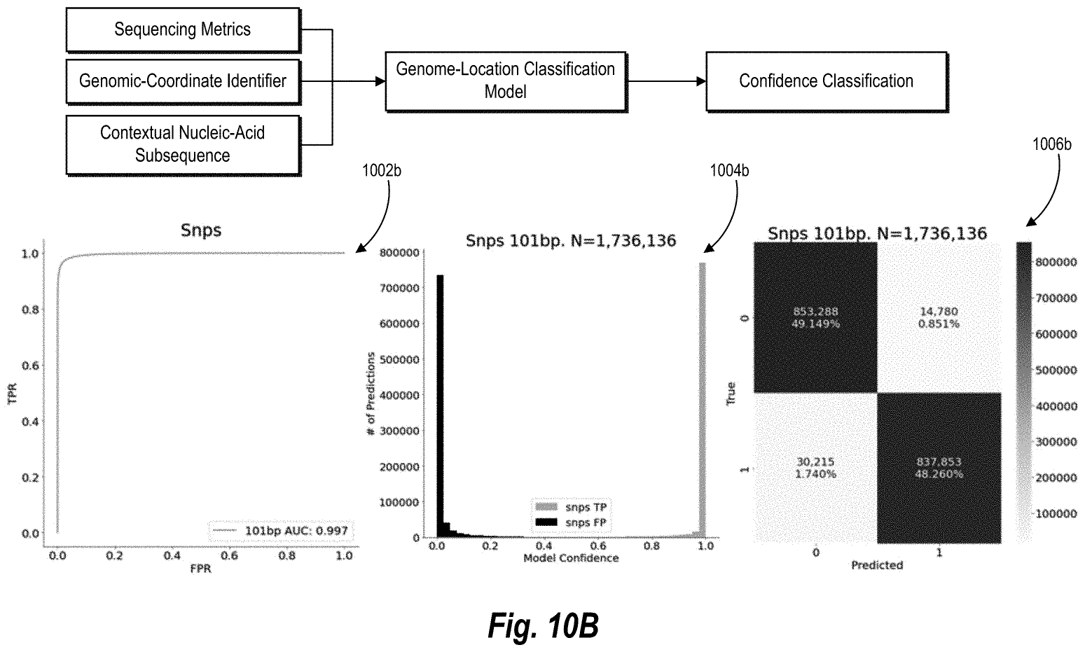

To test the performance of a CNN trained as a genome-location-classification model based on both sequencing metrics and contextual nucleic-acid subsequences, researchers also determined the rate at which such a genome-location-classification model correctly determines confidence classifications using a testing or hold-out dataset. In accordance with one or more embodiments, FIGS. 10A and 10B illustrate graphs 1002a-1002b, histograms 1004a-1004b, and confusion matrices 1006a-1006b depicting rates and confidences at which such a genome-location-classification model correctly determines confidence classifications for particular genomic coordinates based on ground-truth classifications derived from indels and SNP data. As shown in FIGS. 10A and 10B, to determine confidence classifications for genomic coordinates, the genome-classification system 106 inputs data derived (or prepared) from both sequencing metrics and contextual nucleic-acid subsequences into the CNN trained as the genome-location-classification model.

As indicated by the graph 1002a in FIG. 10A, a CNN trained for indels as a genome-location-classification model correctly determines confidence classifications as true positives or false positives for genomic coordinates with an AUC of 97.8% based on contextual nucleic-acid subsequences of 101 base pairs. As indicated by the graph 1002b in FIG. 10B, a CNN trained for SNPs as a genome-location-classification model correctly determines confidence classifications as true positives or false positives for genomic coordinates with an AUC of 99.7% based on contextual nucleic-acid subsequences of 101 base pairs. Accordingly, the graphs 1002a and 1002b demonstrate that a CNN trained as a genome-location-classification model as shown in FIGS. 10A and 10B can correctly determine confidence classifications for specific genomic coordinates at extraordinarily high rates when using both sequencing metrics and contextual nucleic-acid subsequences as inputs.

Turning back now to the histogram 1004a in FIG. 10A for indels. As indicated by the histogram 1004a, a CNN trained for indels as a genome-location-classification model correctly determines confidence classifications as true positives in over 80,000 predictions with a confidence of approximately 1.0 at genomic coordinates. In other words, based on contextual nucleic-acid subsequences of 101 base pairs, such a genome-location-classification model determines classifications with high confidence at genomic coordinates at which a true-positive indel is detected. As further indicated by the histogram 1004a, a CNN trained for indels as a genome-location-classification model correctly determines confidence classifications as false positives with a confidence of approximately 0.0 in over 80,000 predictions at genomic coordinates. In other words, based on contextual nucleic-acid subsequences of 101 base pairs, such a genome-location-classification model determines classifications with low confidence at genomic coordinates at which a false-positive indel is detected.

Turning back now to the histogram 1004b in FIG. 10B for SNPs. As indicated by the histogram 1004b, a CNN trained for SNPs as a genome-location-classification model correctly determines confidence classifications as true positives in nearly 800,000 predictions with a confidence of approximately 1.0 at genomic coordinates. In other words, based on contextual nucleic-acid subsequences of 101 base pairs, the genome-location-classification model determines classifications with high confidence at genomic coordinates at which a true-positive SNP is detected. As further indicated by the histogram 1004b, a CNN trained for SNPs as a genome-location-classification model correctly determines confidence classifications as false positives in over 700,000 predictions with a confidence of approximately 0.0 at genomic coordinates. In other words, based on contextual nucleic-acid subsequences of 101 base pairs, the genome-location-classification model determines classifications with low confidence at genomic coordinates at which a false-positive SNP is detected.

Turning back now to the confusion matrices 1006a and 1006b in FIGS. 10A and 10B. As depicted by the confusion matrix 1006a in FIG. 10A, a CNN trained for indels as a genome-location-classification model correctly determines confidence classifications as true positives (e.g., high-confidence classification) or true negatives (e.g., low-confidence classification) at a rate of 92.322% from total predictions at genomic coordinates. By contrast, such a CNN a sequencing system incorrectly determines confidence classifications as true positives or true negatives only at a rate of 7.678% from total predictions at genomic coordinates. As depicted by the confusion matrix 1006b in FIG. 10B, a CNN trained for SNPs as a genome-location-classification model correctly determines confidence classifications as true positives or true negatives at a rate of 97.409% from total predictions at genomic coordinates. By contrast, such a CNN incorrectly determines confidence classifications as true positives or true negatives only at a rate of 2.591% from total predictions at genomic coordinates.

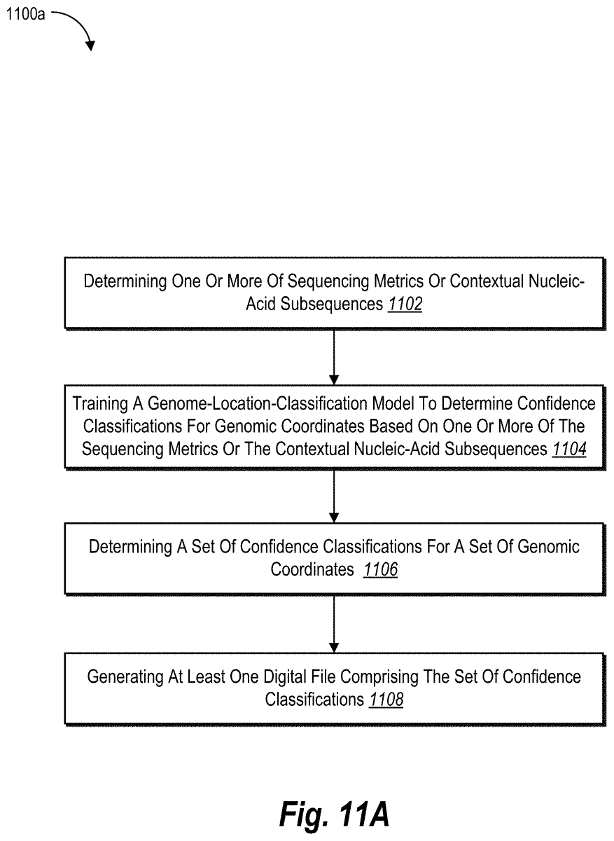

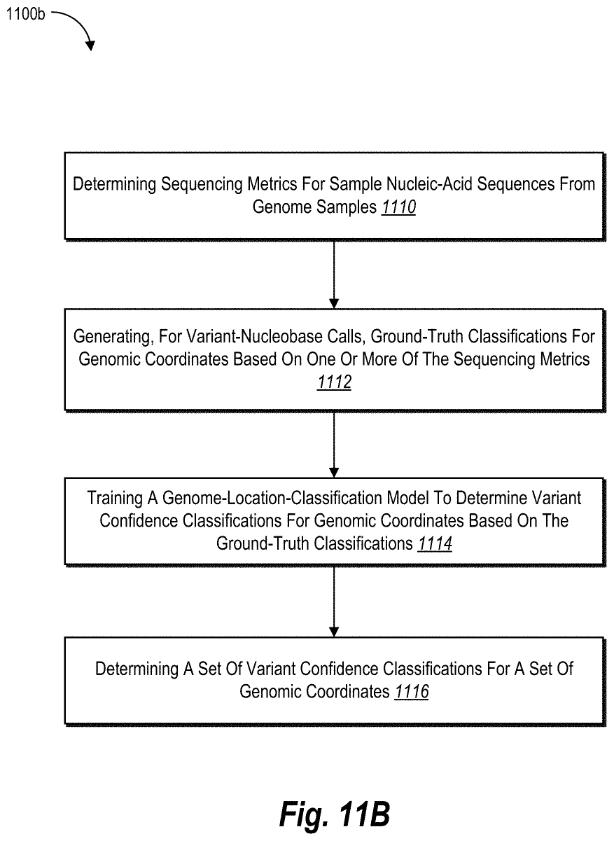

Turning now to FIG. 11A, this figure illustrates a flowchart of a series of acts 1100a of training a machine-learning model to determine confidence classifications for genomic coordinates in accordance with one or more embodiments. While FIG. 11A illustrates acts according to one embodiment, alternative embodiments may omit, add to, reorder, and/or modify any of the acts shown in FIG. 11A. The acts of FIG. 11A can be performed as part of a method. Alternatively, a non-transitory computer readable storage medium can comprise instructions that, when executed by one or more processors, cause a computing device to perform the acts depicted in FIG. 11A. In still further embodiments, a system comprising at least one processor and a non-transitory computer readable medium comprising instructions that, when executed by one or more processors, cause the system to perform the acts of FIG. 11A.

As shown in FIG. 11A, the acts 1100a include an act 1102 of determining one or more of sequencing metrics or contextual nucleic-acid subsequences. In particular, in some embodiments, the act 1102 includes determining sequencing metrics for comparing sample nucleic-acid sequences with genomic coordinates of an example nucleic-acid sequence. In some cases, the act 1102 comprises determining, from an example nucleic-acid sequence, a contextual nucleic-acid subsequence surrounding a variant-nucleobase call in a sample nucleic-acid sequence at a genomic coordinate from genomic coordinates of a reference genome. In one or more embodiments, the sample nucleic-acid sequences are determined using a single sequencing pipeline comprising a nucleic-acid-sequence-extraction method, a sequencing device, and a sequence-analysis software. Relatedly, in certain embodiments, the example nucleic-acid sequence comprises a reference genome or a nucleic-acid sequence of an ancestral haplotype.

As indicated above, in some cases, determining the sequencing metrics comprises determining one or more of: alignment metrics for quantifying alignment of the sample nucleic-acid sequences with the genomic coordinates of the example nucleic-acid sequence; depth metrics for quantifying depth of nucleobase calls for the sample nucleic-acid sequences at the genomic coordinates of the example nucleic-acid sequence; or call-data-quality metrics for quantifying quality of the nucleobase calls for the sample nucleic-acid sequences at the genomic coordinates of the example nucleic-acid sequence.

Relatedly, in certain implementations, determining the alignment metrics comprises determining one or more of deletion-size metrics, mapping-quality metrics, positive-insert-size metrics, negative-insert-size metrics, soft-clipping metrics, read-position metrics, or read-reference-mismatch metrics for the sample nucleic-acid sequences; determining the depth metrics comprises determining one or more of forward-reverse-depth metrics or normalized-depth metrics; or determining the call-data-quality metrics comprises determining one or more of nucleobase-call-quality metrics or callability metrics for the sample nucleic-acid sequences.

As further shown in FIG. 11A, the acts 1100a include an act 1104 of training a genome-location-classification model to determine confidence classification for genomic coordinates based on one or more of the sequencing metrics or the contextual nucleic-acid subsequences. In particular, in some embodiments, the act 1104 includes training a genome-location-classification model to determine confidence classifications for the genomic coordinates based on the sequencing metrics and ground-truth classifications for particular genomic coordinates. Further, in some cases, the act 1104 includes training a genome-location-classification model to determine confidence classifications for the genomic coordinate based on the contextual nucleic-acid subsequence and a ground-truth classification for the genomic coordinate.

As suggested above, in certain embodiments, training the genome-location-classification model to determine the confidence classifications comprises training a statistical machine-learning model or a neural network to determine the confidence classifications. Relatedly, in one or more embodiments, training the genome-location-classification model to determine the confidence classifications comprises training a logistic regression model, a random forest classifier, or a convolutional neural network to determine the confidence classifications.

Further, in some circumstances, the confidence classifications indicate a degree to which nucleobases can be accurately determined at the particular genomic coordinates. Relatedly, in some cases, determining the confidence classifications comprises determining a confidence classification for a single nucleotide variant, a nucleobase insertion, a nucleobase deletion, a part of a structural variation, or a part of a copy number variation at a genomic coordinate.

As further suggested above, in one or more embodiments, training the genome-location-classification model to determine the confidence classifications comprises: comparing, for the genomic coordinate, a projected confidence classification to a ground-truth classification reflecting a Mendelian-inheritance pattern or a replicate concordance of nucleobase calls at the genomic coordinate; determining a loss from the comparison of the projected confidence classification to the ground-truth classification; and adjusting a parameter of the genome-location-classification model based on the determined loss.

As further shown in FIG. 11A, the acts 1100a include an act 1106 of determining a set of confidence classifications for a set of genomic coordinates. In particular, in certain implementations, the act 1106 includes determining, utilizing the genome-location-classification model, a set of confidence classifications for a set of genomic coordinates based on a set of sequencing metrics for one or more sample nucleic-acid sequences. In some cases, the act 1106 includes determining, utilizing the genome-location-classification model, a confidence classification for the genomic coordinate based on the contextual nucleic-acid subsequence.

For example, in one or more implementations, determining a confidence classification from the set of confidence classifications comprises determining the confidence classification for a genomic coordinate comprising a genetic modification or an epigenetic modification. Relatedly, in some embodiments, determining a confidence classification from the set of confidence classifications comprises determining the confidence classification for a single nucleotide variant, a nucleobase insertion, a nucleobase deletion, or a part of a structural variation at a genomic coordinate.

Further, in some circumstances, determining a confidence classification from the set of confidence classifications comprises determining at least one of a high-confidence classification, an intermediate-confidence classification, or a low-confidence classification for a genomic coordinate. Additionally or alternatively, determining a confidence classification from the set of confidence classifications comprises determining a confidence score within a range of confidence scores indicating a degree to which nucleobases can be accurately determined at a genomic coordinate.

As further shown in FIG. 11A, the acts 1100a include an act 1108 of generating at least one digital file comprising the set of confidence classifications. In particular, in certain implementations, the act 1108 includes generating at least one digital file comprising the set of confidence classifications for the set of genomic coordinates. Similarly, in some embodiments, the act 1108 includes generating a digital file comprising the confidence classification for the genomic coordinate of the variant-nucleobase call.

In addition to the acts 1102-1108, in certain implementations, the acts 1100a include determining, from the example nucleic-acid sequence, a contextual nucleic-acid subsequence surrounding a variant-nucleobase call; and training the genome-location-classification model to determine a confidence classification for a genomic coordinate of the variant-nucleobase call based on: the contextual nucleic-acid subsequence; a subset of sequencing metrics for a subset of genomic coordinates corresponding to the contextual nucleic-acid subsequence; and a subset of ground-truth classifications for the subset of genomic coordinates corresponding to the contextual nucleic-acid subsequence.

Turning now to FIG. 11B, this figure illustrates a flowchart of a series of acts 1100b of training a machine-learning model to determine variant confidence classifications for genomic coordinates in accordance with one or more embodiments. While FIG. 11B illustrates acts according to one embodiment, alternative embodiments may omit, add to, reorder, and/or modify any of the acts shown in FIG. 11B. The acts of FIG. 11B can be performed as part of a method. Alternatively, a non-transitory computer readable storage medium can comprise instructions that, when executed by one or more processors, cause a computing device to perform the acts depicted in FIG. 11B. In still further embodiments, a system comprising at least one processor and a non-transitory computer readable medium comprising instructions that, when executed by one or more processors, cause the system to perform the acts of FIG. 11B.

As shown in FIG. 11B, the acts 1100b include an act 1110 of determining sequencing metrics for sample nucleic-acid sequences from an admixture of genome samples. In particular, in some embodiments, the act 1110 includes determining sequencing metrics for comparing sample nucleic-acid sequences from genome samples to genomic coordinates of an example nucleic-acid sequence. For instance, in some cases, determining the sequencing metrics comprises determining mapping-quality metrics, forward-reverse-depth metrics, and nucleobase-call-quality metrics for the sample nucleic-acid sequences. In one or more embodiments, the sample nucleic-acid sequences are determined using a single sequencing pipeline comprising a nucleic-acid-sequence-extraction method, a sequencing device, and a sequence-analysis software.

As further shown in FIG. 11B, the acts 1100b include an act 1112 of generating, for variant-nucleobase calls, ground-truth classifications for genomic coordinates based on one or more of the sequencing metrics. For instance, the act 1112 can include generating, for particular variant-nucleobase calls, ground-truth classifications for particular genomic coordinates based on one or more of the sequencing metrics or variant-call data for an admixture of genome samples. As a further example, the act 1112 can include generating the ground-truth classifications based on the one or more of the sequencing metrics comprising mapping-quality metrics, forward-reverse-depth metrics, and nucleobase-call-quality metrics for the sample nucleic-acid sequences.

As suggested above, in certain embodiments, generating, for the particular variant-nucleobase calls, the ground-truth classifications for the particular genomic coordinates based on the variant-call data for the admixture of genome samples comprises determining one or more of a rate of precision or a rate of recall for determining a set of variant-nucleobase calls for one or more sample nucleic-acid sequences from the admixture of genome samples at the particular genomic coordinates; and generating the ground-truth classifications based on one or more of the rate of precision or the rate of recall for determining the set of variant-nucleobase calls. Further, in some implementations, generating, for the particular variant-nucleobase calls, the ground-truth classifications for the particular genomic coordinates based on the variant-call data for the admixture of genome samples comprises determining variant-allele frequencies of a set of variant-nucleobase calls for one or more sample nucleic-acid sequences from the admixture of genome samples; determining one or more of a rate of precision or a rate of recall for determining different variant-nucleobase calls for one or more sample nucleic-acid sequences from the admixture of genome samples at the particular genomic coordinates and at different variant-allele frequencies from the variant-allele frequencies; and generating the ground-truth classifications based on one or more of the rate of precision or the rate of recall for determining different variant-nucleobase calls at the different variant-allele frequencies.

Relatedly, in some cases, generating, for the particular variant-nucleobase calls, the ground-truth classifications for the particular genomic coordinates based on the variant-call data for the admixture of genome samples comprises determining somatic-quality metrics for nucleobase calls from one or more sample nucleic-acid sequences from the admixture of genome samples; generating somatic-quality-metric thresholds for differentiating different ground-truth classifications for the particular genomic coordinates; and generating tiered ground-truth classifications for the particular genomic coordinates according to the somatic-quality-metric thresholds. In some such cases, generating the tiered ground-truth classifications comprises generating only a subset of tiered ground-truth classifications according to the somatic-quality-metric thresholds.

Further, in some embodiments, generating, for the particular variant-nucleobase calls, the ground-truth classifications for the particular genomic coordinates based on the variant-call data for the admixture of genome samples comprises determining variant-allele frequencies of a set of variant-nucleobase calls for one or more sample nucleic-acid sequences from the admixture of genome samples; determining a rate of precision and a rate of recall for determining the set of variant-nucleobase calls for the one or more sample nucleic-acid sequences from the admixture of genome samples at the particular genomic coordinates and at different variant-allele frequencies from the variant-allele frequencies; determining F-scores for determining the different variant-nucleobase calls at the particular genomic coordinates based on the rate of precision and the rate of recall; and generating the ground-truth classifications based further on the F-scores for determining the different variant-nucleobase calls.

In addition to the acts 1110 and 1112, in some embodiments, the acts 1100b further include determining, from one or more example nucleic-acid sequences, contextual nucleic-acid subsequences surrounding variant-nucleobase calls in one or more sample nucleic-acid sequences at one or more genomic coordinates. In certain implementations, the one or more example nucleic-acid sequences comprise a reference genome or nucleic-acid sequences of ancestral haplotype.

As further shown in FIG. 11B, the acts 1100b include an act 1114 of training a genome-location-classification model to determine variant confidence classification for genomic coordinates based on the ground-truth classifications. In particular, in some embodiments, the act 1114 includes training a genome-location-classification model to determine, for variant-nucleobase calls, variant confidence classifications for the genomic coordinates based on the sequencing metrics and the ground-truth classifications. Further, in some cases, the act 1114 includes training a genome-location-classification model to determine, for the variant-nucleobase calls, variant confidence classifications for the genomic coordinates based on the contextual nucleic-acid subsequences and the ground-truth classifications.

As suggested above, in certain embodiments, the variant confidence classifications indicate a degree to which somatic-nucleobase variants reflecting cancer or somatic mosaicism can be accurately determined at the genomic coordinates. By contrast, in some cases, the variant confidence classifications indicate a degree to which germline-nucleobase variants reflecting germline mosaicism can be accurately determined at the genomic coordinates.

As further shown in FIG. 11B, the acts 1100b include an act 1116 of determining a set of variant confidence classifications for a set of genomic coordinates. In particular, in certain implementations, the act 1116 includes determining, utilizing the genome-location-classification model, a set of variant confidence classifications for a set of genomic coordinates based on a set of sequencing metrics for one or more sample nucleic-acid sequences. In some cases, the act 1116 includes determining, utilizing the genome-location-classification model, a set of variant confidence classifications for a set of genomic coordinates based on a set of contextual nucleic-acid subsequences surrounding a corresponding set of variant-nucleobase calls. For instance, determining the set of sequencing metrics can include determining the set of sequencing metrics for the one or more sample nucleic-acid sequences from one or more genome samples.

As further examples, in some cases, the act 1116 includes determining a variant confidence classification from the set of variant confidence classifications by determining the variant confidence classification for a genomic coordinate based on a contextual nucleic-acid subsequence surrounding a somatic-nucleobase variant that reflects cancer or somatic mosaicism. By contrast, in certain cases, the act 1116 includes determining a variant confidence classification from the set of variant confidence classifications by determining the variant confidence classification for a genomic coordinate based on a contextual nucleic-acid subsequence surrounding a germline-nucleobase variant that reflects germline mosaicism. Further, in one or more embodiments, the act 1116 includes determining a variant confidence classification from the set of variant confidence classifications by determining a variant confidence score within a range of variant confidence scores indicating a degree to which nucleobase variants can be accurately determined at a genomic coordinate.

In addition to the acts 1110-1116, in certain implementations, the acts 1100b include determining the admixture of genome samples by determining a combination of a first subset of nucleic-acid sequences from a first genome sample and a second subset of nucleic-acid sequences from a second genome sample that together simulate variant-allele frequencies of a genome sample with cancer or mosaicism. Similarly, in some cases, the acts 1100b include determining the admixture of genome samples by determining a combination of a first percentage of nucleic-acid sequences from a first naturally occurring genome sample and a second percentage of nucleic-acid sequences from a second naturally occurring genome sample that together simulate variant-allele frequencies of a genome sample with cancer or mosaicism.

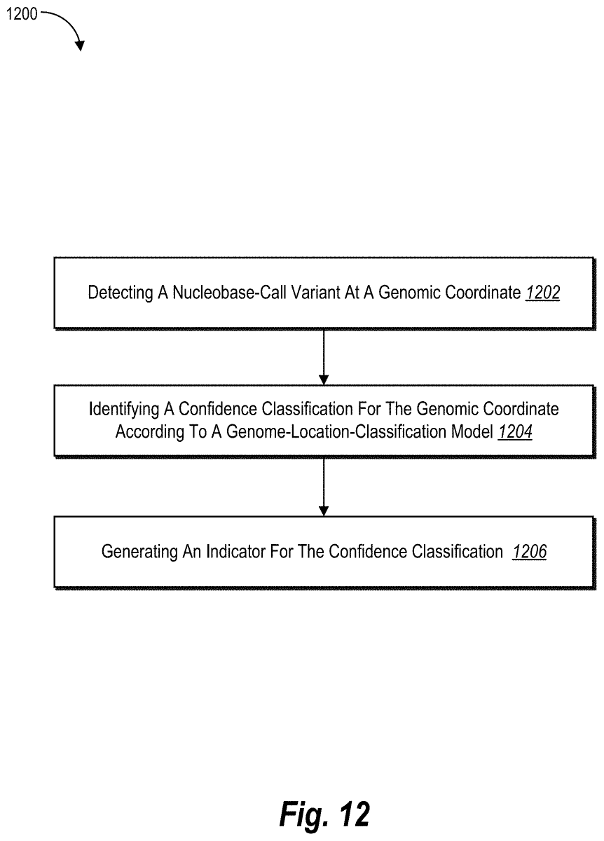

Turning now to FIG. 12, this figure illustrates a flowchart of a series of acts 1200 for generating an indicator of a confidence classification for a genomic coordinate of a variant-nucleobase call from a digital file in accordance with one or more embodiments. While FIG. 12 illustrates acts according to one embodiment, alternative embodiments may omit, add to, reorder, and/or modify any of the acts shown in FIG. 12. The acts of FIG. 12 can be performed as part of a method. Alternatively, a non-transitory computer readable storage medium can comprise instructions that, when executed by one or more processors, cause a computing device to perform the acts depicted in FIG. 12. In still further embodiments, a system comprising at least one processor and a non-transitory computer readable medium comprising instructions that, when executed by one or more processors, can cause the system to perform the acts of FIG. 12.

As shown in FIG. 12, the acts 1200 include an act 1202 of detecting a variant-nucleobase call at a genomic coordinate. In particular, in some embodiments, the act 1202 includes detecting a variant-nucleobase call at a genomic coordinate within a sample nucleic-acid sequence. As indicated above, in some cases, detecting the variant-nucleobase call at the genomic coordinate comprises detecting a single nucleotide variant, a nucleobase insertion, a nucleobase deletion, or a part of a structural variation.

As further shown in FIG. 12, the acts 1200 include an act 1204 of identifying a confidence classification for the genomic coordinate according to a genome-location-classification model. In particular, in some embodiments, the act 1204 includes identifying, from a digital file, a confidence classification for the genomic coordinate according to a genome-location-classification model.

As suggested above, in certain embodiments, identifying the confidence classification for the genomic coordinate comprises identifying, from the digital file, the confidence classification indicating a degree to which nucleobases can be accurately determined at the genomic coordinate. Further, in some implementations, identifying, from the digital file, the confidence classification comprises identifying the confidence classification from an annotation or a score for the genomic coordinate within the digital file. Relatedly, in one or more embodiments, identifying, from the digital file, the confidence classification comprises identifying at least one of a high-confidence classification, an intermediate-confidence classification, or a low-confidence classification for the genomic coordinate.

As further shown in FIG. 12, the acts 1200 include an act 1206 of generating an indicator for the confidence classification. In particular, in certain implementations, the act 1206 includes generating, for display within a graphical user interface, an indicator of the confidence classification for the genomic coordinate of the variant-nucleobase call.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeg™ platform (Illumina, Inc., San Diego, Calif.) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the genome-classification system 106 can include software, hardware, or both. For example, the components of the genome-classification system 106 can include one or more instructions stored on a computer-readable storage medium and executable by processors of one or more computing devices (e.g., the user client device 108). When executed by the one or more processors, the computer-executable instructions of the genome-classification system 106 can cause the computing devices to perform the bubble detection methods described herein. Alternatively, the components of the genome-classification system 106 can comprise hardware, such as special purpose processing devices to perform a certain function or group of functions. Additionally, or alternatively, the components of the genome-classification system 106 can include a combination of computer-executable instructions and hardware.

Furthermore, the components of the genome-classification system 106 performing the functions described herein with respect to the genome-classification system 106 may, for example, be implemented as part of a stand-alone application, as a module of an application, as a plug-in for applications, as a library function or functions that may be called by other applications, and/or as a cloud-computing model. Thus, components of the genome-classification system 106 may be implemented as part of a stand-alone application on a personal computing device or a mobile device. Additionally, or alternatively, the components of the genome-classification system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 13 illustrates a block diagram of a computing device 1300 that may be configured to perform one or more of the processes described above. One will appreciate that one or more computing devices such as the computing device 1300 may implement the genome-classification system 106 and the sequencing system 104. As shown by FIG. 13, the computing device 1300 can comprise a processor 1302, a memory 1304, a storage device 1306, an I/O interface 1308, and a communication interface 1310, which may be communicatively coupled by way of a communication infrastructure 1312. In certain embodiments, the computing device 1300 can include fewer or more components than those shown in FIG. 13. The following paragraphs describe components of the computing device 1300 shown in FIG. 13 in additional detail.

In one or more embodiments, the processor 1302 includes hardware for executing instructions, such as those making up a computer program. As an example, and not by way of limitation, to execute instructions for dynamically modifying workflows, the processor 1302 may retrieve (or fetch) the instructions from an internal register, an internal cache, the memory 1304, or the storage device 1306 and decode and execute them. The memory 1304 may be a volatile or non-volatile memory used for storing data, metadata, and programs for execution by the processor(s). The storage device 1306 includes storage, such as a hard disk, flash disk drive, or other digital storage device, for storing data or instructions for performing the methods described herein.

The I/O interface 1308 allows a user to provide input to, receive output from, and otherwise transfer data to and receive data from computing device 1300. The I/O interface 1308 may include a mouse, a keypad or a keyboard, a touch screen, a camera, an optical scanner, network interface, modem, other known I/O devices or a combination of such I/O interfaces. The I/O interface 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1308 is configured to provide graphical data to a display for presentation to a user. The graphical data may be representative of one or more graphical user interfaces and/or any other graphical content as may serve a particular implementation.

The communication interface 1310 can include hardware, software, or both. In any event, the communication interface 1310 can provide one or more interfaces for communication (such as, for example, packet-based communication) between the computing device 1300 and one or more other computing devices or networks. As an example, and not by way of limitation, the communication interface 1310 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1310 may facilitate communications with various types of wired or wireless networks. The communication interface 1310 may also facilitate communications using various communication protocols. The communication infrastructure 1312 may also include hardware, software, or both that couples components of the computing device 1300 to each other. For example, the communication interface 1310 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium comprising instructions that, when executed by the at least one processor, cause the system to:
determine sequencing metrics for comparing sample nucleic-acid sequences with genomic coordinates of an example nucleic-acid sequence;
train a genome-location-classification model to determine confidence classifications for the genomic coordinates based on the sequencing metrics and ground-truth classifications for particular genomic coordinates;
determine, utilizing the genome-location-classification model, a set of confidence classifications for a set of genomic coordinates based on a set of sequencing metrics for one or more sample nucleic-acid sequences; and
generate at least one digital file comprising the set of confidence classifications for the set of genomic coordinates.

2. The system of claim 1, wherein the confidence classifications indicate a degree to which nucleobases can be accurately determined at the particular genomic coordinates.

3. The system of claim 1, wherein the sample nucleic-acid sequences are determined using a single sequencing pipeline comprising a nucleic-acid-sequence-extraction method, a sequencing device, and a sequence-analysis software.

4. The system of claim 1, further comprising instructions that, when executed by the at least one processor, cause the system to determine a confidence classification from the set of confidence classifications by determining the confidence classification for a genomic coordinate comprising a genetic modification or an epigenetic modification.

5. The system of claim 1, further comprising instructions that, when executed by the at least one processor, cause the system to determine the sequencing metrics by determining one or more of:
alignment metrics for quantifying alignment of the sample nucleic-acid sequences with the genomic coordinates of the example nucleic-acid sequence;
depth metrics for quantifying depth of nucleobase calls for the sample nucleic-acid sequences at the genomic coordinates of the example nucleic-acid sequence; or
call-data-quality metrics for quantifying quality of the nucleobase calls for the sample nucleic-acid sequences at the genomic coordinates of the example nucleic-acid sequence.

6. The system of claim 5, further comprising instructions that, when executed by the at least one processor, cause the system to:
determine the alignment metrics by determining one or more of deletion-entropy metrics, deletion-size metrics, mapping-quality metrics, positive-insert-size metrics, negative-insert-size metrics, soft-clipping metrics, read-position metrics, or read-reference-mismatch metrics for the sample nucleic-acid sequences;
determine the depth metrics by determining one or more of forward-reverse-depth metrics, normalized-depth metrics, depth-under metrics, depth-over metrics, or peak-count metrics; or
determine the call-data-quality metrics by determining one or more of nucleobase-call-quality metrics, callability metrics, or somatic-quality metrics for the sample nucleic-acid sequences.

7. The system of claim 1, further comprising instructions that, when executed by the at least one processor, cause the system to determine a confidence classification from the set of confidence classifications by determining at least one of a high-confidence classification, an intermediate-confidence classification, or a low-confidence classification for a genomic coordinate.

8. The system of claim 1, further comprising instructions that, when executed by the at least one processor, cause the system to determine a confidence classification from the set of confidence classifications by determining a confidence score within a range of confidence scores indicating a degree to which nucleobases can be accurately determined at a genomic coordinate.

9. The system of claim 1, further comprising instructions that, when executed by the at least one processor, cause the system to train the genome-location-classification model to determine the confidence classifications by training a statistical machine-learning model or a neural network to determine the confidence classifications.

10. The system of claim 1, further comprising instructions that, when executed by the at least one processor, cause the system to:
determine, from the example nucleic-acid sequence, a contextual nucleic-acid subsequence surrounding a variant-nucleobase call; and
train the genome-location-classification model to determine a confidence classification for a genomic coordinate of the variant-nucleobase call based on:
the contextual nucleic-acid subsequence;
a subset of sequencing metrics for a subset of genomic coordinates corresponding to the contextual nucleic-acid subsequence; and
a subset of ground-truth classifications for the subset of genomic coordinates corresponding to the contextual nucleic-acid subsequence.

11. A non-transitory computer-readable medium storing instructions that, when executed by at least one processor, cause a computing device to:
detect a variant-nucleobase call at a genomic coordinate within a sample nucleic-acid sequence;
identify, from a digital file, a confidence classification for the genomic coordinate according to a genome-location-classification model; and
generate, for display within a graphical user interface, an indicator of the confidence classification for the genomic coordinate of the variant-nucleobase call.

12. The non-transitory computer-readable medium of claim 11, further storing instructions that, when executed by the at least one processor, cause the computing device to identify, from the digital file, the confidence classification for the genomic coordinate by identifying the confidence classification indicating a degree to which nucleobases can be accurately determined at the genomic coordinate.

13. The non-transitory computer-readable medium of claim 11, further storing instructions that, when executed by the at least one processor, cause the computing device to identify, from the digital file, the confidence classification by identifying the confidence classification from an annotation or a score for the genomic coordinate within the digital file.

14. The non-transitory computer-readable medium of claim 11, further storing instructions that, when executed by the at least one processor, cause the computing device to identify, from the digital file, the confidence classification by identifying at least one of a high-confidence classification, an intermediate-confidence classification, or a low-confidence classification for the genomic coordinate.

15. A method comprising:
determining, from an example nucleic-acid sequence, a contextual nucleic-acid subsequence surrounding a variant-nucleobase call in a sample nucleic-acid sequence at a genomic coordinate from genomic coordinates of an example nucleic-acid sequence;
training a genome-location-classification model to determine confidence classifications for the genomic coordinate based on the contextual nucleic-acid subsequence and a ground-truth classification for the genomic coordinate;
determining, utilizing the genome-location-classification model, a confidence classification for the genomic coordinate based on the contextual nucleic-acid subsequence; and
generating at least one digital file comprising the confidence classification for the genomic coordinate of the variant-nucleobase call.

16. The method of claim 15, wherein determining the confidence classification comprises determining the confidence classification for a single nucleotide variant, a nucleobase insertion, a nucleobase deletion, a part of a structural variation, or a part of a copy number variation at a genomic coordinate.

17. The method of claim 15, wherein determining the confidence classification comprises determining a confidence score within a range of confidence scores indicating a degree to which nucleobases can be accurately determined at a genomic coordinate.

18. The method of claim 15, wherein training the genome-location-classification model to determine the confidence classifications comprises training a logistic regression model, a random forest classifier, or a convolutional neural network to determine the confidence classifications.

19. The method of claim 15, wherein training the genome-location-classification model to determine the confidence classifications comprises:
comparing, for the genomic coordinate, a projected confidence classification to a ground-truth classification reflecting a Mendelian-inheritance pattern or a replicate concordance of nucleobase calls at the genomic coordinate;
determining a loss from the comparison of the projected confidence classification to the ground-truth classification; and
adjusting a parameter of the genome-location-classification model based on the determined loss.

20. The method of claim 15, wherein the example nucleic-acid sequence comprises a reference genome or a nucleic-acid sequence of an ancestral haplotype.
